(19) United States
(12) Patent Application Publication
(10) Pub. No.: US 2026/0083769 A1
(43) Pub. Date: Mar. 26, 2026

(54) 2-Aminopropane-1,3-Diol-Capped Cationic Peptoids for Nucleic Acid Delivery

(71) Applicant: NUTCRACKER THERAPEUTICS, INC., Emeryville, CA (US)

(72) Inventors: Colin James McKinlay, Emeryville, CA (US); Nicole PECK, Emeryville, CA (US); Timothy BLAKE, Emeryville, CA (US); Juan ECHEVERRI, Emeryville, CA (US)

(21) Appl. No.: 19/108,894

(22) PCT Filed: Sep. 8, 2023
(86) PCT No.: PCT/US2023/032273

§ 371 Date: Mar. 5, 2025

Related U.S. Application Data
(60) Provisional application No. 63/405,043, filed on Sep. 9, 2022.

(51) Int. Cl. A61K 31/7105; A61K 39/00; A61K 47/18; A61K 47/42; C07K 5/103; C07K 7/06

(52) U.S. Cl. CPC A61K 31/7105; A61K 39/00; A61K 47/18; A61K 47/42; C07K 5/1008; C07K 7/06; A61K 2039/53

(57) ABSTRACT

The present disclosure provides delivery vehicle compositions comprising hydroxyalkyl-capped cationic peptoids, such as 2-aminopropane-1,3-diol-capped cationic peptoids, and complexes of the delivery vehicles with polyanionic compounds, such as nucleic acids. The disclosure further provides methods of making and using the delivery vehicle compositions and complexes, such as for the delivery polyanionic compounds (e.g., nucleic acids) to cells. The disclosure also provides methods of eliciting an immune response with the delivery vehicle complexes of the disclosure.

### DESCRIPTION OF RELATED TECHNOLOGY

Therapeutic nucleic acids, such as mRNA, small interfering RNA (siRNA), small activating RNA (saRNA), micro RNA (miRNA), antisense oligonucleotides, ribozymes, plasmids, and immune stimulating nucleic acids, have great promise for the prevention and treatment of diseases at the genetic level. Nucleic acids, however, typically undergo fast degradation in blood, renal clearance, poor cellular uptake, and inefficient endosomal escape. Therefore, a safe and effective system for delivering nucleic acids to a cell nucleus or cytosol is required for the nucleic acids to be therapeutically useful. Traditional methods for the cellular and in vivo delivery of polyanionic compounds, such as oligonucleotides, include viral vectors, cationic lipid nanoparticles (LNPs), and polycationic polymers. These delivery systems can be plagued by limitations such as poor stability, rapid clearance, poor toxicology, immune response concerns, and suboptimal expression of their polyanionic cargo.

### SUMMARY

There is a need for stable, safe, and efficacious systems for the delivery of nucleic acids to cells. Accordingly, the present disclosure relates to delivery vehicle compositions comprising 2-aminopropane-1,3-diol-capped cationic peptoids, and complexes of the delivery vehicle compositions with polyanionic compounds, such as nucleic acids. The disclosure further relates to methods of making and using the delivery vehicle compositions and complexes for the endocellular delivery of polyanionic compounds, such as mRNA, as well as methods of eliciting an immune response with the complexes of the disclosure.

Disclosed are N-substituted glycine oligomers (termed peptoids) and their use as the ionizable lipid component in delivery vehicle compositions. By parameterizing peptoid structural variations including modifications to both the charged and hydrophobic portions of the peptoid, Design of Experiments (DOE) methodology was used to survey a large swath of chemical space facilitating the discovery of trends between peptoid structural elements and particle properties. This optimization process resulted in a >10,000-fold increase in resulting mRNA expression in vivo from initial compounds to final lead candidates, which enables their use in therapeutic programs including enzyme replacement and in-vivo-produced antibody drugs. The disclosed peptoids lead to expression of therapeutically-relevant levels of proteins, e.g., an anti-RSV antibody in mice with minimal tolerability concerns or induced immune responses. The disclosed compounds and compositions thereof present a tunable delivery platform that can be optimized toward specific therapeutic programs.

In one aspect, the disclosure provides compounds having a structure of Formula (I):

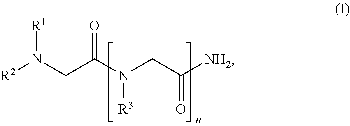

wherein n is 1, 2, 3, 4, 5, 6, 7, 8, 9, or 10; R¹ is H or C₂₋₅alkyl optionally substituted with 1-3 OH; R² is C₂₋₅alkylene-OH substituted with 1-3 additional OH; and each R³ independently is C₆₋₂₄alkyl or C₆₋₂₄alkenyl. In some cases, n is 3. In some cases, n is 4. In some cases, n is 6. In some cases, n is 8. In some cases, n is 9. In some cases, R¹ is H. In some cases, R² is C₂₋₅alkylene-OH substituted with 1 additional OH. In some cases, C₂₋₅alkylene is substituted with 2 or 3 additional OH. In some cases, R² is C₃₋₄alkylene substituted with 1-3 additional OH. In some cases, each R³ independently is C₆₋₁₈alkyl or C₆₋₁₈alkenyl. In some cases, each R³ independently is C₈₋₁₈alkyl or C₈₋₁₈alkenyl. In some cases, each R³ independently is selected from the group consisting of

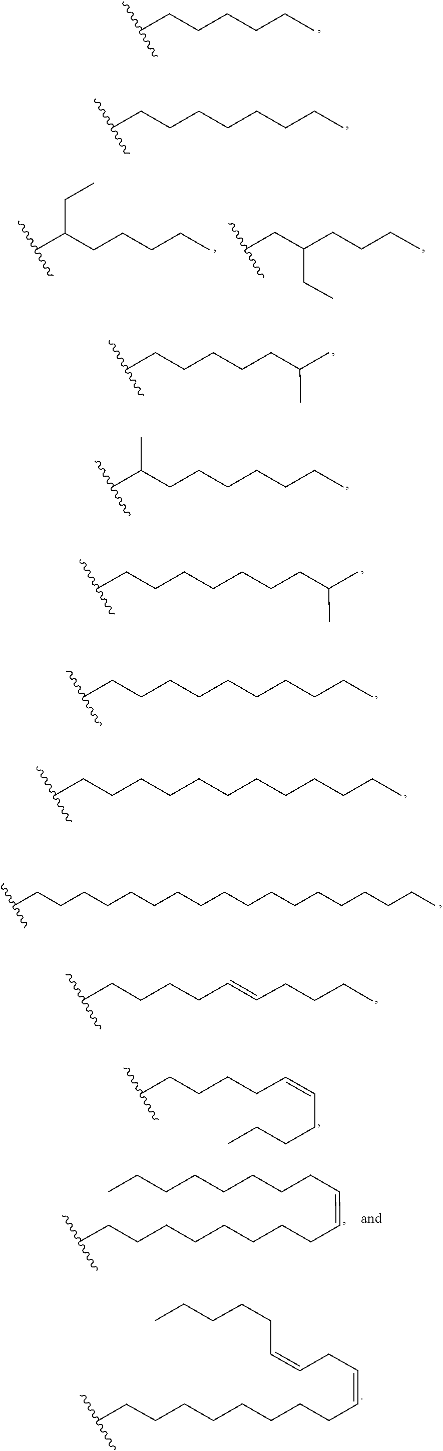

In some cases, each R³ independently is selected from the group consisting of

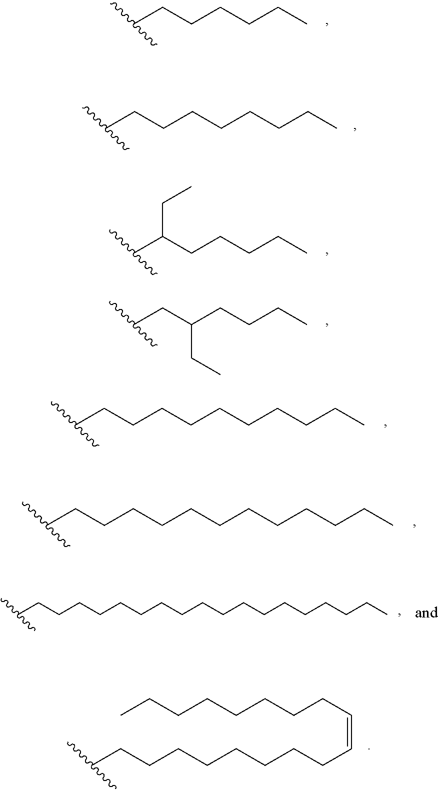

In some cases, at least one R³ is selected from the group consisting of

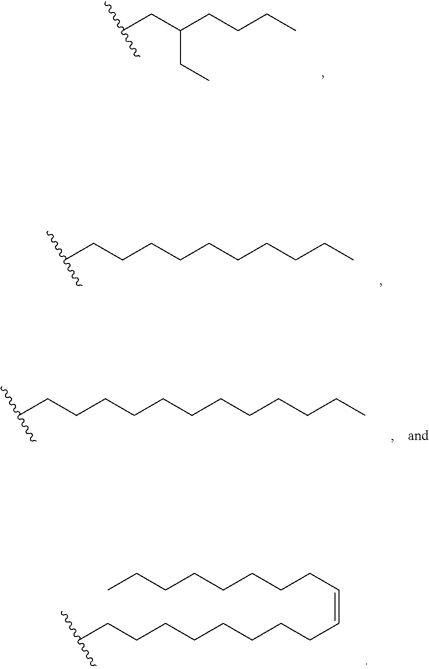

In some cases, at least one R³ is

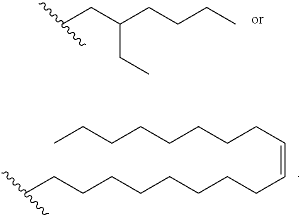

In some cases, the compound of Formula (I) has a structure selected from the group consisting of group consisting of:

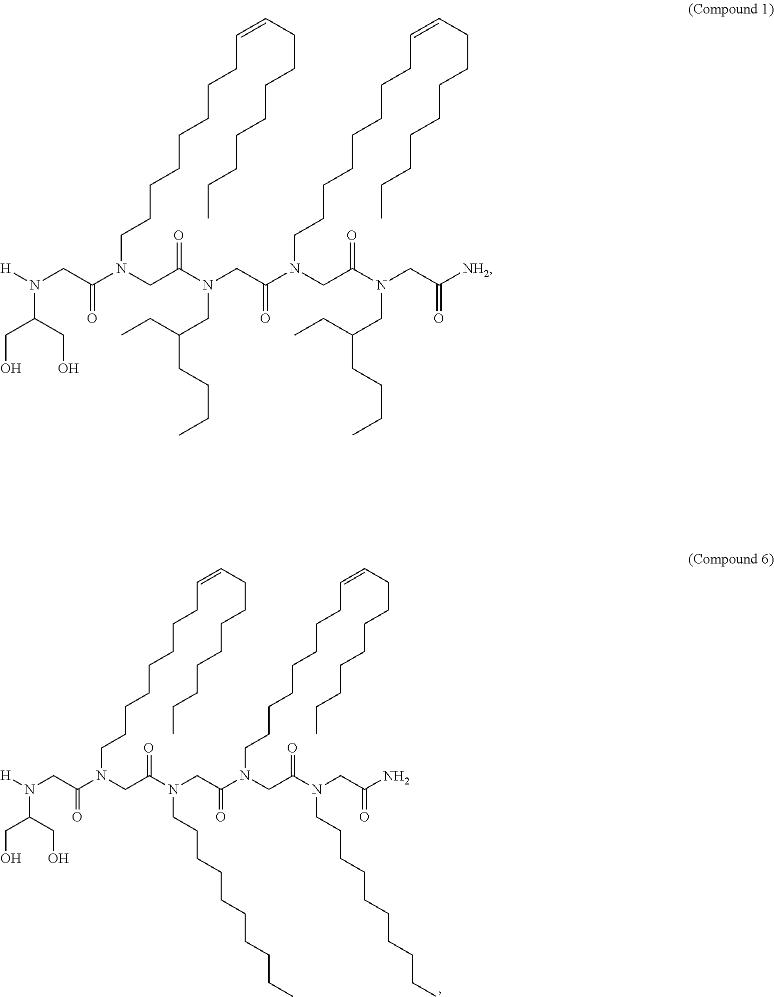 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNC(CO)CO ; CCCCCCCC/C=C\CCCCCCCCN(CC(=O)N(CCCCCCCCCC)CC(=O)N(CCCCCCCC/C=C\CCCCCCCC)CC(=O)N(CCCCCCCCCC)CC(N)=O)C(=O)CNC(CO)CO]

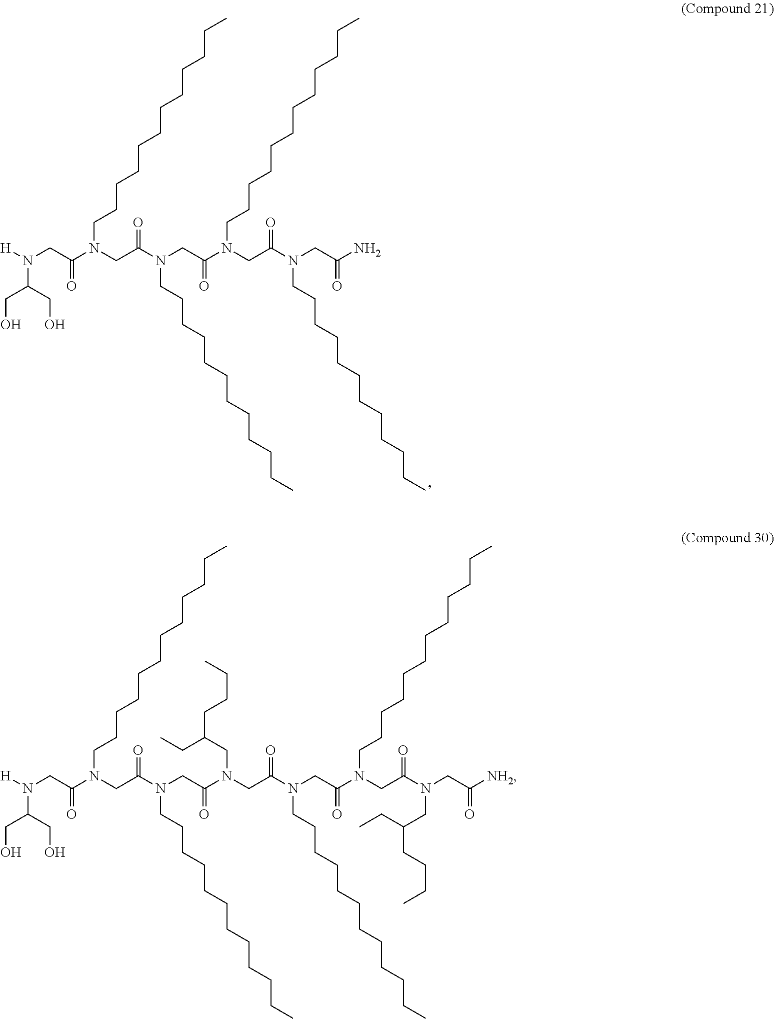 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNC(CO)CO ; CCCCCCCCCCCCN(CC(=O)N(CCCCCCCCCCCC)CC(=O)N(CC(=O)N(CCCCCCCCCCCC)CC(=O)N(CCCCCCCCCCCC)CC(=O)N(CC(N)=O)CC(CC)CCCC)CC(CC)CCCC)C(=O)CNC(CO)CO]

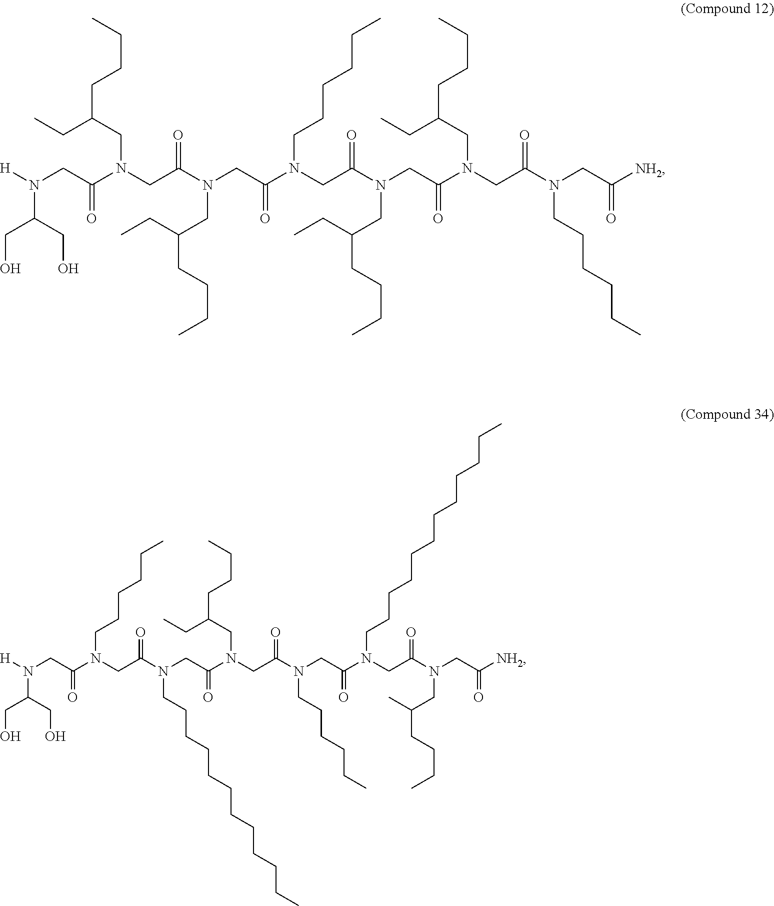 [SMILES: CCCCCCN(CC(N)=O)C(=O)CN(CC(CC)CCCC)C(=O)CN(CC(CC)CCCC)C(=O)CN(CCCCCC)C(=O)CN(CC(CC)CCCC)C(=O)CN(CC(CC)CCCC)C(=O)CNC(CO)CO ; CCCCCCCCCCCCN(CC(=O)N(CC(N)=O)CC(C)CCCC)C(=O)CN(CCCCCC)C(=O)CN(CC(CC)CCCC)C(=O)CN(CCCCCCCCCCCC)C(=O)CN(CCCCCC)C(=O)CNC(CO)CO]

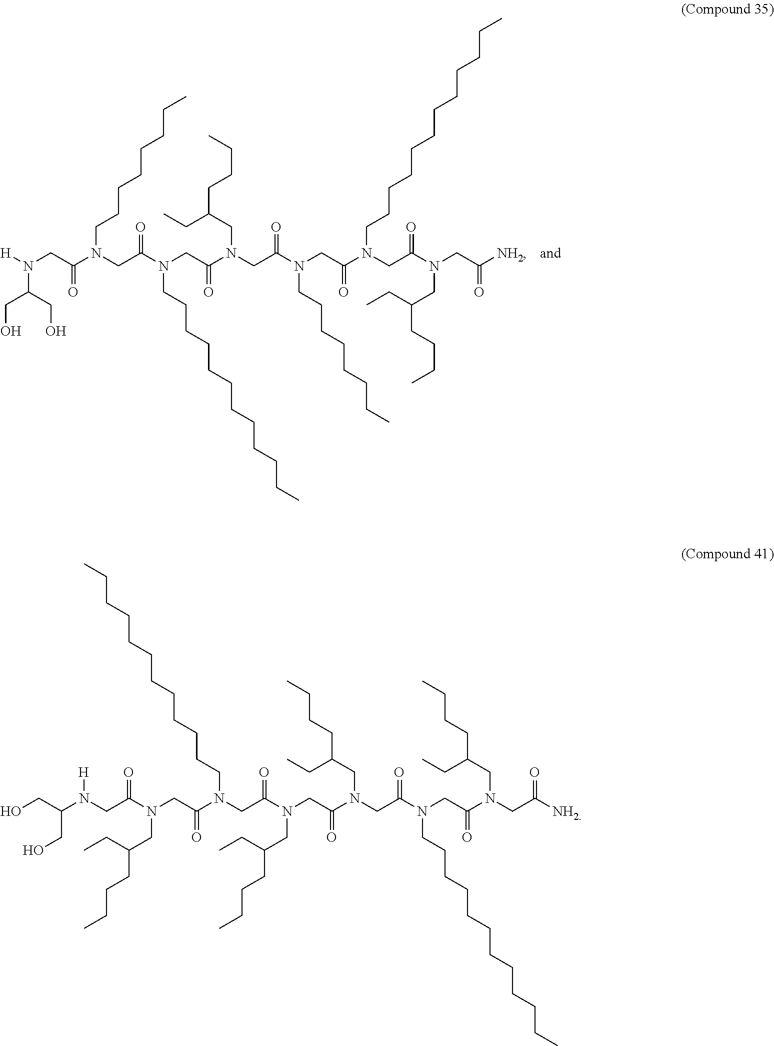 [SMILES: CCCCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCCCC)C(=O)CN(CCCCCCCC)C(=O)CNC(CO)CO ; CCCCCCCCCCCCN(CC(=O)N(CC(=O)N(CC(=O)N(CCCCCCCCCCCC)CC(=O)N(CC(N)=O)CC(CC)CCCC)CC(CC)CCCC)CC(CC)CCCC)C(=O)CN(CC(CC)CCCC)C(=O)CNC(CO)CO]

In some cases, the compound of Formula (I) is selected from Compound 1, Compound 6, Compound 21, and Compound 30. In some cases, the compound of Formula (I) is Compound 1. In some cases, the compound of Formula (I) is Compound 6. In some cases, the compound of Formula (I) is Compound 21. In some cases, the compound of Formula (I) is Compound 30. In some cases, the compound of Formula (I) is Compound 12. In some cases, the compound of Formula (I) is Compound 34. In some cases, the compound of Formula (I) is Compound 35. In some cases, the compound of Formula (I) is Compound 41. Further disclosed herein are pharmaceutically acceptable salts of the compounds of Formula (I).

Another aspect of the disclosure provides delivery vehicle compositions comprising the compounds disclosed herein or a pharmaceutically acceptable salt thereof. In some implementations, the composition further comprises one or more of a phospholipid, a sterol, and a PEGylated lipid. In some implementations, the compound or salt of Formula (I) is present in the delivery vehicle composition in an amount of about 30 mol % to about 60 mol %. In some implementations, the compound or salt of Formula (I) is present in the delivery vehicle composition in an amount of about 35 mol % to about 55 mol %. In various implementations, the compound or salt of Formula (I) is present in the delivery vehicle composition in an amount of about 30 mol % to about 45 mol %. In various implementations, the compound or salt of Formula (I) is present in the delivery vehicle composition in an amount of about 35 mol % to about 39 mol %. In some cases, the compound or salt of Formula (I) is present in the delivery vehicle composition in an amount of about 39 mol % to about 52 mol %. In various implementations, the compound or salt of Formula (I) is present in the delivery vehicle composition in an amount of about 30 mol % to about 35 mol %. In various implementations, the compound or salt of Formula (I) is present in the delivery vehicle composition in an amount of about 40 mol % to about 45 mol %. In various cases, the compound or salt of Formula (I) is present in an amount of about 42 mol % to about 49 mol %. In some implementations, the compound or salt of Formula (I) is present in an amount of about 50 mol % to about 52 mol %.

In various implementations, the composition comprises a phospholipid, a sterol, and a PEGylated lipid. In some cases, the composition consists essentially of a compound disclosed herein or a salt thereof, a phospholipid, a sterol, and a PEGylated lipid. In some cases, the composition comprises about 30 mol % to about 60 mol % of the compound of Formula (I); about 3 mol % to about 20 mol % of the phospholipid, about 25 mol % to about 60 mol % of the sterol, and about 1 mol % to about 5 mol % of the PEGylated lipid. In various cases, the composition comprises about 35 mol % to about 55 mol % of the compound or salt of Formula (I); about 5 mol % to about 15 mol % of the phospholipid, about 30 mol % to about 55 mol % of the sterol, and about 1 mol % to about 3 mol % of the PEGylated lipid. In some implementations, the composition comprises about 38 mol % to about 52 mol % of the compound or salt of Formula (I); about 9 mol % to about 12 mol % of the phospholipid, about 35 mol % to about 50 mol % of the sterol, and about 1 mol % to about 2 mol % of the PEGylated lipid. In various implementations, the composition comprises about 30 mol % to about 49 mol % of the compound of Formula (I); about 5 mol % to about 15 mol % of the phospholipid, about 30 mol % to about 55 mol % of the sterol, and about 1 mol % to about 3 mol % of the PEGylated lipid. In some cases, the composition comprises about 35 mol % to about 49 mol % of the compound or salt of Formula (I); about 7 mol % to about 12 mol % of the phospholipid, about 35 mol % to about 50 mol % of the sterol, and about 1 mol % to about 2 mol % of the PEGylated lipid. In some cases, the composition comprises about 30 mol % to about 45 mol % of the compound or salt of Formula (I); about 7 mol % to about 12 mol % of the phospholipid, about 40 mol % to about 55 mol % of the sterol, and about 1 mol % to about 3 mol % of the PEGylated lipid. In some cases, the composition comprises about 30 mol % to about 35 mol % of the compound or salt of Formula (I); about 7 mol % to about 12 mol % of the phospholipid, about 50 mol % to about 55 mol % of the sterol, and about 2 mol % to about 3 mol % of the PEGylated lipid. In some cases, the composition comprises about 40 mol % to about 45 mol % of the compound or salt of Formula (I); about 7 mol % to about 12 mol % of the phospholipid, about 40 mol % to about 45 mol % of the sterol, and about 1 mol % to about 2 mol % of the PEGylated lipid. In some cases, the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and combinations thereof. In some cases, the phospholipid is DOPE, DSPC, or a combination thereof. In various cases, the phospholipid is DSPC. In some implementations, the sterol is selected from the group consisting of cholesterol, fecosterol, sitosterol, ergosterol, campesterol, stigmasterol, brassicasterol, tomatidine, ursolic acid, alpha-tocopherol, and mixtures thereof. In some cases, the sterol is cholesterol. In some implementations, the PEGylated lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, a PEG-modified sterol, and a PEG-modified phospholipid. In various implementations, the PEG-modified lipid is selected from the group consisting of PEG-modified cholesterol, N-octanoyl-sphingosine-1-{succinyl[methoxy(polyethylene glycol)]}, N-palmitoyl-sphingosine-1-{succinyl[methoxy(polyethylene glycol)]}, PEG-modified DMPE (DMPE-PEG), PEG-modified DSPE (DSPE-PEG), PEG-modified DPPE (DPPE-PEG), PEG-modified DOPE (DOPE-PEG), dimyristoylglycerol-polyethylene glycol (DMG-PEG), distearoylglycerol-polyethylene glycol (DSG-PEG), dipalmitoylglycerol-polyethylene glycol (DPG-PEG), dioleoylglycerol-polyethylene glycol (DOG-PEG), and a combination thereof. In some cases, the PEG-modified lipid is dimyristoylglycerol-polyethylene glycol 2000 (DMG-PEG 2000). In various cases, the composition comprises about 38.2 mol % of Compound 1, 6, 21, or 30, about 11.8 mol % of DSPC, about 48.2 mol % of cholesterol, and about 1.9 mol % of DMG-PEG 2000. In some implementations, the composition comprises about 42.6 mol % of Compound 1, 6, 21, or 30, about 10.9 mol % of DSPC, about 44.7 mol % of cholesterol, and about 1.7 mol % of DMG-PEG 2000. In some implementations, the composition comprises about 48.2 mol % of Compound 1, 6, 21, or 30, about 9.9 mol % of DSPC, about 40.4 mol % of cholesterol, and about 1.6 mol % of DMG-PEG 2000. In various cases, the composition comprises about 51.3 mol % of Compound 1, 6, 21, or 30, about 9.3 mol % of DSPC, about 38 mol % of cholesterol, and about 1.5 mol % of DMG-PEG 2000. In various cases, the composition comprises about 44.4 mol % of Compound 1, 6, 21, or 30, about 10.6 mol % of DSPC, about 43.3 mol % of cholesterol, and about 1.7 mol % of DMG-PEG 2000. In various cases, the composition comprises about 44.4 mol % of Compound 1, 6, 21, or 30, about 10.6 mol % of DSPC, about 43.4 mol % of cholesterol, and about 1.7 mol % of DMG-PEG 2000. In various cases, the composition comprises about 33.1 mol % of Compound 1, 6, 21, or 30, about 10.6 mol % of DSPC, about 53.8 mol % of cholesterol, and about 2.5 mol % of DMG-PEG 2000.

Further disclosed herein are delivery vehicle complexes comprising a delivery vehicle composition described herein and a polyanionic compound. In some cases, the compound of Formula (I) or salt thereof is complexed to the polyanionic compound. In various cases, the compound or salt of Formula (I) and the polyanionic compound are present in a mass ratio of about 5:1 to about 25:1. In some implementations, the compound or salt of Formula (I) and the polyanionic compound are present in a mass ratio of about 7:1 to about 20:1. In various cases, the compound or salt of Formula (I) and the polyanionic compound are present in a mass ratio of about 10:1 to about 17:1. In some cases, the compound or salt of Formula (I) and the polyanionic compound are present in a mass ratio of about 19:1. In some cases, the compound or salt of Formula (I) and the polyanionic compound are present in a mass ratio of about 20:1. In some cases, the compound or salt of Formula (I) and the polyanionic compound are present in a mass ratio of about 10:1. In various cases, the compound or salt of Formula (I) and the polyanionic compound are present in a mass ratio of about 12:1. the compound or salt of Formula (I) and the polyanionic compound are present in a mass ratio of about 13:1. In some implementations, the compound or salt of Formula (I) and the polyanionic compound are present in a mass ratio of about 15:1. In various implementations, the compound or salt of Formula (I) and the polyanionic compound are present in a mass ratio of about 17:1. In some cases, the phospholipid and the polyanionic compound are present in a mass ratio of about 2:1 to about 10:1. In some cases, the phospholipid and the polyanionic compound are present in a mass ratio of about 2:1 to about 4:1. In various cases, the phospholipid and the polyanionic compound are present in a mass ratio of about 2:1 to about 3:1. In various cases, the phospholipid and the polyanionic compound are present in a mass ratio of about 4.0:1. In various cases, the phospholipid and the polyanionic compound are present in a mass ratio of about 2.7:1. In some implementations, the sterol and the polyanionic compound are present in a mass ratio of about 5:1 to about 8:1. In some implementations, the sterol and the polyanionic compound are present in a mass ratio of about 5:1 to about 6:1. In various implementations, the sterol and the polyanionic compound are present in a mass ratio of about 5.4:1. In some cases, the sterol and the polyanionic compound are present in a mass ratio of about 8.1:1. In some cases, the sterol and the polyanionic compound are present in a mass ratio of about 6.7:1. In some cases, the PEGylated lipid and the polyanionic compound are present in a mass ratio of about 0.5:1 to about 2.5:1. In various cases, the PEGylated lipid and the polyanionic compound are present in a mass ratio of about 1:1 to about 2:1. In some cases, the phospholipid and the polyanionic compound are present in a mass ratio of about 2.1:1. In some cases, the phospholipid and the polyanionic compound are present in a mass ratio of about 1.4:1. In various cases the delivery vehicle complex comprises Compound 1, 6, 21, or 30 having about a 10:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In various cases, the delivery vehicle complex comprises Compound 1, 6, 21, or 30 having about a 12:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In some cases, the delivery vehicle complex comprises Compound 1, 6, 21, or 30 having about a 15:1 mass ratio to the polyanionic compound, DSPC having about a 2.7:1 mass ratio to the polyanionic compound, and cholesterol having about a 5.4:1 mass ratio to the polyanionic compound, and DMG-PEG 2000 having about a 1.4:1 mass ratio to the polyanionic compound. In various cases, the delivery vehicle complex comprises Compound 1, 6, 21, or 30 having about a 17:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In various cases, the delivery vehicle complex comprises Compound 1, 6, 21, or 30 having about a 13:1 mass ratio to the polyanionic compound, DSPC having about a 2.7:1 mass ratio to the polyanionic compound, cholesterol having about a 5.4:1 mass ratio to the polyanionic compound, and DMG-PEG 2000 having about a 1.4:1 mass ratio to the polyanionic compound. In various cases, the delivery vehicle complex comprises Compound 1, 6, 21, or 30 having about a 19:1 mass ratio to the polyanionic compound, DSPC having about a 4.0:1 mass ratio to the polyanionic compound, cholesterol having about a 5.4:1 mass ratio to the polyanionic compound, and DMG-PEG 2000 having about a 2.1:1 mass ratio to the polyanionic compound. In various cases, the delivery vehicle complex comprises Compound 1, 6, 21, or 30 having about a 9.7:1 mass ratio to the polyanionic compound, DSPC having about a 2.7:1 mass ratio to the polyanionic compound, cholesterol having about a 6.7:1 mass ratio to the polyanionic compound, and DMG-PEG 2000 having about a 2.1:1 mass ratio to the polyanionic compound. In some cases, the compound of Formula (I) or salt thereof, phospholipid, sterol, and PEGylated lipid are present in a mass ratio of about 20:1.8:7.2:1.8.

In some cases, the complex exhibits a particle size of about 50 nm to about 200 nm and/or a polydispersity index (PDI) of less than 0.25. In various cases, the complex exhibits a particle size of about 60 nm to about 100 nm. In some implementations, the complex exhibits a particle size between about 60 nm to about 90 nm. In various implementations, the complex exhibits a particle size of about 105 nm to about 200 nm. In various cases, the complex exhibits a particle size of about 150 nm to about 200 nm. In some cases, the delivery vehicle complex exhibits a particle size of about 105 nm to about 200 nm. In some cases, the delivery vehicle complex exhibits a particle size of about 40 nm to about 115 nm, or about 55 nm to about 95 nm, or about 70 to about 80 nm, or about 75 nm. In various cases, the delivery vehicle complex exhibits a particle size of about 135 nm to about 225 nm, or about 155 nm to about 195 nm, or about 170 to about 180 nm, or about 175 nm. In various cases, at least 80% of the polyanionic compound is retained after storage at 4° C. for 48 days, or the delivery vehicle complex retains at least 80% of its original size after storage at 4° C. for 48 days, or both.

In some cases, the polyanionic compound comprises at least one nucleic acid. In various cases, the at least one nucleic acid comprises RNA, DNA, or a combination thereof. In various cases, the at least one nucleic acid comprises RNA. In some implementations, the RNA is mRNA encoding a peptide, a protein, or a functional fragment of the foregoing. In various implementations, the mRNA encodes for a viral peptide, a viral protein, or functional fragment of any of the foregoing. In some cases, the mRNA encodes for a human papillomavirus (HPV) protein or a functional fragment thereof. In various cases, the mRNA encodes for the HPV E6 protein and/or the HPV E7 protein, a variant thereof, or a functional fragment of any of the foregoing. In some cases, the HPV protein is from HPV subtype HPV 16, 18, 31, 33, 35, 39, 45, 51, 52, 56, 58, 59, 66, and/or 68. In various cases, the HPV protein is from HPV subtype HPV 16 and/or HPV 18. In some cases, the mRNA encodes for a viral spike protein or a functional fragment thereof. In various cases, the mRNA encodes for a SARS-CoV spike(S) protein, a variant thereof, or a functional fragment any of the foregoing. In some cases, the RNA encodes for a SARS-Related coronaviruses (e.g., severe acute respiratory syndrome coronavirus-2, (SARS-CoV-2), severe acute respiratory syndrome coronavirus (SARS-CoV), Middle East respiratory syndrome coronavirus (MERS-CoV), human coronavirus 229E (HCoV-229E), human coronavirus 0C43 (HCoV-0C43), human coronavirus HKU1 (HCoV-HKU1), or human coronavirus NL63 (HCoV-NL63). In some cases, the mRNA encodes for influenza hemagglutinin (HA), a variant thereof, or a functional fragment of any of the foregoing. In some implementations, the influenza A virus, has HA of a subtype selected from the group consisting of H1, H2, H3, H4, H5, H6, H7, H8, H9, H10, H11, H12, H13, H14, H15, and H16. In various implementations, the influenza subtype is HA strain H1, H2, H3 or H5. In various cases, one mRNA encodes for a SARS-CoV spike(S) protein and one mRNA that encodes for influenza hemagglutinin (HA), a variant thereof, or a functional fragment of any of the foregoing.

Further disclosed herein are pharmaceutical compositions comprising the delivery vehicle complexes of the disclosure and a pharmaceutically acceptable excipient. In some cases, the pharmaceutical composition is an intratumoral (IT) or intramuscular (IM) composition.

Also disclosed herein are methods of inducing an immune response in a subject in need thereof, comprising administering to the subject an effective amount of a delivery vehicle complex described herein or a pharmaceutical formulation comprising the delivery vehicle complex, thereby inducing an immune response in the subject. Further disclosed herein is a method of treating a viral infection in a subject in need thereof, comprising administering to the subject an effective amount of the delivery vehicle complex described herein or a pharmaceutical formulation comprising the delivery vehicle complex, thereby treating the viral infection in the subject. Also disclosed herein is a method of treating cancer in a subject in need thereof, comprising administering to the subject an effective amount of the delivery vehicle complex described herein or a pharmaceutical formulation comprising the delivery vehicle complex, thereby treating the cancer in the subject. In some cases, the cancer is cervical cancer, head and neck cancer, B-cell lymphoma, T-cell lymphoma, prostate cancer, lung cancer, or a combination thereof. In various cases, the administering is by intramuscular, intratumoral, intravenous, intraperitoneal, or subcutaneous delivery.

Also disclosed herein are methods of delivering a polyanionic compound to a cell comprising contacting the cell with a delivery vehicle complex described herein or a pharmaceutical formulation comprising the delivery vehicle complex. In some cases, the cell is a muscle cell, a tumor cell, or a combination thereof. In some cases, the polyanionic compound is an mRNA that encodes for a peptide, a protein, or a fragment of any of the foregoing, and the cell expresses the peptide, the protein, or the fragment after being contacted with the delivery vehicle complex.

Also disclosed herein are methods of forming a delivery vehicle complex disclosed herein, comprising contacting the compound or salt of Formula (I) with the polyanionic compound. In some cases, the method comprises admixing a solution comprising the compound or salt of Formula (I) with a solution comprising the polyanionic compound.

Also disclosed herein are vaccines comprising a delivery vehicle complex disclosed herein or a pharmaceutical composition disclosed herein. Also disclosed are vaccines comprising a delivery vehicle complex disclosed herein or a pharmaceutical composition disclosed herein for use in the treatment of cancer. Also disclosed are methods of treating or preventing cancer in a patient, comprising administering to the patient a delivery vehicle complex disclosed herein or a pharmaceutical composition disclosed herein. In various cases, the cancer is cervical cancer, head and neck cancer, B-cell lymphoma, T-cell lymphoma, prostate cancer, lung cancer, or a combination thereof.

It should be appreciated that all combinations of the foregoing concepts and implementations and additional concepts and implementations discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein.

Further aspects and advantages will be apparent to those of ordinary skill in the art from a review of the following detailed description, taken in conjunction with the drawings. While the compounds and methods disclosed herein are susceptible of implementations in various forms, the description hereafter includes specific implementations with the understanding that the disclosure is illustrative, and is not intended to limit the disclosure to the specific implementations described herein.

### BRIEF DESCRIPTION OF THE DRAWINGS

FIG. 1 shows the results of an in vivo assay of delivery vehicles comprising peptoids disclosed herein and control peptoids, evaluated using fLuc expression in BALB/c mice 6 hours after IV dose.

FIG. 2 shows the percent of total expression in spleen, lung, and liver for formulations of peptoids disclosed herein and control peptoids.

FIG. 3 shows the results of a firefly luciferase expression assay in BALB/c mice, evaluating the delivery efficacy of formulations of peptoids disclosed herein.

FIG. 4 shows contour plots evaluating the effect of carbon number and branching on delivery vehicle efficacy in a firefly luciferase expression assay.

FIG. 5 shows the results of an antiRSV titer for delivery vehicles comprising peptoids disclosed herein, dosed intravenously in BALB/c mice at 0.75 mg/kg mRNA and measured after 24 hours by IgG ELISA.

FIG. 6 shows the results of a dose escalation study for delivery vehicles comprising Compound 41 in a mouse model, measured by serum aRSV.

FIG. 7 shows that aRSV is detectable in serum beyond 5 days post-dosing.

FIG. 8 shows the results of a dose escalation study for delivery vehicles comprising Compound 41 in a mouse model, measured via a firefly luciferase expression assay.

FIG. 9 shows a cryo-TEM image of particles of Compound 41 in a D22 formulation (“41-D22”).

FIG. 10 shows that particles of 41-D22 remain stable at 4° C. or −80° C. storage for over 1 month with no growth or change in encapsulation observed.

FIG. 11 shows a comparison of firefly luciferase expression by delivery vehicles formulated with Compounds 31 and 41.

FIG. 12 shows a comparison of serum aRSV expression by delivery vehicles formulated with Compounds 31 and 41.

### DETAILED DESCRIPTION

The successful delivery of mRNA remains a significant challenge toward its adoption as a mainstream therapeutic modality. The approval of mRNA vaccines against SARS-CoV2 employing lipid nanoparticle (LNP) technology shows significant progress in this field, however the extension of mRNA drugs to applications beyond vaccines, such as protein replacement or immune-oncology therapies generating circulating antibodies, necessitates new delivery strategies and platforms. In these lipid nanoparticles, mRNA molecules are encapsulated in a combination of a cationic or ionizable lipid, helper lipids such as cholesterol and phospholipids, and a shielding lipid containing poly(ethylene glycol) (PEG). As mRNA is applied to a wider range of applications, including those which rely on IV delivery, the ability to tune the properties of ionizable lipids in particular has become a key focus of the field. Many reports have shown that tuning ionizable lipid properties impacts expression and tissue selectivity of delivery. Generation of novel ionizable lipids is often limited by efficient synthetic routes and requires significant synthetic effort and resources to expand lipid libraries to include new safe and effective candidates. Provided herein is a new class of peptoid based mRNA delivery vehicles, which utilize highly tunable peptoids as the ionizable lipid component to modulate delivery vehicle properties. A design of experiments approach was used to tune peptoid structure to optimize delivery vehicle function and demonstrate the optimization of delivery vehicles for systemic delivery of mRNA encoding e.g., aRSV.

Disclosed herein, in some examples, are delivery vehicle compositions comprising hydroxyalkyl-capped cationic peptoids, including, for example, 2-aminopropane-1,3-diol-capped cationic peptoids. The delivery vehicle compositions of the disclosure can form an electrostatic interaction between the 2-aminopropane-1,3-diol-capped cationic peptoids of the delivery vehicle composition and a polyanionic compound, such as a nucleic acid, to form a delivery vehicle complex, wherein the polyanionic compound functions as the cargo of the complex. The delivery vehicle complex is useful for the delivery of polyanionic compounds, such as nucleic acids (e.g., mRNA), into cells. Delivery vehicle complexes of the disclosure that include mRNA as the polyanionic cargo unexpectedly exhibit superior mRNA expression both in vitro and in vivo. When the mRNA of the delivery vehicle complex encodes, e.g., for a viral antigen, the delivery vehicle complexes can elicit humoral and cellular immune responses in vivo, thus functioning as a vaccine. The delivery vehicle complexes disclosed herein are further advantages in that they are stable, and demonstrate good tolerability and low toxicity.

As used herein, “peptoid” refers to a peptidomimetic compound in which one or more of the nitrogen atoms of the peptide backbone are substituted with side chains. As used herein, “polyanionic” refers to a compound having at least two negative charges, such as nucleic acids.

### Delivery Vehicle Compositions

Some example delivery vehicle compositions of the disclosure comprise one or more hydroxyalkyl-capped cationic peptoids, such as 2-aminopropane-1,3-diol-capped cationic peptoids. These positively charged peptoids can associate with a polyanionic compound, such as a nucleic acid, to form a delivery vehicle complex. In some implementations, the delivery vehicle compositions further comprise one or more of an anionic or zwitterionic component, such as a phospholipid; a neutral lipid, such as a sterol; and a shielding lipid, such as a PEGylated lipid. In various implementations, the delivery vehicle compositions further comprise an anionic or zwitterionic component (e.g., a phospholipid), a neutral lipid (e.g., a sterol), and a shielding lipid (e.g., a PEGylated lipid). In some cases, the delivery vehicle compositions consist essentially of hydroxyalkyl-capped cationic peptoids, such as 2-aminopropane-1,3-diol-capped cationic peptoids, an anionic or zwitterionic component (e.g., a phospholipid), a neutral lipid (e.g., a sterol), and a shielding lipid (e.g., a PEGylated lipid).

### Hydroxyalkyl-Capped Cationic Peptoid Component

Peptoids are a unique class of N-substituted alpha-amino acids structurally related to peptides, but which incorporate their diverse side chain functionality on the amide nitrogen rather than alpha carbon allowing for modular synthesis. In a non-limiting embodiment, peptoids can be synthesized by the submonomer approach wherein repeating cycles of acylation using bromoacetic acid and nucleophilic addition of primary amines can be accomplished on a solid support, producing peptoids with high yield and fidelity. Peptoids have been successfully used in many applications including anti-fouling and anti-bacterial agents, to drug delivery and anti-freeze additives in tissue storage, and even as complexing agents for nucleic acids, but they have never been explored as the ionizable component of a lipid nanoparticle for mRNA delivery.

Given their favorable properties and modular nature, it is thought that peptoids make an excellent platform for the discovery of new ionizable lipids and exploration of structure-activity relationships. Peptoids are easily synthesized using a solid-phase synthesizer and have an even larger monomer pool than traditional peptides. Traditionally, discovery of new lipids for nucleic acid delivery has been highly empirical, therefore successful systems are often those which can introduce high degrees of structural diversity and rapidly explore a wide range of chemical space. Utilizing the solid-phase submonomer method, the peptoid based platform disclosed herein takes advantage of the tunable and programmable nature of peptoid structures to cover a wide range of chemical space more easily than can be achieved with traditional lipid synthesis. When formulated with mRNA, DSPC, Cholesterol and DMG-PEG, delivery vehicles are generated and characterized for physical properties and biological activity. Through iterative synthesis and particle screening, the relationship between peptoid structure and delivery vehicle activity can be explored.

The first phase of screening focused on optimization of the cationic group on the peptoid molecules. The identity of the basic group used in an ionizable lipid significantly drives the particle pKa which is correlated with efficacy and specificity of nanoparticle expression. Particle pKas of 6-6.5 generally showing the highest efficacy in selective liver expression. However, it is yet unknown how a peptoid's molecular pKa correlate to the observed pKa once formulated in a delivery vehicle disclosed herein, therefore a variety of headgroup amines with varying basicity were selected.

Without being bound by any particular theory, it is thought that modifications to the lipophilic portion of the peptoid while keeping the leading 1,3-diol (apd) headgroup allow for further optimization of overall expression while maintaining or increasing liver selectivity. For this optimization, 6 different aliphatic monomers were selected ranging in length from C6 to C12, including the unsaturated oleyl lipid and branched 2-ethylhexyl. Even with this relatively small monomer pool, there are 55,944 peptoids designs of length 3-8 that are possible, so a systematic approach to evaluating this large structure space using Design of Experiments (DOE) methodology was employed. To allow chemical structure information to be accurately input into a DOE model, the properties of the lipid block can be parametrized using 4 key factors: 1) the total number of lipid-containing monomers in the structure, 2) the total number of carbon atoms contained on all lipid side chains, 3) the number of branched/unsaturated lipid monomers used, and 4) the identity of the branched/unsaturated monomer used. Each peptoid disclosed herein can be represented by a combination of these four factors, and their performance data can be fit to a multivariable model to deconvolute the contribution of each factor to mRNA delivery performance. This method of parameterization does not attempt to capture the order of monomers within the sequence, so peptoids were designed to have alternating and repeating or symmetric motifs wherever possible.

The delivery vehicle compositions of the disclosure comprise hydroxyalkyl-capped cationic peptoids, such as 2-aminopropane-1,3-diol-capped cationic peptoids (“cationic component”, sometimes referred to as an “ionizable lipid”). In some implementations, the hydroxyalkyl-capped cationic peptoids comprise a compound of Formula (I):

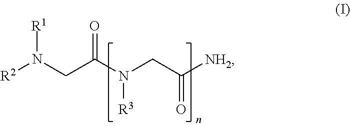

wherein n is n is 1, 2, 3, 4, 5, 6, 7, 8, 9, or 10; R¹ is H or C₂₋₅ alkyloptionally substituted with 1-3 OH; R² is H or C₂₋₅alkylene-OH substituted with 1-3 additional OH; and each R³ independently is C₆₋₂₄alkyl or C₆₋₂₄alkenyl. As used herein, “alkyl” refers to straight chained and branched saturated hydrocarbon groups containing one to thirty carbon atoms, for example, one to twenty four carbon atoms (e.g., 1, 2, 3, 4, 5, 6, 7, 8, 9, 10, 11, 12, 13, 14, 15, 16, 17, 18, 19, 20, 21, 22, 23, or 24 carbon atoms). The term Cₙ means the alkyl group has “n” carbon atoms. For example, C₃ alkyl refers to an alkyl group that has 3 carbon atoms. C₁₋₂₄alkyl refers to an alkyl group having a number of carbon atoms encompassing the entire range (i.e., 1 to 24 carbon atoms), as well as all subgroups (e.g., 1-2, 1-3, 1-4, 1-5, 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 3-4, 3-5, 3-6, 3-7, 3-8, 3- 9, 3-10, 3-11, 3-12, 3-13, 3-14, 3-15, 3-16, 3-17, 3-18, 3-19, 3-20, 3-21, 3-22, 3-23, 3-24, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 5-6, 5-7, 5-8, 5-9, 5-10, 5- 11, 5-12, 5-13, 5-14, 5-15, 5-16, 5-17, 5-18, 5-19, 5-20, 5-21, 5-22, 5-23, 5-24, 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 21-23, 21-24, 22-23, 22-24, 23-24, 1, 2, 3, 4, 5, 6, 7, 8, 9, 10, 11, 12, 13, 14, 15, 16, 17, 18, 19, 20, 21, 22, 23, or 24 carbon atoms). Nonlimiting examples of alkyl groups include, methyl, ethyl, n-propyl, isopropyl, n-butyl, sec-butyl (2-methylpropyl), and t-butyl (1,1-dimethylethyl). Unless otherwise indicated, an alkyl group can be an unsubstituted alkyl group or a substituted alkyl group. As used herein, “alkenyl” refers to straight chained and branched hydrocarbon groups having a double bond and containing two to thirty carbon atoms, for example, two to twenty four carbon atoms (e.g., 1, 2, 3, 4, 5, 6, 7, 8, 9, 10, 11, 12, 13, 14, 15, 16, 17, 18, 19, 20, 21, 22, 23, or 24 carbon atoms). The term Cₙ means the alkenyl group has “n” carbon atoms. For example, C₃ alkenyl refers to an alkenyl group that has 3 carbon atoms. C₂-C₂₄ alkenyl refers to an alkenyl group having a number of carbon atoms encompassing the entire range (i.e., 2 to 24 carbon atoms), as well as all subgroups (e.g., 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 5-6, 5-7, 5-8, 5-9, 5-10, 5-11, 5-12, 5-13, 5-14, 5-15, 5-16, 5-17, 5-18, 5-19, 5-20, 5- 21, 5-22, 5-23, 5-24, 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 7-8, 7-9, 7-10, 7-11, 7-12, 7-13, 7-14, 7-15, 7-16, 7-17, 7-18, 7-19, 7-20, 7-21, 7-22, 7-23, 7-24, 8-9, 8-10, 8-11, 8-12, 8-13, 8-14, 8-15, 8-16, 8-17, 8-18, 8-19, 8-20, 8-21, 8-22, 8-23, 8-24, 9-10, 9-11, 9-12, 9-13, 9-14, 9-15, 9-16, 9-17, 9-18, 9-19, 9-20, 9-21, 9-22, 9-23, 9-24, 10-11, 10-12, 10-13, 10-14, 10-15, 10-16, 10-17, 10-18, 10-19, 10-20, 10-21, 10-22, 10-23, 10-24, 11-12, 11-13, 11-14, 11-15, 11-16, 11-17, 11-18, 11-19, 11-20, 11-21, 11-22, 11-23, 11-24, 12-13, 12-14, 12-15, 12-16, 12-17, 12-18, 12-19, 12-20, 12-21, 12-22, 12-23, 12-24, 13-14, 13-15, 13-16, 13-17, 13-18, 13-19, 13-20, 13-21, 13-22, 13-23, 13-24, 14-15, 14-16, 14-17, 14-18, 14-19, 14-20, 14-21, 14-22, 14-23, 14-24, 15-16, 15-17, 15-18, 15-19, 15-20, 15-21, 15-22, 15-23, 15-24, 16-17, 16-18, 16-19, 16-20, 16-21, 16-22, 16-23, 16-24, 17-18, 17-19, 17-20, 17-21, 17-22, 17-23, 17-24, 18-19, 18-20, 18-21, 18-22, 18-23, 18-24, 19-20, 19-21, 19-22, 19-23, 19-24, 20-21, 20-22, 20-23, 20-24, 21-22, 21-23, 21-24, 22-23, 22-24, 23-24, 1, 2, 3, 4, 5, 6, 7, 8, 9, 10, 11, 12, 13, 14, 15, 16, 17, 18, 19, 20, 21, 22, 23, or 24 carbon atoms). Nonlimiting examples of alkenyl groups include ethenyl, propenyl, butenyl, geranyl, and oleyl. Unless otherwise indicated, an alkenyl group can be an unsubstituted alkenyl group or a substituted alkenyl group.

In some implementations, n is 2 to 5. In various implementations, n is 3 to 4. In some implementations, n is 1. In various implementations, n is 2. In some cases, n is 3. In various cases, n is 4. In some implementations n is 5. In various implementations, n is 6. In various cases, n is 7. In various cases, n is 8. In various cases, n is 9. In various cases, n is 10.

In some implementations, R¹ is H. In various implementations, R¹ is C₂₋₅alkyl optionally substituted with 1-3 OH. In some cases, R¹ is methyl or ethyl. In some implementations, R¹ is ethyl. In various implementations, R¹ is C₂₋₅alkylene-OH substituted with 0-2 additional OH. In some cases, R¹ is

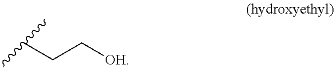 [SMILES: *CCO]

In various cases, R¹ is ethyl or hydroxyethyl. In some cases, C₂₋₅alkyl is substituted with 1 OH. In some cases, C₂₋₅alkyl is substituted with 2 OH. In some cases, C₂₋₅alkyl is substituted with 3 OH.

R² is C₂₋₅alkylene-OH substituted with 1-3 additional OH. In some cases, R² is C₂alkylene-OH substituted with 1-3 additional OH. In some cases, R² is C₃alkylene-OH substituted with 1-3 additinoal OH. In some cases, R² is C₄alkylene-OH substituted with 1-3 additional OH. In some cases, R² is C₃alkylene-OH substituted with 1-3 additional OH. In some cases, C₂₋₅alkylene-OH is substituted with 1 additional OH. In some cases, C₂₋₅alkylene-OH is substituted with 2 additional OH. In some cases, C₂₋₅alkylene-OH is substituted with 3 additional OH. In some cases, R² is propyl-1,3-diol. In some cases, R² is

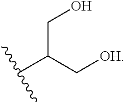 [SMILES: *C(CO)CO]

In some implementations, each R³ independently is C₈₋₁₈alkyl or C₈₋₁₈alkenyl. In various implementations, each R³ independently is C₈₋₁₆alkyl or C₁₀₋₁₈alkenyl. In some cases, each R³ independently is C₆₋₁₈alkyl or C₆₋₁₈alkenyl. In some cases, each R³ independently is C₁₀₋₁₂alkyl or C₁₀₋₁₈alkenyl. In some implementations, each R³ independently is: C₈₋₁₈alkyl, or C₈₋₁₆alkyl, or C₈₋₁₄alkyl, or C₈₋₁₂alkyl. In various implementations, each R³ independently is selected from the group consisting of

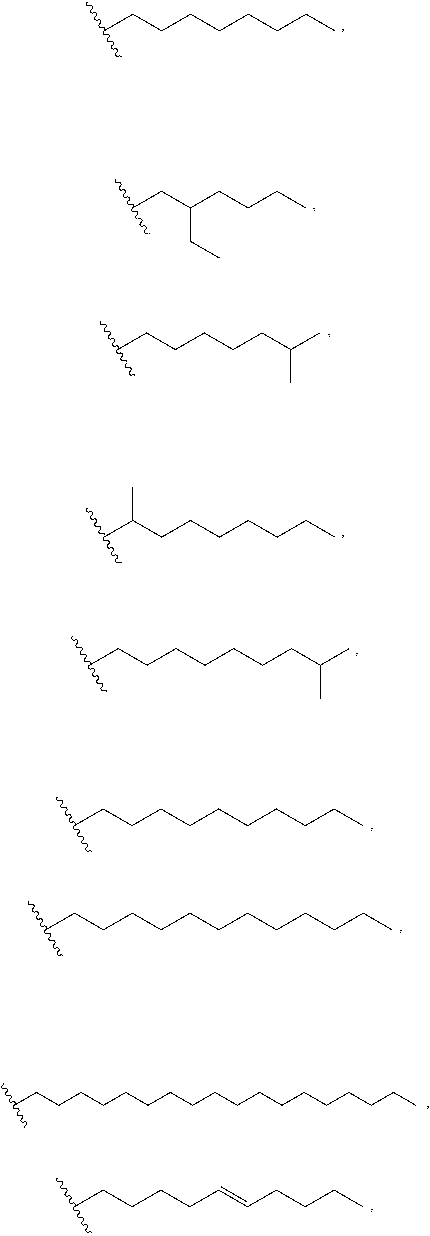

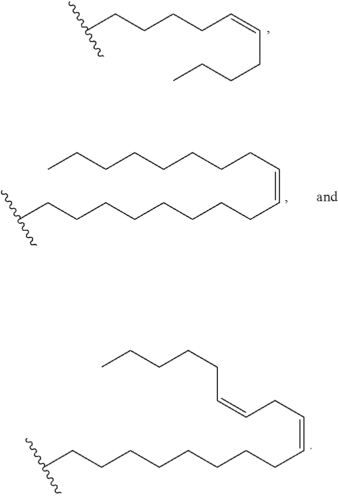 [SMILES: *CCCC/C=C\CCCC ; *CCCCCCCC/C=C\CCCCCCCC ; *CCCCCCCC/C=C\C/C=C\CCCCC]

In some cases, each R3 independently is selected from the group consisting of

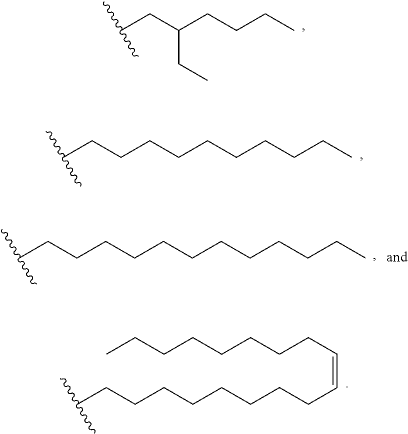

In various cases, each R³ independently is selected from the group consisting of

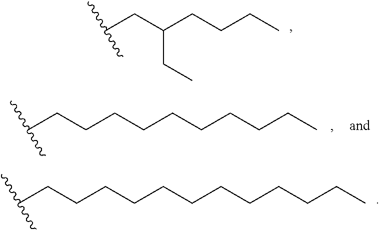

In some implementations, each R³ independently is

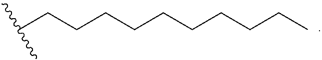 [SMILES: *CCCCCCCCCC]

Contemplated compounds of Formula (I) include, but are not limited to, the compounds listed in Table 1.

TABLE 1
Examples of hydroxyalkyl-capped cationic peptoids.

| Com- | |
|---|---|
| pound | Structure |
| 1 | 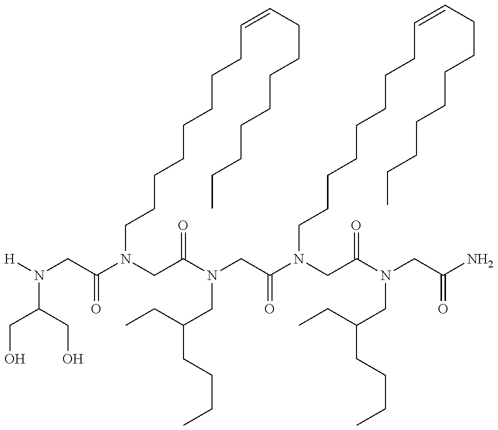 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNC(CO)CO] |
| 2 | 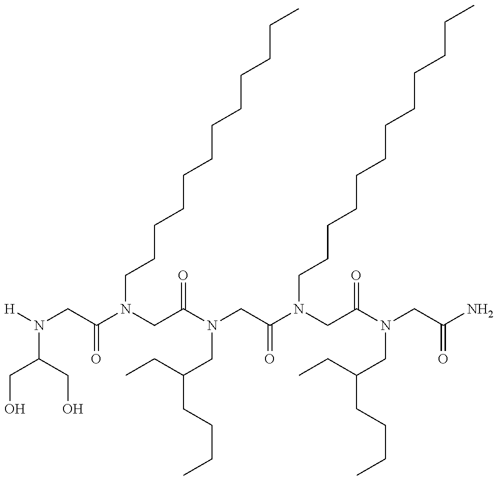 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 3 | 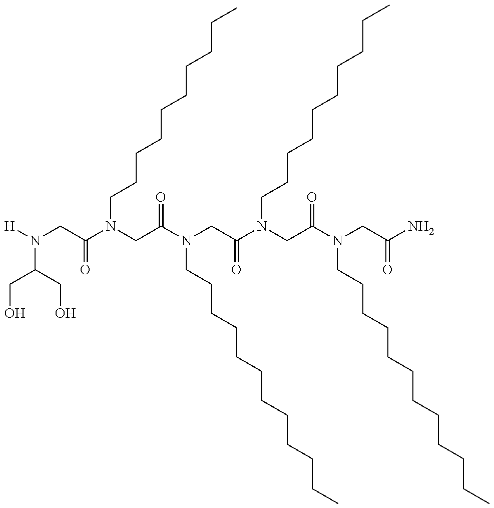 [SMILES: CCCCCCCCCCCCN(CC(N)=O)C(=O)CN(CCCCCCCCCC)C(=O)CN(CCCCCCCCCCCC)C(=O)CN(CCCCCCCCCC)C(=O)CNC(CO)CO] |
| 4 | 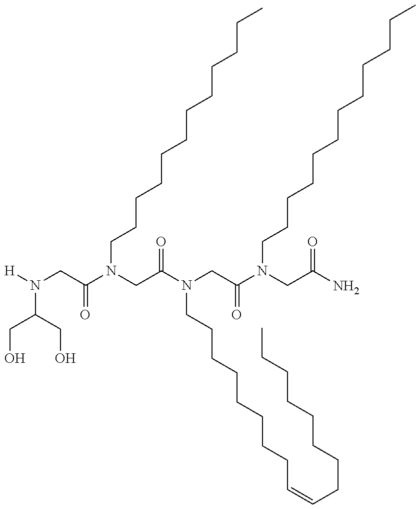 [SMILES: CCCCCCCC/C=C\CCCCCCCCN(CC(=O)N(CCCCCCCCCCCC)CC(N)=O)C(=O)CN(CCCCCCCCCCCC)C(=O)CNC(CO)CO] |
| 5 | 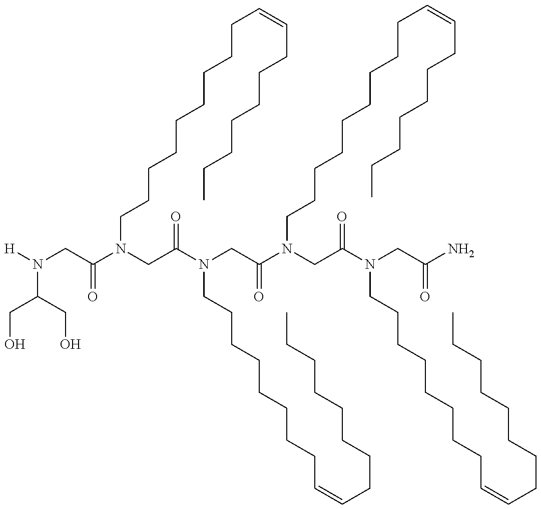 [SMILES: CCCCCCC/C=C\CCCCCCCCCN(CC(=O)N(CCCCCCCC/C=C\CCCCCCCC)CC(=O)N(CCCCCCCCC/C=C\CCCCCCC)CC(=O)N(CCCCCCCC/C=C\CCCCCCCC)CC(N)=O)C(=O)CNC(CO)CO] |
| 6 | 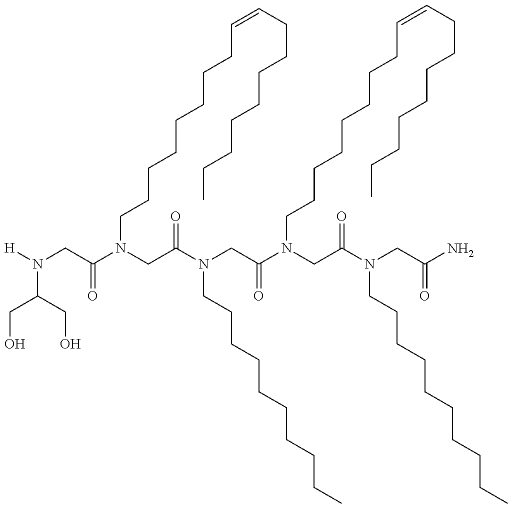 [SMILES: CCCCCCCC/C=C\CCCCCCCCN(CC(=O)N(CCCCCCCCCC)CC(=O)N(CCCCCCCC/C=C\CCCCCCCC)CC(=O)N(CCCCCCCCCC)CC(N)=O)C(=O)CNC(CO)CO] |
| 7 | 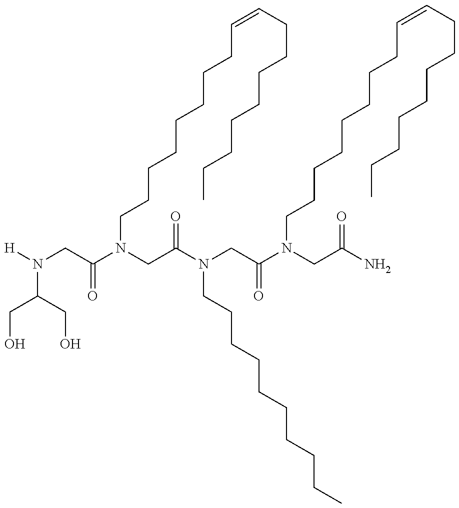 [SMILES: CCCCCCCC/C=C\CCCCCCCCN(CC(N)=O)C(=O)CN(CCCCCCCCCC)C(=O)CN(CCCCCCCC/C=C\CCCCCCCC)C(=O)CNC(CO)CO] |
| 8 | 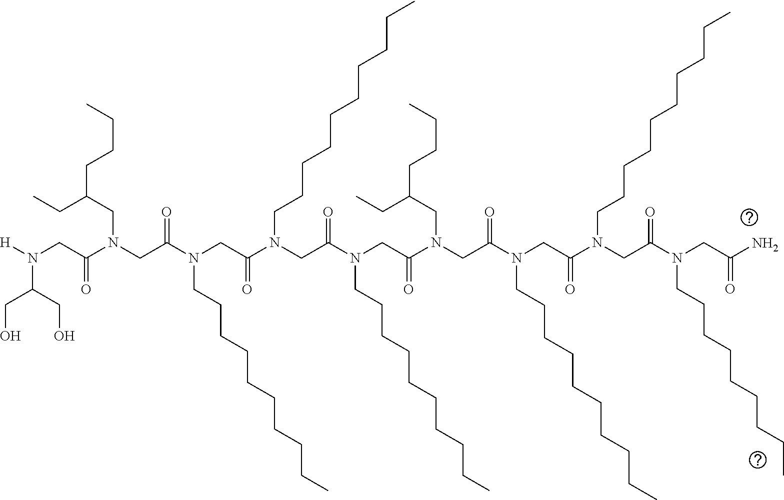 [SMILES: CCCCCCCCCCN(CC(=O)N(CCCCCCCCC)CC(N)=O)C(=O)CN(CCCCCCCCCC)C(=O)CN(CC(CC)CCCC)C(=O)CN(CCCCCCCCCC)C(=O)CN(CCCCCCCCCC)C(=O)CN(CCCCCCCCCC)C(=O)CN(CC(CC)CCCC)C(=O)CNC(CO)CO] |
| 9 | 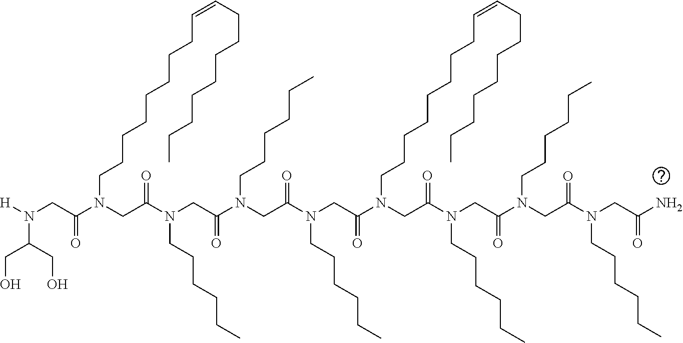 [SMILES: CCCCCCCC/C=C\CCCCCCCCN(CC(=O)N(CCCCCC)CC(=O)N(CCCCCC)CC(=O)N(CCCCCC)CC(=O)N(CCCCCCCC/C=C\CCCCCCCC)CC(=O)N(CCCCCC)CC(=O)N(CCCCCC)CC(=O)N(CCCCCC)CC(N)=O)C(=O)CNC(CO)CO] |
| 10 | 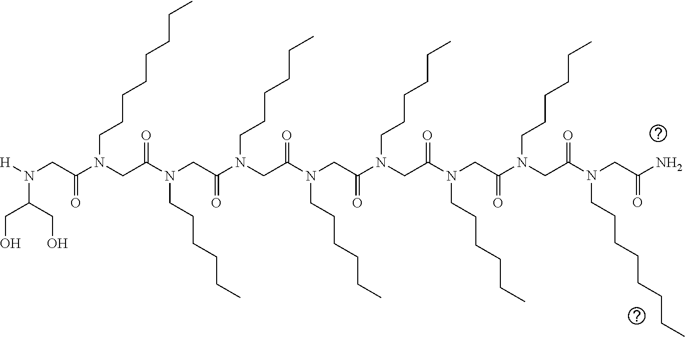 [SMILES: CCCCCCCCN(CC(N)=O)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CN(CCCCCCCC)C(=O)CNC(CO)CO] |
| 11 | 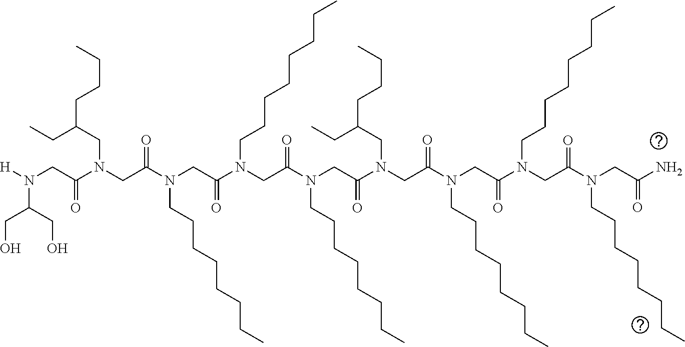 [SMILES: CCCCCCCCN(CC(N)=O)C(=O)CN(CCCCCCCC)C(=O)CN(CCCCCCCC)C(=O)CN(CC(CC)CCCC)C(=O)CN(CCCCCCCC)C(=O)CN(CCCCCCCC)C(=O)CN(CCCCCCCC)C(=O)CN(CC(CC)CCCC)C(=O)CNC(CO)CO] |
| 12 | 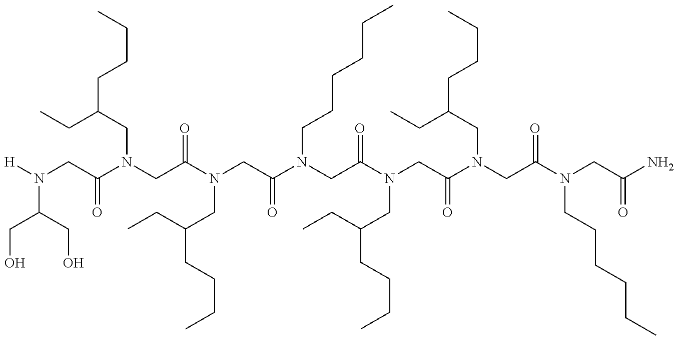 [SMILES: CCCCCCN(CC(N)=O)C(=O)CN(CC(CC)CCCC)C(=O)CN(CC(CC)CCCC)C(=O)CN(CCCCCC)C(=O)CN(CC(CC)CCCC)C(=O)CN(CC(CC)CCCC)C(=O)CNC(CO)CO] |
| 13 | 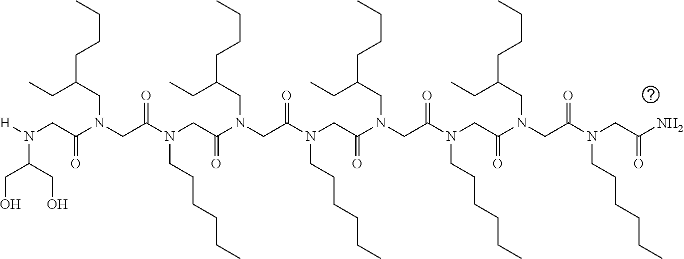 [SMILES: CCCCCCN(CC(N)=O)C(=O)CN(CC(CC)CCCC)C(=O)CN(CCCCCC)C(=O)CN(CC(CC)CCCC)C(=O)CN(CCCCCC)C(=O)CN(CC(CC)CCCC)C(=O)CN(CCCCCC)C(=O)CN(CC(CC)CCCC)C(=O)CNC(CO)CO] |
| 14 | 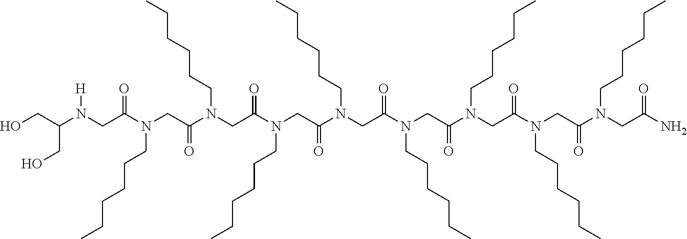 [SMILES: CCCCCCN(CC(N)=O)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CNC(CO)CO] |
| 15 | 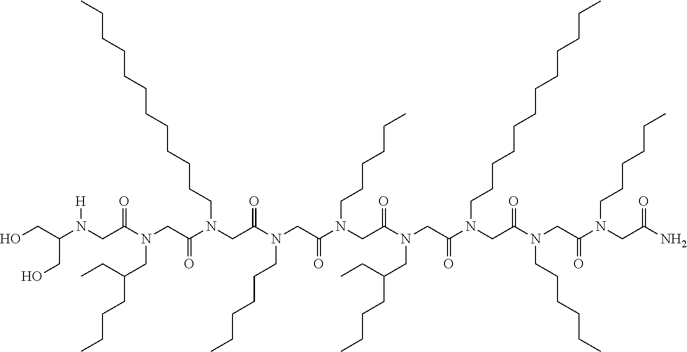 [SMILES: CCCCCCCCCCCCN(CC(=O)N(CCCCCC)CC(=O)N(CCCCCC)CC(=O)N(CC(=O)N(CCCCCCCCCCCC)CC(=O)N(CCCCCC)CC(=O)N(CCCCCC)CC(N)=O)CC(CC)CCCC)C(=O)CN(CC(CC)CCCC)C(=O)CNC(CO)CO] |
| 16 | 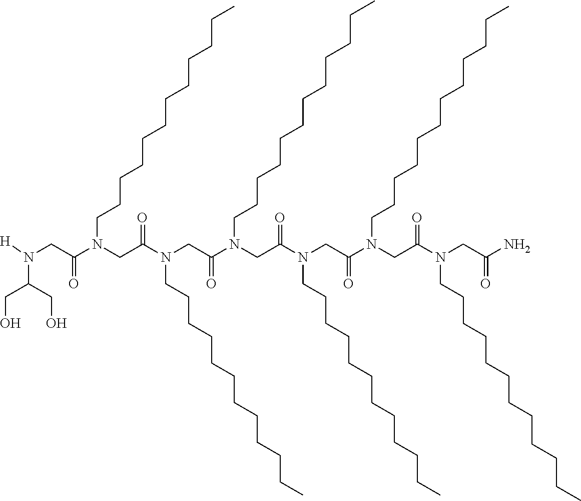 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNC(CO)CO] |
| 17 | 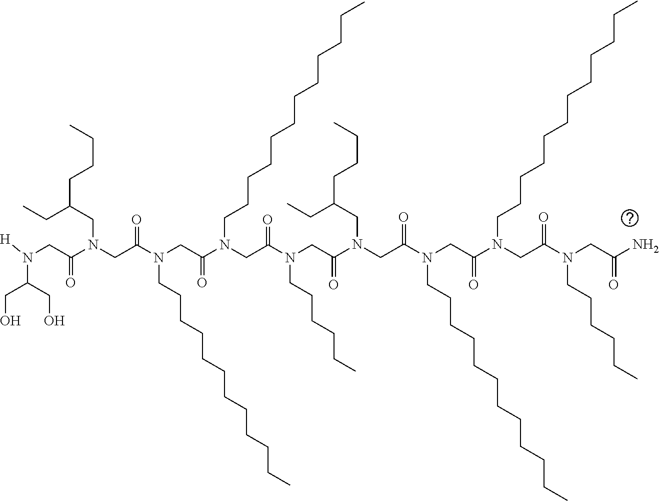 [SMILES: CCCCCCCCCCCCN(CC(=O)N(CCCCCC)CC(N)=O)C(=O)CN(CCCCCCCCCCCC)C(=O)CN(CC(CC)CCCC)C(=O)CN(CCCCCC)C(=O)CN(CCCCCCCCCCCC)C(=O)CN(CCCCCCCCCCCC)C(=O)CN(CC(CC)CCCC)C(=O)CNC(CO)CO] |
| 18 | 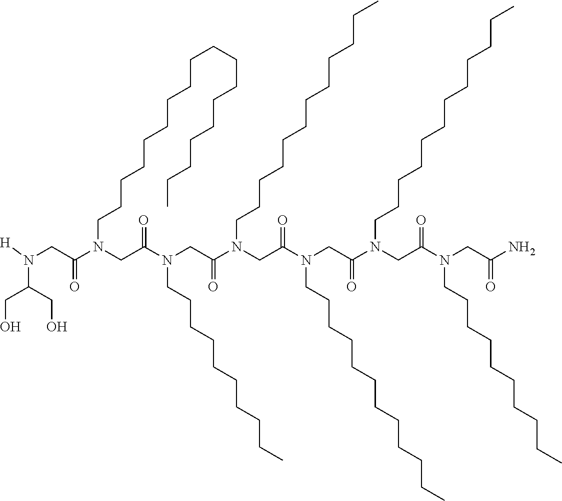 [SMILES: CCCCCCCCCCCCCCCCCCN(CC(=O)N(CCCCCCCCCC)CC(=O)N(CCCCCCCCCCCC)CC(=O)N(CCCCCCCCCCCC)CC(=O)N(CCCCCCCCCCCC)CC(=O)N(CCCCCCCCCC)CC(N)=O)C(=O)CNC(CO)CO] |
| 19 | 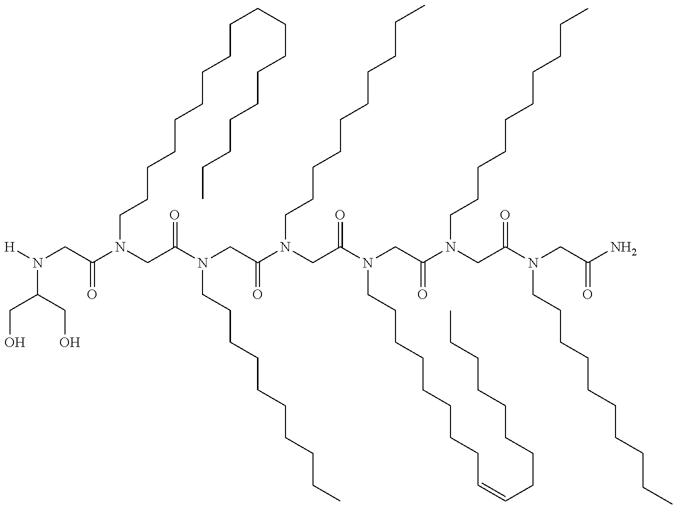 [SMILES: CCCCCCCC/C=C\CCCCCCCCN(CC(=O)N(CCCCCCCCCC)CC(=O)N(CCCCCCCCCC)CC(N)=O)C(=O)CN(CCCCCCCCCC)C(=O)CN(CCCCCCCCCC)C(=O)CN(CCCCCCCCCCCCCCCCCC)C(=O)CNC(CO)CO] |
| 20 | 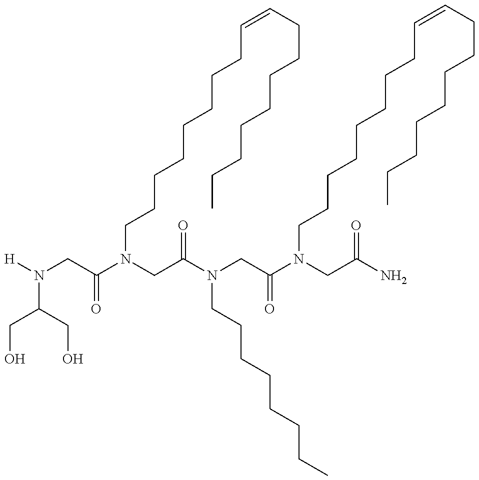 [SMILES: CCCCCCCC/C=C\CCCCCCCCN(CC(N)=O)C(=O)CN(CCCCCCCC)C(=O)CN(CCCCCCCC/C=C\CCCCCCCC)C(=O)CNC(CO)CO] |
| 21 | 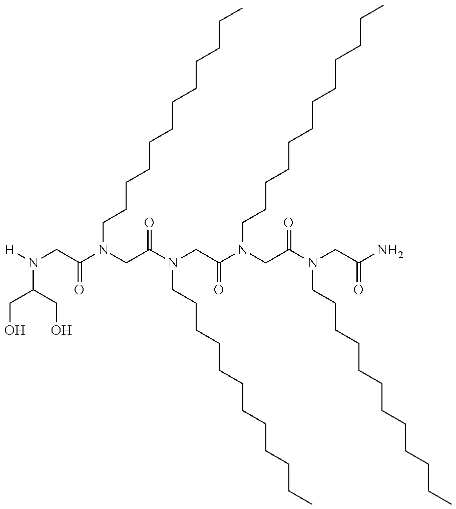 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNC(CO)CO] |
| 22 | 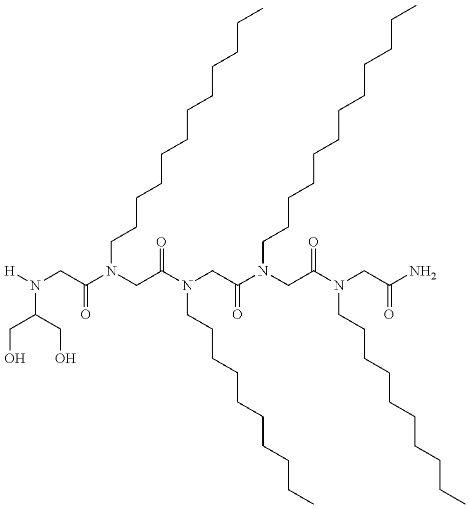 [SMILES: CCCCCCCCCCCCN(CC(=O)N(CCCCCCCCCC)CC(=O)N(CCCCCCCCCCCC)CC(=O)N(CCCCCCCCCC)CC(N)=O)C(=O)CNC(CO)CO] |
| 23 | 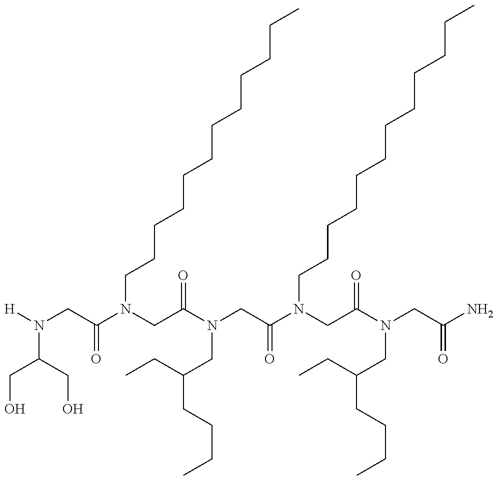 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 24 | 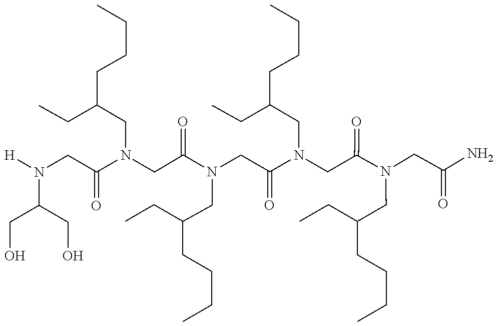 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CNC(CO)CO] |
| 25 | 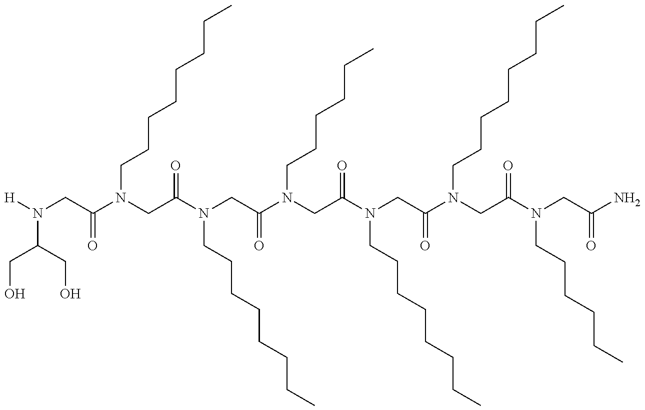 [SMILES: CCCCCCCCN(CC(=O)N(CCCCCCCC)CC(=O)N(CCCCCC)CC(=O)N(CCCCCCCC)CC(=O)N(CCCCCCCC)CC(=O)N(CCCCCC)CC(N)=O)C(=O)CNC(CO)CO] |
| 26 | 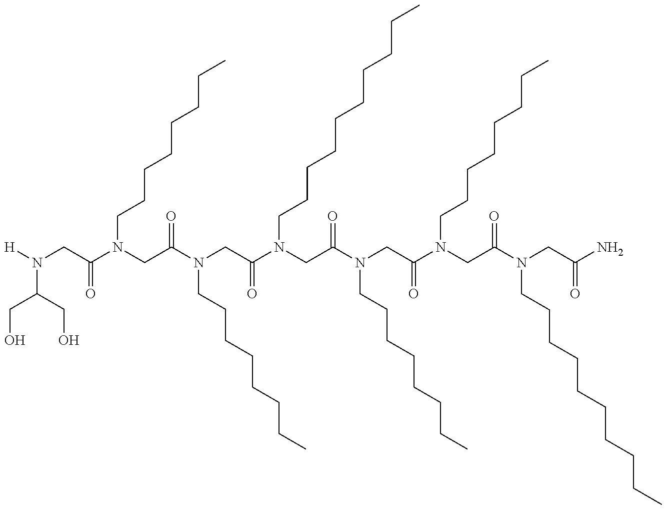 [SMILES: CCCCCCCCCCN(CC(N)=O)C(=O)CN(CCCCCCCC)C(=O)CN(CCCCCCCC)C(=O)CN(CCCCCCCCCC)C(=O)CN(CCCCCCCC)C(=O)CN(CCCCCCCC)C(=O)CNC(CO)CO] |
| 27 | 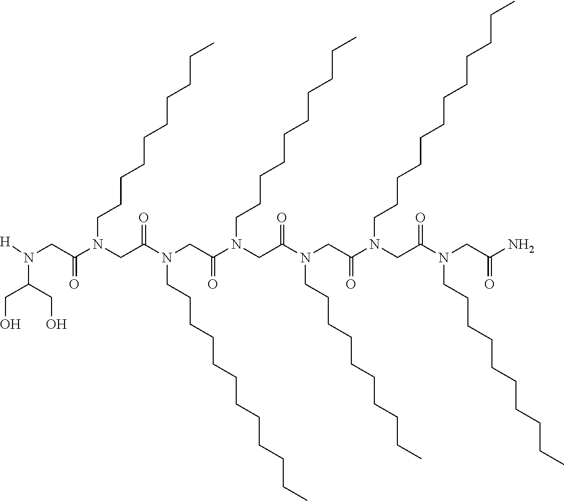 [SMILES: CCCCCCCCCCCCN(CC(=O)N(CCCCCCCCCC)CC(N)=O)C(=O)CN(CCCCCCCCCC)C(=O)CN(CCCCCCCCCC)C(=O)CN(CCCCCCCCCCCC)C(=O)CN(CCCCCCCCCC)C(=O)CNC(CO)CO] |
| 28 | 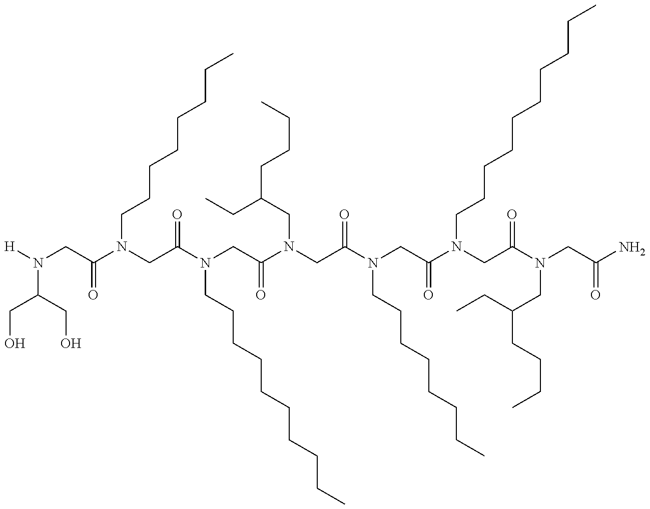 [SMILES: CCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCC)C(=O)CN(CCCCCCCC)C(=O)CNC(CO)CO] |
| 29 | 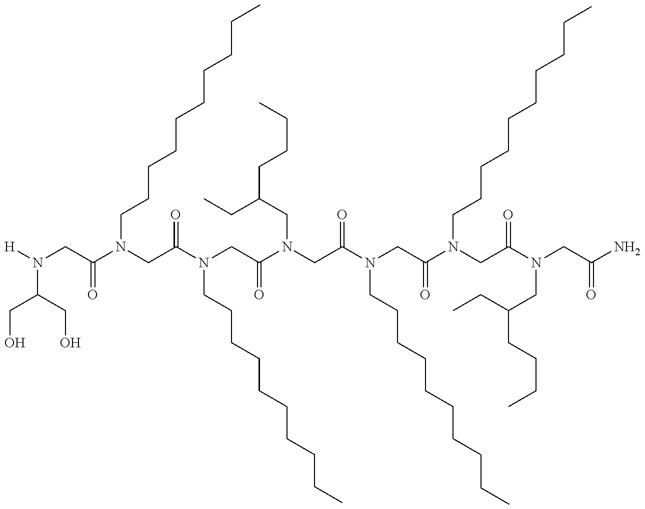 [SMILES: CCCCCCCCCCN(CC(=O)N(CCCCCCCCCC)CC(=O)N(CC(=O)N(CCCCCCCCCC)CC(=O)N(CCCCCCCCCC)CC(=O)N(CC(N)=O)CC(CC)CCCC)CC(CC)CCCC)C(=O)CNC(CO)CO] |
| 30 | 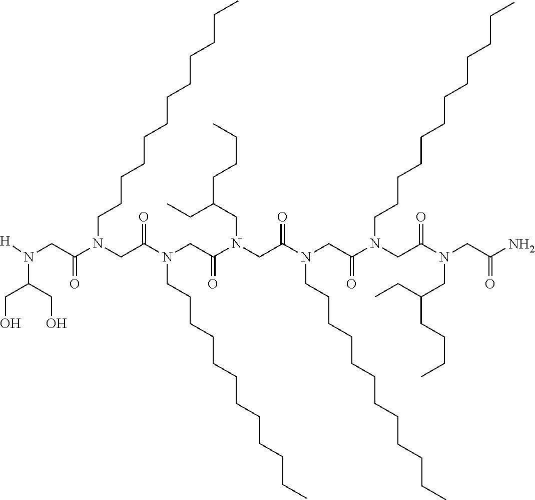 [SMILES: CCCCCCCCCCCCN(CC(=O)N(CCCCCCCCCCCC)CC(=O)N(CC(=O)N(CCCCCCCCCCCC)CC(=O)N(CCCCCCCCCCCC)CC(=O)N(CC(N)=O)CC(CC)CCCC)CC(CC)CCCC)C(=O)CNC(CO)CO] |
| 31 | 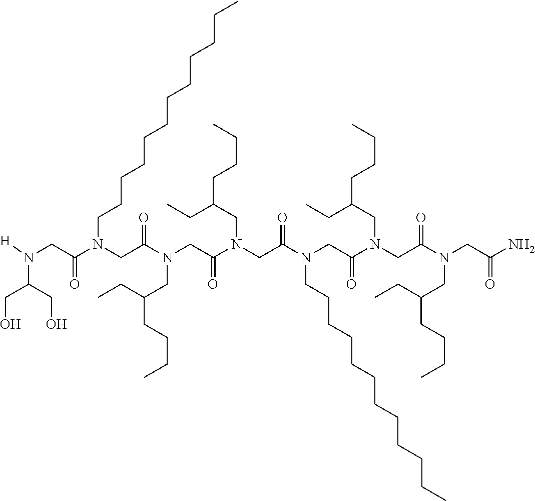 [SMILES: CCCCCCCCCCCCN(CC(=O)N(CC(=O)N(CC(=O)N(CCCCCCCCCCCC)CC(=O)N(CC(=O)N(CC(N)=O)CC(CC)CCCC)CC(CC)CCCC)CC(CC)CCCC)CC(CC)CCCC)C(=O)CNC(CO)CO] |
| 32 | 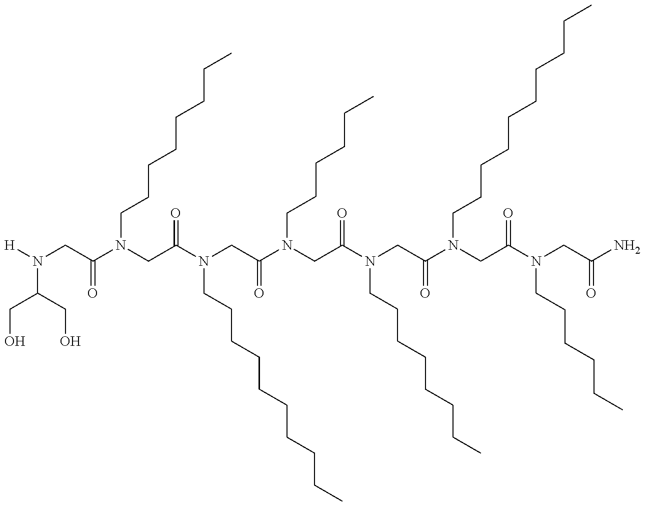 [SMILES: CCCCCCCCCCN(CC(=O)N(CCCCCC)CC(N)=O)C(=O)CN(CCCCCCCC)C(=O)CN(CCCCCC)C(=O)CN(CCCCCCCCCC)C(=O)CN(CCCCCCCC)C(=O)CNC(CO)CO] |
| 33 | 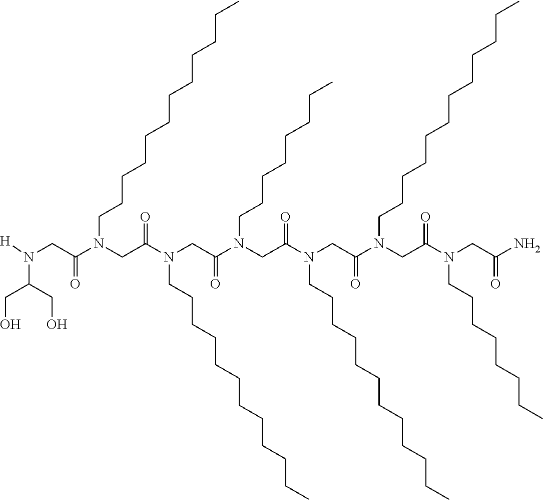 [SMILES: CCCCCCCCCCCCN(CC(=O)N(CCCCCCCCCCCC)CC(=O)N(CCCCCCCC)CC(=O)N(CCCCCCCCCCCC)CC(=O)N(CCCCCCCCCCCC)CC(=O)N(CCCCCCCC)CC(N)=O)C(=O)CNC(CO)CO] |
| 34 | 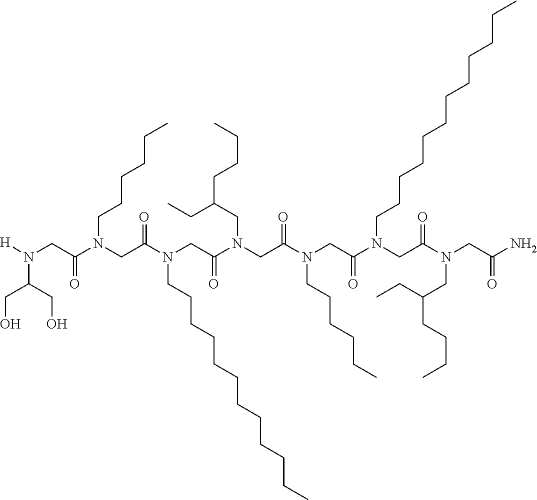 [SMILES: CCCCCCCCCCCCN(CC(=O)N(CC(N)=O)CC(CC)CCCC)C(=O)CN(CCCCCC)C(=O)CN(CC(CC)CCCC)C(=O)CN(CCCCCCCCCCCC)C(=O)CN(CCCCCC)C(=O)CNC(CO)CO] |
| 35 | 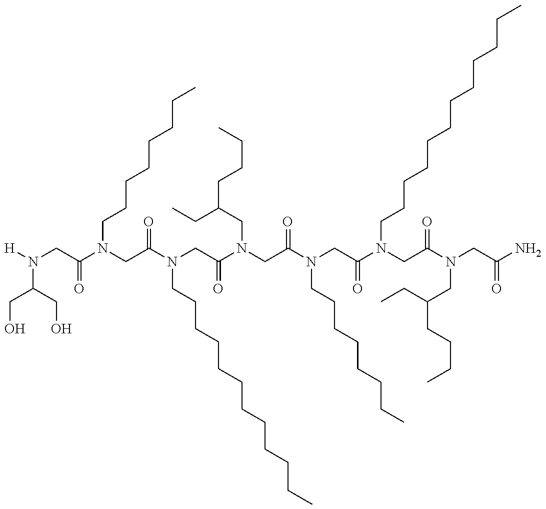 [SMILES: CCCCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCCCC)C(=O)CN(CCCCCCCC)C(=O)CNC(CO)CO] |
| 36 | 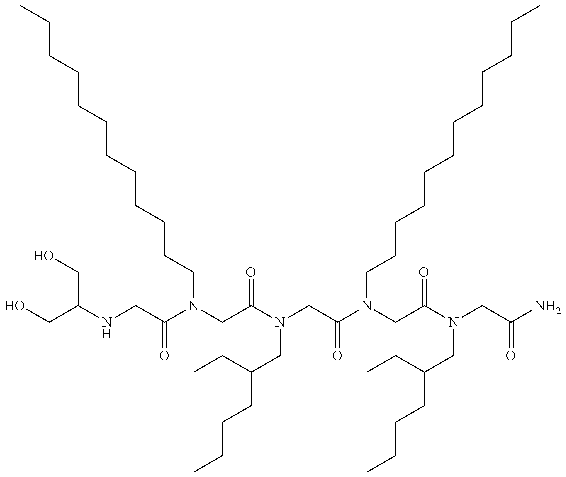 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 37 | 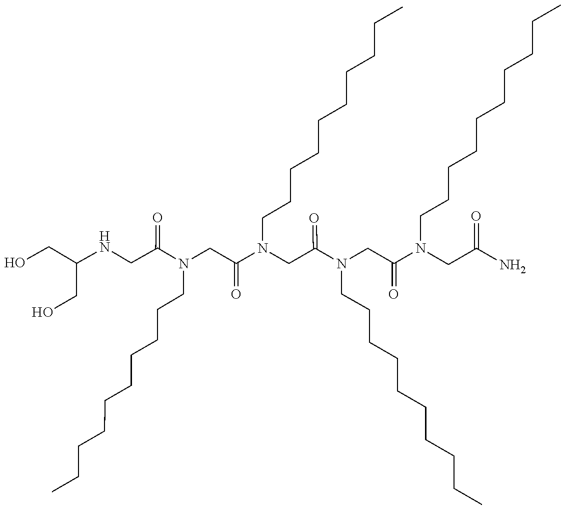 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNC(CO)CO] |
| 40 | 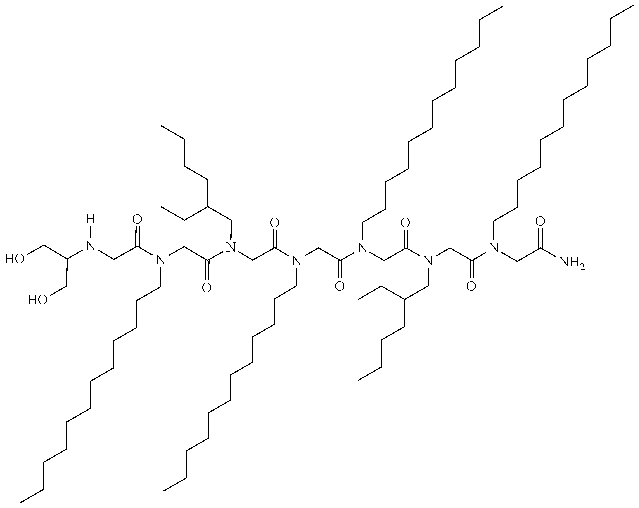 [SMILES: CCCCCCCCCCCCN(CC(N)=O)C(=O)CN(CC(CC)CCCC)C(=O)CN(CCCCCCCCCCCC)C(=O)CN(CCCCCCCCCCCC)C(=O)CN(CC(CC)CCCC)C(=O)CN(CCCCCCCCCCCC)C(=O)CNC(CO)CO] |
| 41 | 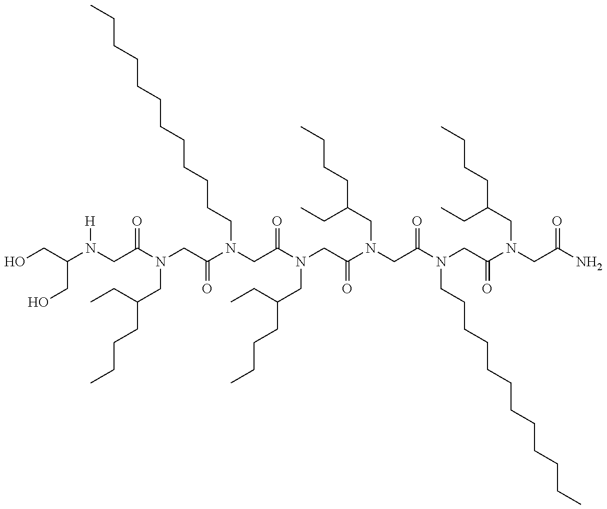 [SMILES: CCCCCCCCCCCCN(CC(=O)N(CC(=O)N(CC(=O)N(CCCCCCCCCCCC)CC(=O)N(CC(N)=O)CC(CC)CCCC)CC(CC)CCCC)CC(CC)CCCC)C(=O)CN(CC(CC)CCCC)C(=O)CNC(CO)CO] |
| 42 | 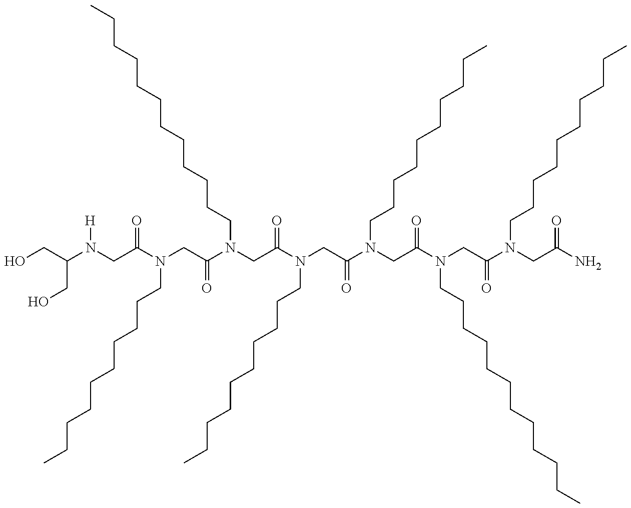 [SMILES: CCCCCCCCCCCCN(CC(=O)N(CCCCCCCCCC)CC(N)=O)C(=O)CN(CCCCCCCCCC)C(=O)CN(CCCCCCCCCC)C(=O)CN(CCCCCCCCCCCC)C(=O)CN(CCCCCCCCCC)C(=O)CNC(CO)CO] |
| 43 | 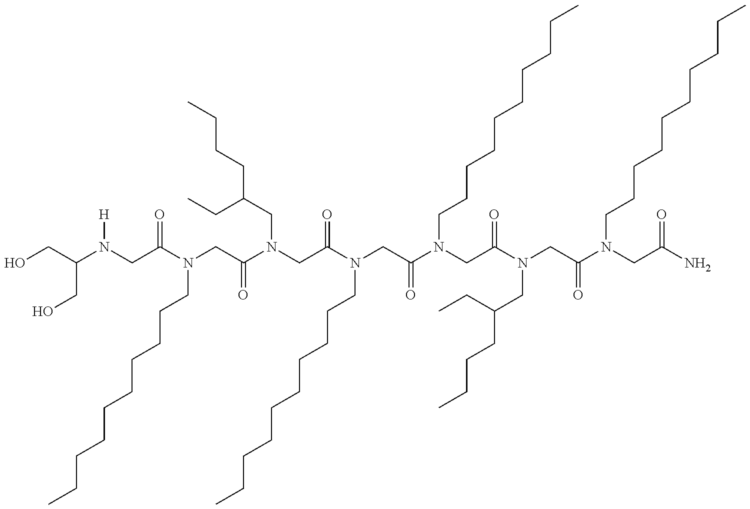 [SMILES: CCCCCCCCCCN(CC(N)=O)C(=O)CN(CC(CC)CCCC)C(=O)CN(CCCCCCCCCC)C(=O)CN(CCCCCCCCCC)C(=O)CN(CC(CC)CCCC)C(=O)CN(CCCCCCCCCC)C(=O)CNC(CO)CO] |
| 44 | 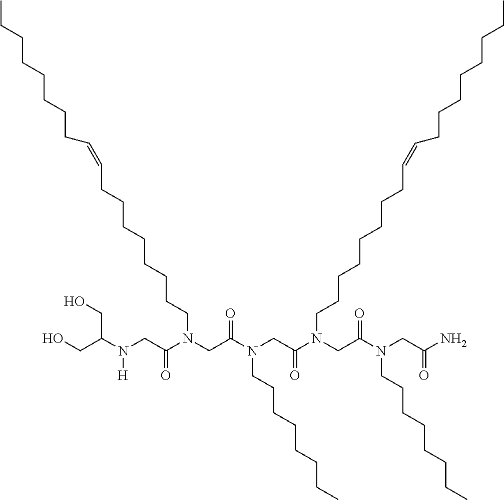 [SMILES: CCCCCCCC/C=C\CCCCCCCCN(CC(=O)N(CCCCCCCC)CC(=O)N(CCCCCCCC/C=C\CCCCCCCC)CC(=O)N(CCCCCCCC)CC(N)=O)C(=O)CNC(CO)CO] |
| 45 | 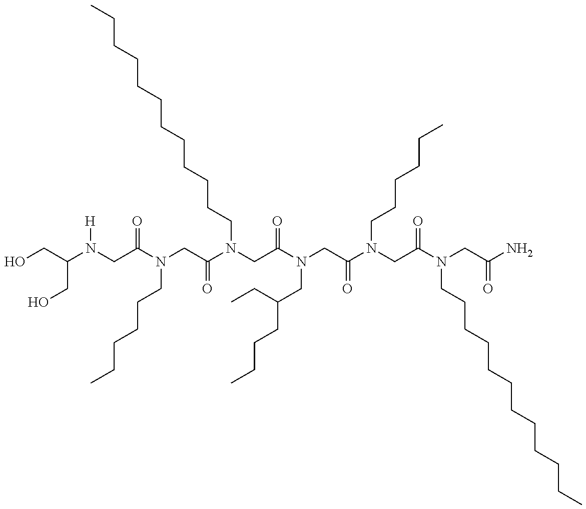 [SMILES: CCCCCCCCCCCCN(CC(N)=O)C(=O)CN(CCCCCC)C(=O)CN(CC(CC)CCCC)C(=O)CN(CCCCCCCCCCCC)C(=O)CN(CCCCCC)C(=O)CNC(CO)CO] |
| 46 | 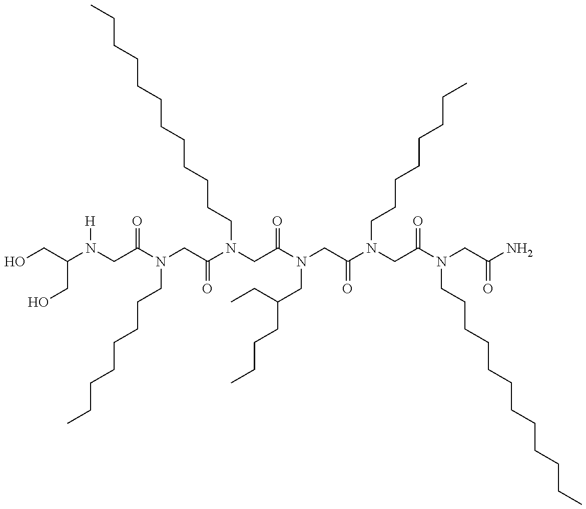 [SMILES: CCCCCCCCCCCCN(CC(N)=O)C(=O)CN(CCCCCCCC)C(=O)CN(CC(CC)CCCC)C(=O)CN(CCCCCCCCCCCC)C(=O)CN(CCCCCCCC)C(=O)CNC(CO)CO] |
| 47 | 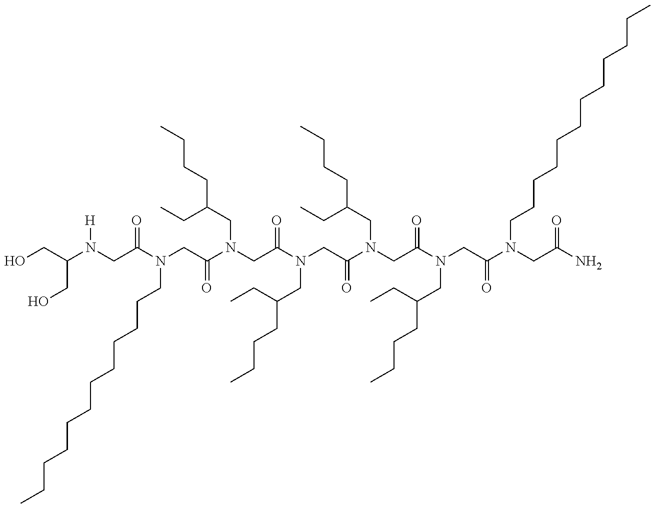 [SMILES: CCCCCCCCCCCCN(CC(N)=O)C(=O)CN(CC(CC)CCCC)C(=O)CN(CC(CC)CCCC)C(=O)CN(CC(CC)CCCC)C(=O)CN(CC(CC)CCCC)C(=O)CN(CCCCCCCCCCCC)C(=O)CNC(CO)CO] |
| 48 | 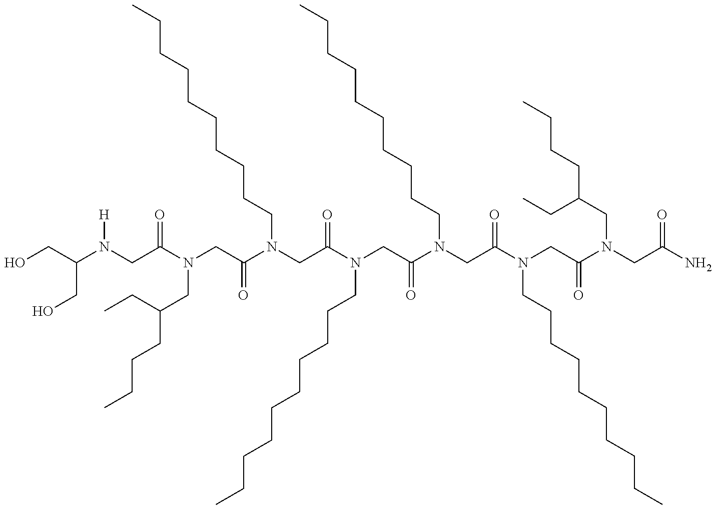 [SMILES: CCCCCCCCCCN(CC(=O)N(CCCCCCCCCC)CC(=O)N(CC(N)=O)CC(CC)CCCC)C(=O)CN(CCCCCCCCCC)C(=O)CN(CCCCCCCCCC)C(=O)CN(CC(CC)CCCC)C(=O)CNC(CO)CO] |
| 49 | 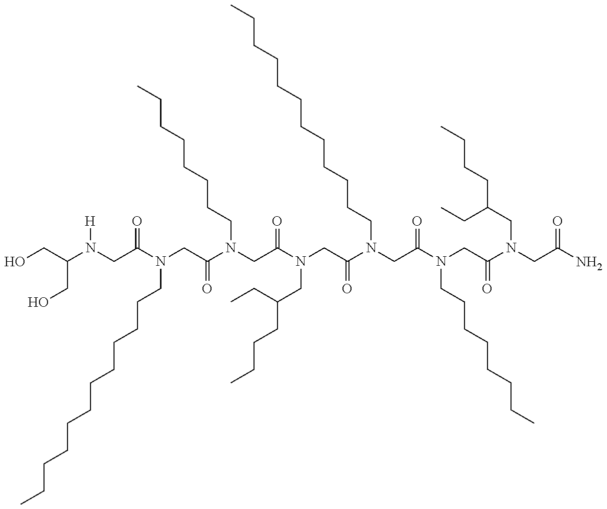 [SMILES: CCCCCCCCCCCCN(CC(=O)N(CCCCCCCC)CC(=O)N(CC(=O)N(CCCCCCCCCCCC)CC(=O)N(CCCCCCCC)CC(=O)N(CC(N)=O)CC(CC)CCCC)CC(CC)CCCC)C(=O)CNC(CO)CO] |
| 51 | 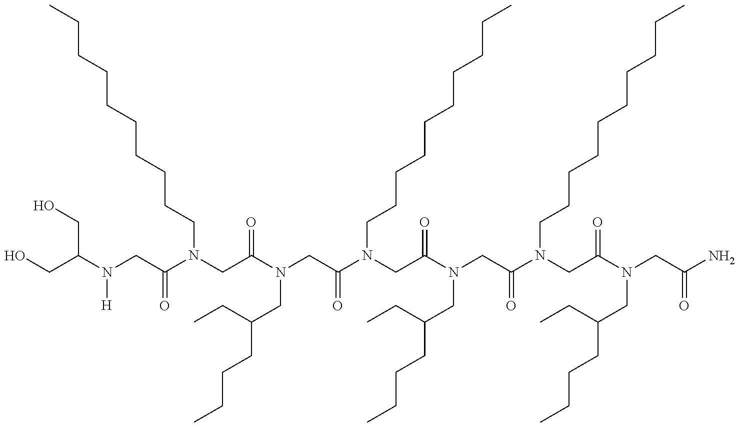 [SMILES: CCCCCCCCCCN(CC(=O)N(CC(=O)N(CCCCCCCCCC)CC(=O)N(CC(=O)N(CCCCCCCCCC)CC(=O)N(CC(N)=O)CC(CC)CCCC)CC(CC)CCCC)CC(CC)CCCC)C(=O)CNC(CO)CO] |
| 52 | 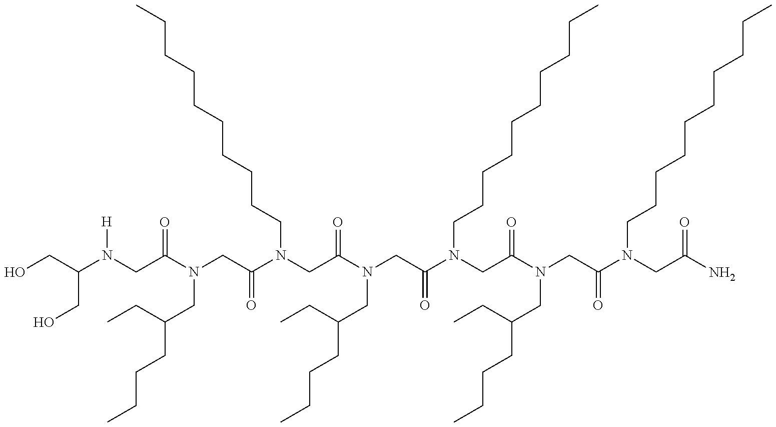 [SMILES: CCCCCCCCCCN(CC(N)=O)C(=O)CN(CC(CC)CCCC)C(=O)CN(CCCCCCCCCC)C(=O)CN(CC(CC)CCCC)C(=O)CN(CCCCCCCCCC)C(=O)CN(CC(CC)CCCC)C(=O)CNC(CO)CO] |
| 53 | 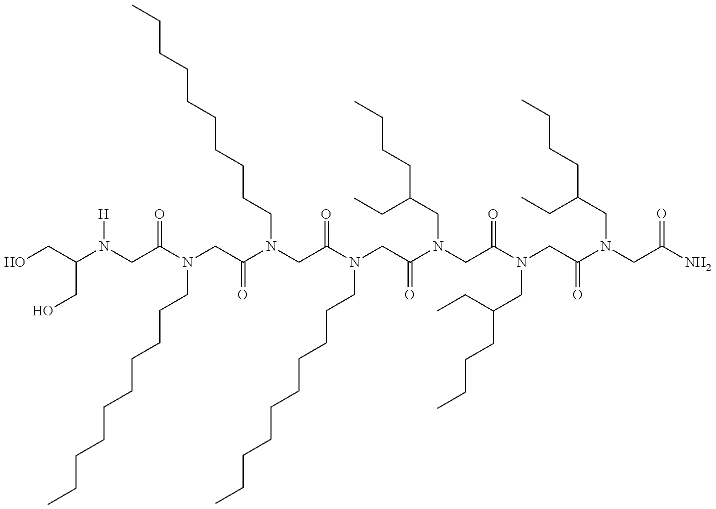 [SMILES: CCCCCCCCCCN(CC(=O)N(CCCCCCCCCC)CC(=O)N(CCCCCCCCCC)CC(=O)N(CC(=O)N(CC(=O)N(CC(N)=O)CC(CC)CCCC)CC(CC)CCCC)CC(CC)CCCC)C(=O)CNC(CO)CO] |
| 54 | 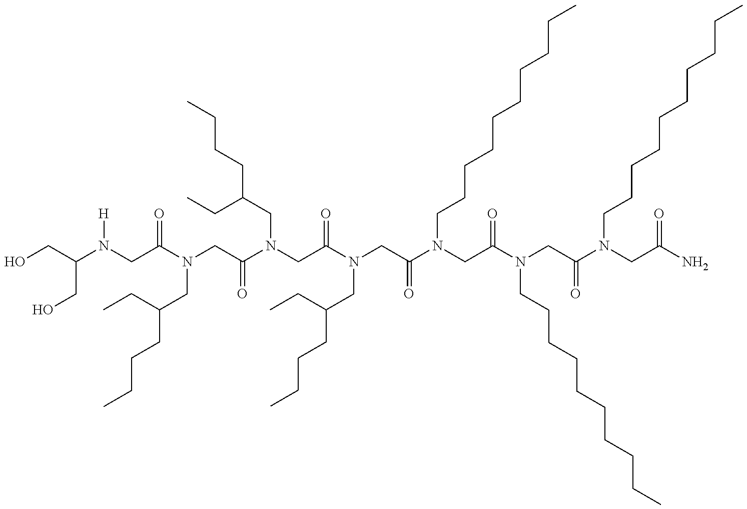 [SMILES: CCCCCCCCCCN(CC(N)=O)C(=O)CN(CCCCCCCCCC)C(=O)CN(CCCCCCCCCC)C(=O)CN(CC(CC)CCCC)C(=O)CN(CC(CC)CCCC)C(=O)CN(CC(CC)CCCC)C(=O)CNC(CO)CO] |
| 56 | 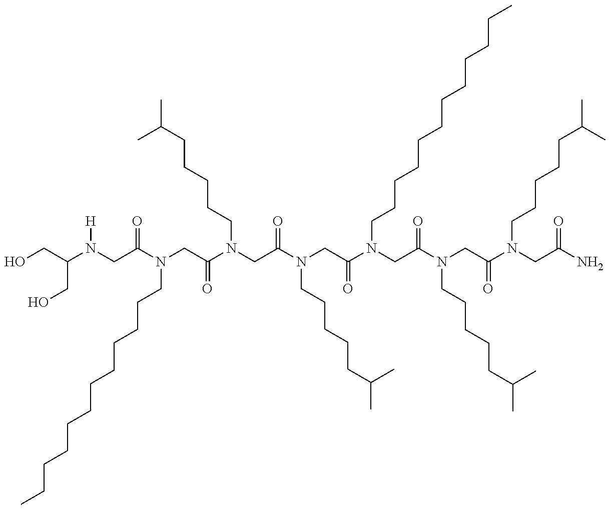 [SMILES: CCCCCCCCCCCCN(CC(=O)N(CCCCCC(C)C)CC(=O)N(CCCCCC(C)C)CC(=O)N(CCCCCCCCCCCC)CC(=O)N(CCCCCC(C)C)CC(=O)N(CCCCCC(C)C)CC(N)=O)C(=O)CNC(CO)CO] |
| 57 | 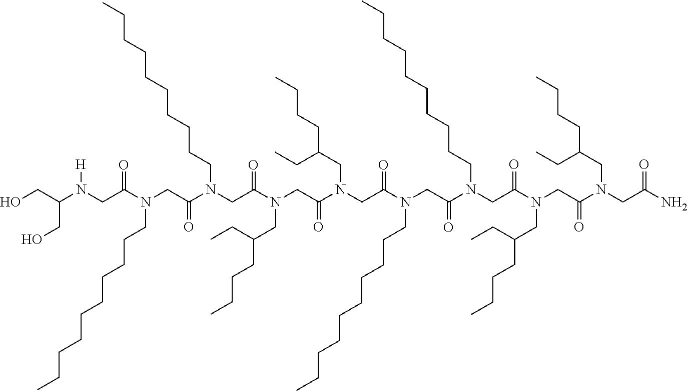 [SMILES: CCCCCCCCCCN(CC(=O)N(CCCCCCCCCC)CC(=O)N(CC(=O)N(CC(=O)N(CCCCCCCCCC)CC(=O)N(CCCCCCCCCC)CC(=O)N(CC(=O)N(CC(N)=O)CC(CC)CCCC)CC(CC)CCCC)CC(CC)CCCC)CC(CC)CCCC)C(=O)CNC(CO)CO] |
| 58 | 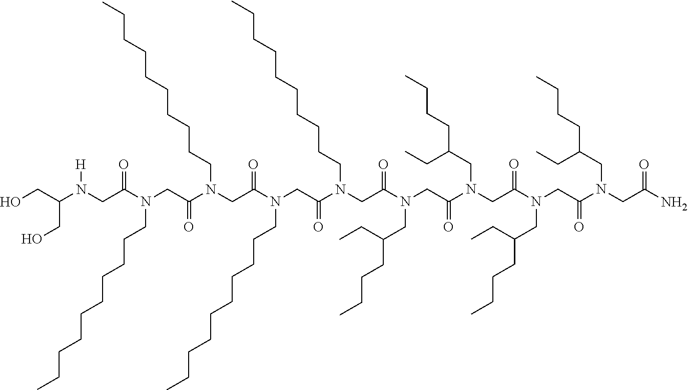 [SMILES: CCCCCCCCCCN(CC(=O)N(CCCCCCCCCC)CC(=O)N(CCCCCCCCCC)CC(=O)N(CCCCCCCCCC)CC(=O)N(CC(=O)N(CC(=O)N(CC(=O)N(CC(N)=O)CC(CC)CCCC)CC(CC)CCCC)CC(CC)CCCC)CC(CC)CCCC)C(=O)CNC(CO)CO] |
| 59 | 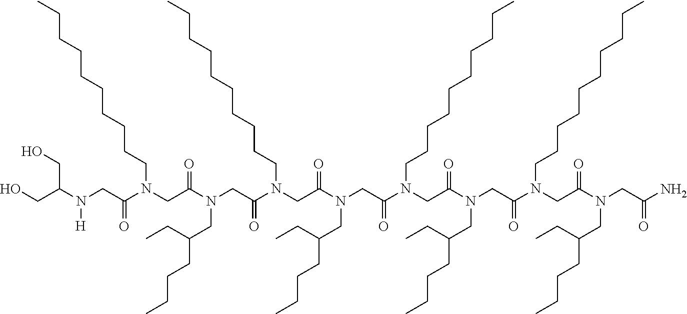 [SMILES: CCCCCCCCCCN(CC(=O)N(CC(=O)N(CCCCCCCCCC)CC(=O)N(CC(=O)N(CCCCCCCCCC)CC(=O)N(CC(=O)N(CCCCCCCCCC)CC(=O)N(CC(N)=O)CC(CC)CCCC)CC(CC)CCCC)CC(CC)CCCC)CC(CC)CCCC)C(=O)CNC(CO)CO] |
| 60 | 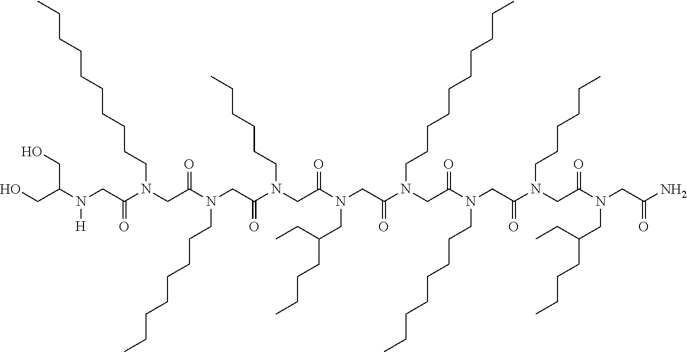 [SMILES: CCCCCCCCCCN(CC(=O)N(CCCCCCCC)CC(=O)N(CCCCCC)CC(=O)N(CC(=O)N(CCCCCCCCCC)CC(=O)N(CCCCCCCC)CC(=O)N(CCCCCC)CC(=O)N(CC(N)=O)CC(CC)CCCC)CC(CC)CCCC)C(=O)CNC(CO)CO] |
| 61 | 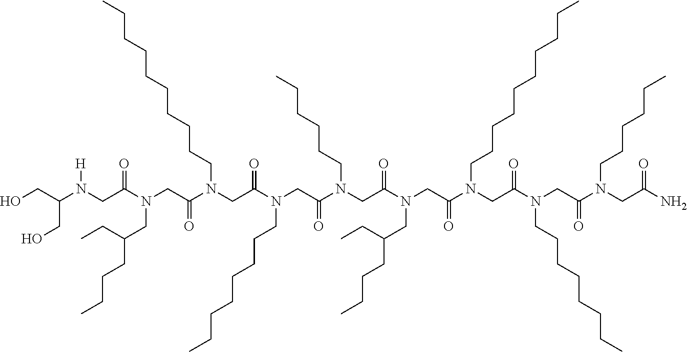 [SMILES: CCCCCCCCCCN(CC(=O)N(CCCCCCCC)CC(=O)N(CCCCCC)CC(=O)N(CC(=O)N(CCCCCCCCCC)CC(=O)N(CCCCCCCC)CC(=O)N(CCCCCC)CC(N)=O)CC(CC)CCCC)C(=O)CN(CC(CC)CCCC)C(=O)CNC(CO)CO] |

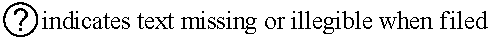

The properties of the peptoids disclosed herein may be advantageously compared to the properties of other peptoids, including the comparative peptoids disclosed in Table C.

TABLE C
Comparative peptoids

| Compound | Structure |
|---|---|
| C1 | 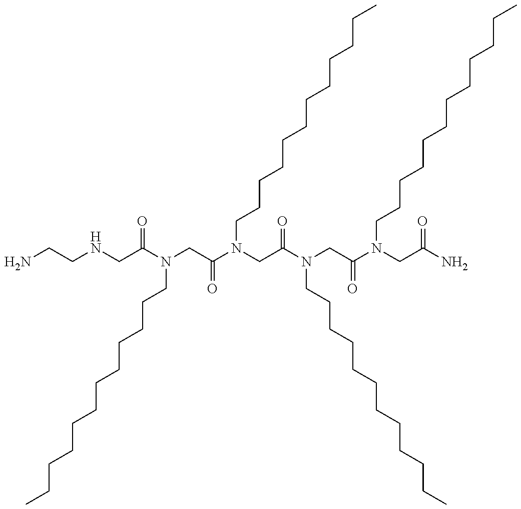 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCN] |
| C2 | 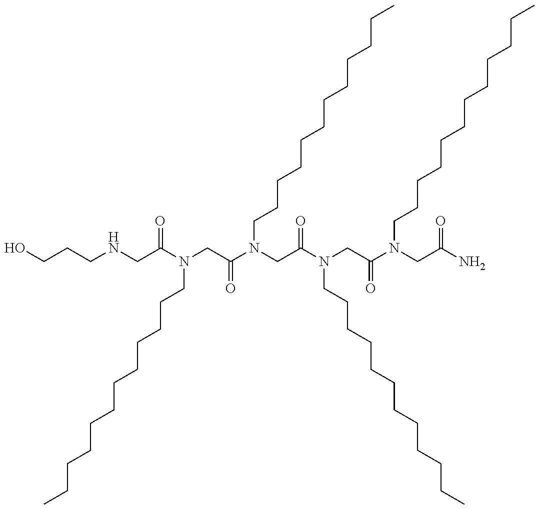 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCO] |
| C3 | 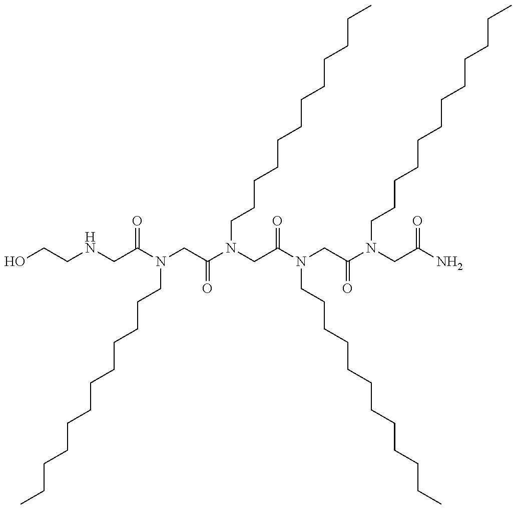 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCO] |
| C4 | 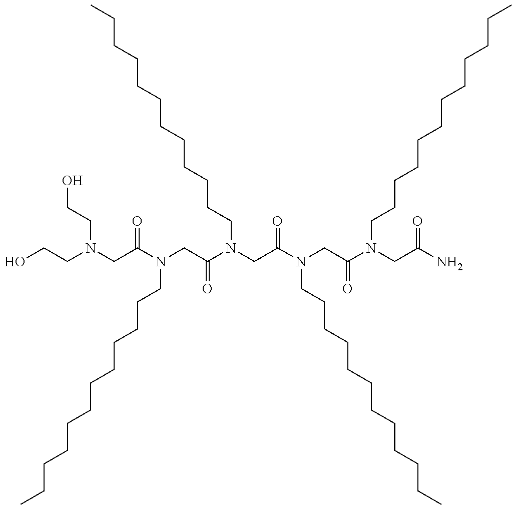 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCO)CCO] |
| C5 | 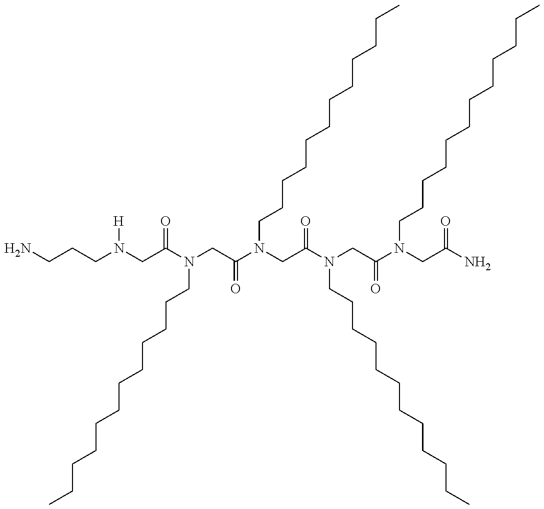 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCN] |
| C6 | 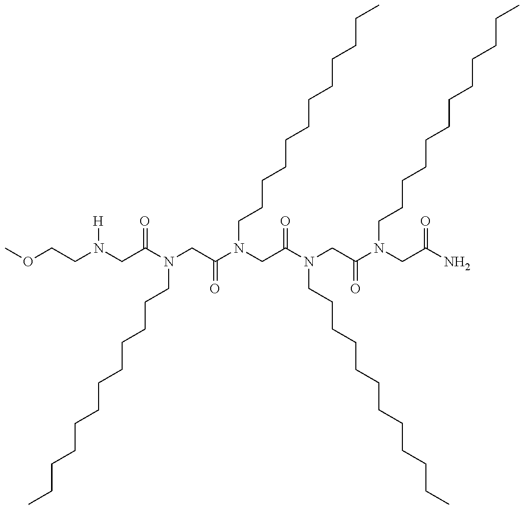 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCOC] |
| C7 | 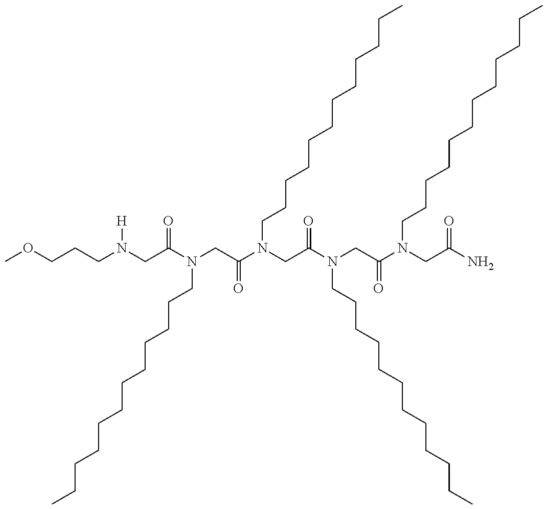 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCOC] |
| C8 | 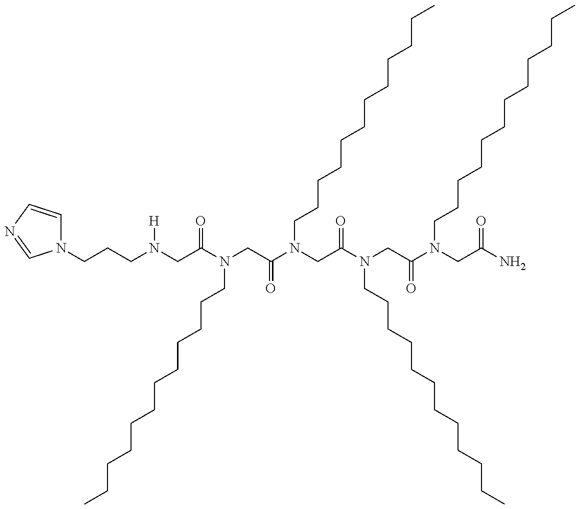 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCn1ccnc1] |
| C9 | 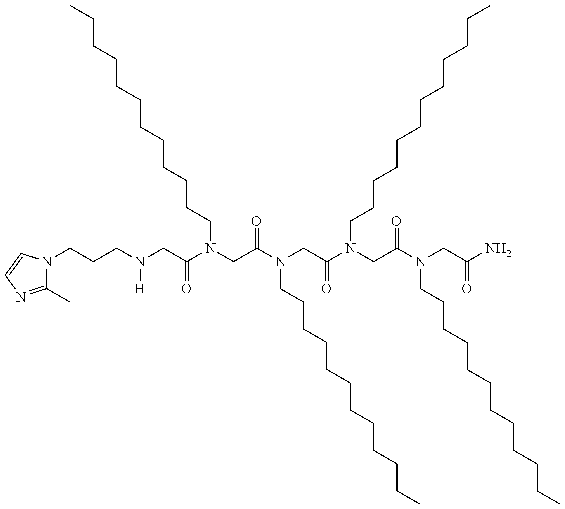 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCn1ccnc1C] |
| C10 | 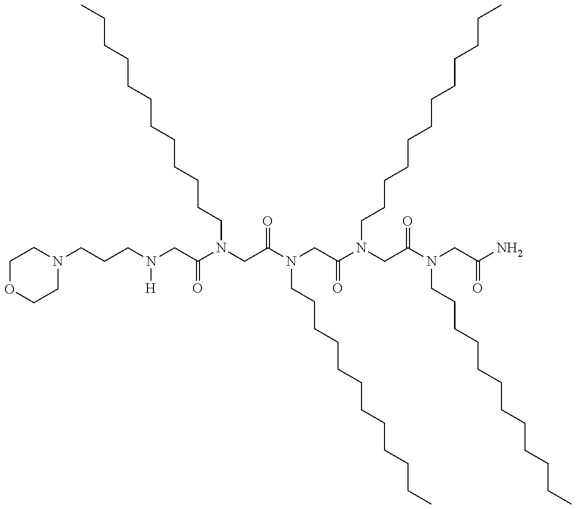 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCN1CCOCC1] |
| C11 | 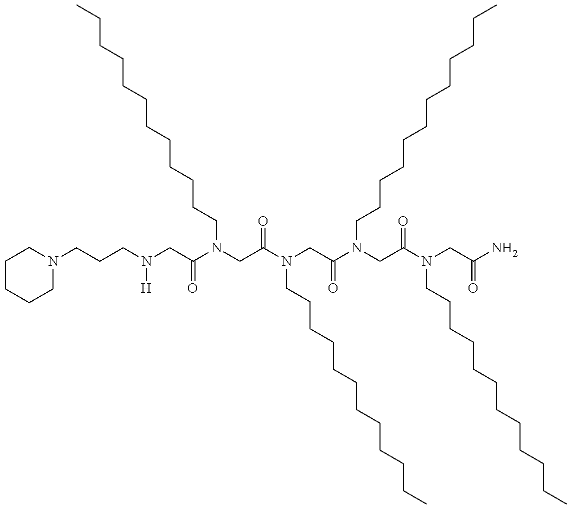 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCCN1CCCCC1] |
| C12 | 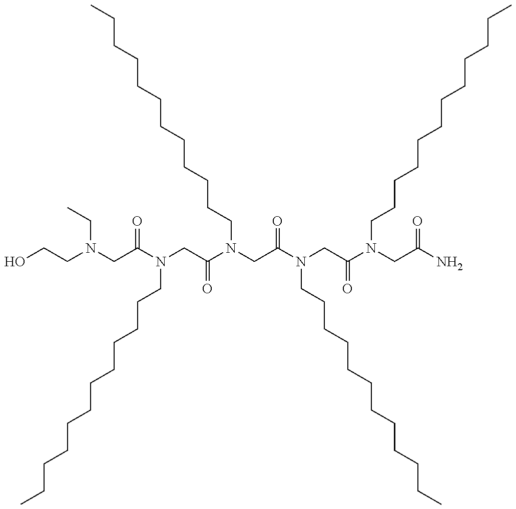 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CC)CCO] |
| C13 | 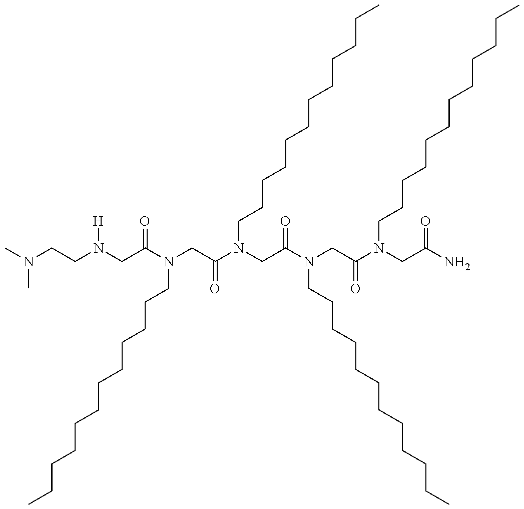 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCN(C)C] |

In some implementations the compound of Formula (I) is compound 1, 6, 21, or 30. In some cases, the compound of Formula (I) is compound 1. In some cases, the compound of Formula (I) is compound 6. In some cases, the compound of Formula (I) is compound 21. In some cases, the compound of Formula (I) is compound 30.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

The compounds described herein can exist in free form, or where appropriate, as a pharmaceutically acceptable salt. As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio. Pharmaceutically acceptable salts are well known in the art. Pharmaceutically acceptable salts of the compounds described herein include those derived from suitable inorganic and organic acids and bases. These salts can be prepared in situ during the final isolation and purification of the compounds. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N⁺(C₁₋₄alkyl)₄ salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This disclosure also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In implementations, the delivery vehicle composition comprises between about 25 mol % to about 70 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. The unit “mol %” or “molar percentage” refers to the number of moles of a particular component of the delivery vehicle composition divided by the total number of moles of all components in the delivery vehicle composition, times 100%. The polyanionic cargo is not calculated as part of the total number of moles of the delivery vehicle composition. In some cases, the delivery vehicle composition comprises between about 30 mol % to about 60 mol %, or about 35 mol % to about 55 mol %, or about 30 mol % to about 45 mol %, or about 35 mol % to about 40 mol %, or about 45 mol % to about 60 mol %, or about 50 mol % to about 55 mol %, or about 38 mol % to about 52 mol %, or about 38 mol %, or about 52 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some implementations, the delivery vehicle composition comprises less than about 50 mol % of the hydroxyalkyl-capped cationic peptoids, such as 2-aminopropane-1,3-diol-capped cationic peptoids, such as less than about 49 mol %, less than about 48 mol %, less than about 47 mol %, less than about 46 mol %, less than about 45 mol %, less than about 44 mol %, less than about 43 mol %, less than about 42 mol %, less than about 41 mol %, less than about 40 mol %, less than about 39 mol %, less than about 38 mol %, less than about 37 mol %, less than about 36 mol %, less than about 35 mol %, less than about 34 mol %, less than about 33 mol %, less than about 32 mol %, less than about 31 mol %, less than about 30 mol %; and greater than about 20 mol % of the hydroxyalkyl-capped cationic peptoids, such as 2-aminopropane-1,3-diol-capped cationic peptoids, such as greater than about 21 mol %, greater than about 22 mol %, greater than about 23 mol %, greater than about 24 mol %, greater than about 25 mol %, greater than about 26 mol %, greater than about 27 mol %, greater than about 28 mol %, greater than about 29 mol %, greater than about 30 mol %, greater than about 31 mol %, greater than about 33 mol %, greater than about 34 mol %, greater than about 35 mol %, greater than about 36 mol %, greater than about 38 mol %, greater than about 39 mol %, greater than about 40 mol %, greater than about 41 mol %, greater than about 42 mol %, greater than about 43 mol %, or greater than about 44 mol % of the hydroxyalkyl-capped cationic peptoids, such as 2-aminopropane-1,3-diol-capped cationic peptoids, based on the total number of moles of components in the delivery vehicle composition. In some implementations, the delivery vehicle composition comprises less than about 50 mol % of the hydroxyalkyl-capped cationic peptoids, such as 2-aminopropane-1,3-diol-capped cationic peptoids, such as less than about 49 mol %, less than about 48 mol %, less than about 47 mol %, less than about 46 mol %, less than about 45 mol %, less than about 44 mol %, less than about 43 mol %, less than about 42 mol %, less than about 41 mol %, less than about 40 mol %, less than about 39 mol %, less than about 38 mol %, less than about 37 mol %, less than about 36 mol %, less than about 35 mol %, less than about 34 mol %, less than about 33 mol %, less than about 32 mol %, less than about 31 mol %, less than about 30 mol %; and greater than about 20 mol % of the hydroxyalkyl-capped cationic peptoids, such as 2-aminopropane-1,3-diol-capped cationic peptoids, such as greater than about 21 mol %, greater than about 22 mol %, greater than about 23 mol %, greater than about 24 mol %, greater than about 25 mol %, greater than about 26 mol %, greater than about 27 mol %, greater than about 28 mol %, greater than about 29 mol %, greater than about 30 mol %, greater than about 31 mol %, greater than about 33 mol %, greater than about 34 mol %, greater than about 35 mol %, greater than about 36 mol %, greater than about 38 mol %, greater than about 39 mol %, or greater than about 40 mol % of the hydroxyalkyl-capped cationic peptoids, such as 2-aminopropane-1,3-diol-capped cationic peptoids, based on the total number of moles of components in the delivery vehicle composition. In some cases, the delivery vehicle composition comprises about 30 mol % to about 49.5 mol %, or about 30 mol % to about 45 mol %, or about 30 mol % to about 35 mol %, or about 40 mol % to about 45 mol %, or about 35 mol % to about 49 mol %, or about 36 mol % to about 48 mol %, or about 38 mol % to about 45 mol %, or about 38 mol % to about 42 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some cases, the delivery vehicle composition comprises about 30 mol % to about 49.5 mol % or about 35 mol % to about 49 mol %, or about 36 mol % to about 48 mol %, or about 38 mol % to about 45 mol %, or about 38 mol % to about 42 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some cases, the delivery vehicle composition comprises about 30 mol % to about 35 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some cases, the delivery vehicle composition comprises about 40 mol % to about 45 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some cases, the delivery vehicle composition comprises about 35 mol % to about 39 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In various cases, the delivery vehicle composition comprises about 39 mol % to about 52 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some implementations, the delivery vehicle composition comprises about 42 mol % to about 49 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In various implementations, the delivery vehicle composition comprises about 50 mol % to about 52 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition. In some cases, the delivery vehicle composition comprises about 30 mol %, about 31 mol %, about 32 mol %, about 33 mol %, about 34 mol %, about 35 mol %, about 36 mol %, about 37 mol %, about 38 mol %, about 39 mol %, about 40 mol %, about 41 mol %, about 42 mol %, about 43 mol %, about 44 mol %, or about 45 mol % of the hydroxyalkyl-capped cationic peptoids, such as 2-aminopropane-1,3-diol-capped cationic peptoids (e.g., a compound of Formula (I), such as compound 1, 6, 21, or 30), based on the total number of moles of components in the delivery vehicle composition.

### Anionic/Zwitterionic Component

In some implementations, the delivery vehicle composition further includes a component that is anionic or zwitterionic (“anionic/zwitterionic component”). The anionic/zwitterionic component can buffer the zeta potential of a particle or a delivery vehicle complex formed from the delivery vehicle composition, without affecting the ratio of the cargo and/or contributing to particle or delivery vehicle endosomal escape through protonation at low pH in the endosome. Zwitterionic components can serve a further function of holding particles together by interacting with both the hydroxyalkyl-capped cationic peptoids, such as 2-aminopropane-1,3-diol-capped cationic peptoids and the polyanionic cargo compounds. Anionic components can also allow for the formation of a core-shell structure of the particle or delivery vehicle, where first a net positive zeta potential particle is made (e.g., by mixing the hydroxyalkyl-capped cationic peptoids, such as 2-aminopropane-1,3-diol-capped cationic peptoids and the cargo at a positive +/− charge ratio), which is then coated with the anionic components. These negatively charged multicomponent system particles would avoid reticuloendothelial system (RES) clearance better than positively charged ones.

Examples of suitable anionic and zwitterionic components of the delivery vehicle composition are described in WO2020/069442 and WO2020/069445, each of which is incorporated herein by reference in its entirety. In some implementations, the zwitterionic component comprises one or more phospholipids. Phospholipids can provide further stabilization to complexes in solution, as well as facilitate cell endocytosis, by virtue of their amphipathic character and ability to disrupt the cell membrane.

In some implementations, the one or more phospholipids are selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In some cases, the phospholipid is DSPC, DOPE, or a combination thereof. In various implementations, the phospholipid is DSPC. In various cases, the phospholipid is DOPE.

In implementations, the delivery vehicle composition comprises between about 1 mol % to about 40 mol % of the phospholipid (e.g., DSPC or DOPE), based on the total number of moles of components in the delivery vehicle composition. In some cases, the delivery vehicle composition comprises between about 3 mol % to about 30 mol %, or about 5 mol % to about 15 mol %, or about 5 mol % to about 10 mol %, or about 10 mol % to about 15 mol %, or about 9 mol % to about 12 mol %, or about 7 mol % to about 11 mol %, or about 7 mol % to about 12 mol %, or about 10 mol % to about 14 mol %, or about 9 mol %, or about 12 mol % of the phospholipid (e.g., DSPC or DOPE), based on the total number of moles of components in the delivery vehicle composition. In some cases, the delivery vehicle composition comprises between about 10 mol % to about 11 mol % of the phospholipid (e.g., DSPC or DOPE), based on the total number of moles of components in the delivery vehicle composition. In some cases, the delivery vehicle composition comprises about 10.0 mol %, about 10.1 mol %, about 10.2 mol %, about 10.3 mol %, about 10.4 mol %, about 10.5 mol %, about 10.6 mol %, about 10.7 mol %, about 10.8 mol %, about 10.9 mol %, or about 11.0 mol % of the phospholipid (e.g., DSPC or DOPE), based on the total number of moles of components in the delivery vehicle composition.

### Neutral Lipid Component

In some implementations, the delivery vehicle composition further includes a component that is a neutral lipid (“neutral lipid component”). The neutral lipid component can be designed to degrade or hydrolyze to facilitate in vivo clearance of the multicomponent delivery system. Contemplated neutral lipid components include, for example, naturally-occurring lipids and lipidated peptoids comprising lipid moieties at the N-position of the peptoid. Further examples of lipidated petoids are described in WO2020/069442 and WO2020/069445, each of which is incorporated herein by reference in its entirety.

In some cases, the neutral lipid component of the delivery vehicle composition comprises one or more sterols. In some implementations, the one more sterols are selected from the group consisting of cholesterol, fecosterol, sitosterol, ergosterol, campesterol, stigmasterol, brassicasterol, tomatidine, ursolic acid, alpha-tocopherol, and mixtures thereof. In some cases, the sterol comprises cholesterol. In implementations, the delivery vehicle composition comprises between about 10 mol % to about 80 mol % of the sterol (e.g., cholesterol), based on the total number of moles of components in the delivery vehicle composition. In some cases, the delivery vehicle composition comprises between about 20 mol % to about 70 mol %, or about 25 mol % to about 60 mol %, or about 30 mol % to about 55 mol %, or about 35 mol % to about 50 mol %, or about 25 mol % to about 45 mol %, or about 40 mol % to about 60 mol %, or about 30 mol % to about 40 mol %, or about 45 mol % to about 55 mol %, or about 35 mol %, or about 50 mol % of the sterol (e.g., cholesterol), based on the total number of moles of components in the delivery vehicle composition. In some cases, the delivery vehicle composition comprises between about 40 mol % to about 55 mol %, or about 40 mol % to about 45 mol %, or about 50 mol % to about 55 mol % of the sterol (e.g., cholesterol), based on the total number of moles of components in the delivery vehicle composition. In some cases, the delivery vehicle composition comprises about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, or about 55 mol % of the sterol (e.g., cholesterol), based on the total number of moles of components in the delivery vehicle composition.

### Shielding Component

In some implementations, the delivery vehicle composition further comprises a shielding component. The shielding component can increase the stability of the particle or delivery vehicle in vivo by serving as a steric barrier, thus improving circulation half-life. Examples of suitable shielding components are described in WO2020/069442 and WO2020/069445, each of which is incorporated herein by reference in its entirety.

In some implementations, the shielding component comprises one or more PEGylated lipids. As used herein, a “PEGylated lipid” includes any lipid or lipid-like compound covalently bound to a polyethylene glycol moiety. Suitable lipid moieties for the PEGylated lipid can include, for example, branched or straight chain aliphatic moieties that can be unsubstituted or substituted, or moieties derived from natural lipid compounds, including fatty acids, sterols, and isoprenoids, that either be unsubstituted or substituted.

In some implementations, the lipid moieties may include branched or straight chain aliphatic moieties having from about 6 to about 50 carbon atoms or from about 10 to about 50 carbon atoms. The aliphatic moieties can comprise, in some implementations, one or more heteroatoms, and/or one or more double or triple bonds (i.e., saturated or mono- or poly-unsaturated). In some cases, the lipid moieties may include aliphatic, straight chain or branched moieties, each hydrophobic tail independently having from about 8 to about 30 carbon atoms or from about 6 to about 30 carbon atoms, wherein the aliphatic moieties can be unsubstituted or substituted. In various implementations, the lipid moieties may include, for example, aliphatic carbon chains derived from fatty acids and fatty alcohols. In some implementations, each lipid moiety is independently C₈-C₂₄-alkyl or C₈-C₂₄-alkenyl, wherein the C₈-C₂₄-alkenyl can be, in some cases, mono- or poly-unsaturated.

Natural lipid moieties employed in the practice of the present disclosure can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic aromatic groups such as optionally substituted aryl or arylalkyl moieties, including for example naphthalenyl or ethylbenzyl, or lipids comprising ester functional groups including, for example, sterol esters and wax esters.

In some implementations, the one or more PEGylated lipids are selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and any combinations thereof. In some implementations, the PEGylated lipids comprise a PEG-modified sterol. In various implementations, the PEGylated lipids comprise PEG-modified cholesterol. In some implementations, the PEGylated lipid is a PEG-modified ceramide. In some cases, the PEG-modified ceramine is selected from the group consisting of N-octanoyl-sphingosine-1-{succinyl[methoxy(polyethylene glycol)]} and N-palmitoyl-sphingosine-1-{succinyl[methoxy(polyethylene glycol)]}, and any combination thereof.

In some implementations, the PEGylated lipids are PEG-modified phospholipids, wherein the phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In various implementations, the phospholipid is DOPE.

In some implementations, the one or more PEGylated lipids comprise a PEG-modified phosphatidylethanol. In some implementations, the PEGylated lipid is a PEG-modified phosphatidylethanol selected from the group consisting of PEG-modified DMPE (DMPE-PEG), PEG-modified DSPE (DSPE-PEG), PEG-modified DPPE (DPPE-PEG), and PEG-modified DOPE (DOPE-PEG).

In various implementations, the PEGylated lipid is selected from the group consisting of dimyristoylglycerol-polyethylene glycol (DMG-PEG), distearoylglycerol-polyethylene glycol (DSG-PEG), dipalmitoylglycerol-polyethylene glycol (DPG-PEG), and dioleoylglycerol-polyethylene glycol (DOG-PEG). In some implementations, the PEG lipid is DMG-PEG.

The molecular weights of the PEG chain in the foregoing PEGylated lipids can be tuned, as desired, to optimize the properties of the delivery vehicle compositions. In some implementations, the PEG chain has a molecular weight between 350 and 6,000 g/mol, between 1,000 and 5,000 g/mol, or between 2,000 and 5,000 g/mol, or between about 1,000 and 3,000 g/mol, or between abut 1,500 and 4,000 g/mol. In some cases, the PEG chain of the PEG lipid has a molecular weight of about 350 g/mol, 500 g/mol, 600 g/mol, 750 g/mol, 1,000 g/mol, 2,000 g/mol, 3,000 g/mol, 5,000 g/mol, or 10,000 g/mol. In some implementations, the PEG chain of the PEGylated lipid has a molecular weight of about 500 g/mol, 750 g/mol, 1,000 g/mol, 2,000 g/mol or 5,000 g/mol. The PEG chain can be branched or linear. In some cases, the PEGylated lipid is dimyristoylglycerol-polyethylene glycol 2000 (DMG-PEG 2000).

In implementations, the delivery vehicle composition comprises between about 1 mol % to about 5 mol % of the PEGylated lipid (e.g., DMG-PEG 2000), based on the total number of moles of components in the delivery vehicle composition. In some cases, the delivery vehicle composition comprises between about 1 mol % to about 3 mol %, or about 1 mol % to about 2 mol %, or about 2 mol % to about 5 mol %, or about 0.5 mol % to about 1.5 mol %, or about 1.5 mol % to about 2.5 mol %, or about 1.5 mol % to about 2.0 mol %, or about 2.0 mol % to about 2.5 mol %, or about 1 mol %, or about 1.5 mol %, or about 2 mol %, or about 2.5 mol %, or about 3 mol %, or about 3.5 mol %, or about 4 mol %, or about 5 mol % of the PEGylated lipid (e.g., DMG-PEG 2000), based on the total number of moles of components in the delivery vehicle composition. In some cases, the delivery vehicle composition comprises between about 1 mol % to about 3 mol %, or about 1 mol % to about 2 mol %, or about 2 mol % to about 5 mol %, or about 0.5 mol % to about 1.5 mol %, or about 1.5 mol % to about 2.5 mol %, or about 1 mol %, or about 1.5 mol %, or about 2 mol %, or about 2.5 mol %, or about 3 mol %, or about 3.5 mol %, or about 4 mol %, or about 5 mol % of the PEGylated lipid (e.g., DMG-PEG 2000), based on the total number of moles of components in the delivery vehicle composition. In some cases, the delivery vehicle composition comprises about 1.5 mol %, 1.6 mol %, 1.7 mol %, 1.8 mol %, 1.9 mol %, 2.0 mol %, 2.1 mol %, 2.2 mol %, 2.3 mol %, 2.4 mol %, or about 2.5 mol % of the PEGylated lipid (e.g., DMG-PEG 2000), based on the total number of moles of components in the delivery vehicle composition.

### Representative Examples

Non-limiting delivery vehicle combinations are described below. As previously described, the unit “mol %” or “molar percentage” refers to the number of moles of a particular component of the delivery vehicle composition divided by the total number of moles of all components in the delivery vehicle composition, times 100%.

In some implementations, the delivery vehicle composition comprises at least 99 mol % the cationic component and less than about 1 mol % shielding component (e.g., Formula F1A in Table 2). In some cases, the delivery vehicle composition comprises less than about 20 mol % of the cationic component, less than about 5 mol % of a shielding component, and more than about 75 mol % of a mixture of the anionic/zwitterionic component and the neutral lipid component (e.g., Formula F2A and Formula F4A in Table 2). In some cases, the delivery vehicle composition comprises about 30 to about 45 mol % of the cationic component, about 50 to about 70 mol % of a mixture of the anionic/zwitterionic component and the neutral lipid component, and about 1.5 to about 4.5 mol % of the shielding component (e.g., Formula F3A and Formula F5A in Table 2). In various cases, the delivery vehicle composition comprises about 15 to about 35 mol % of the cationic component, about 60 to about 80 mol % of a mixture of the anionic/zwitterionic component and the neutral lipid component, and about 1.5 to about 3.0 mol % of a shielding component (e.g., Formula F2A and Formula F3A in Table 2). In some implementations, the delivery vehicle composition comprises about 15 to about 35 mol % of the cationic component, about 10-about 20 mol % of an anionic/zwitterionic component, about 50 to about 65 mol % of a neutral lipid component, and about 1.5 to about 3.0 mol % of a shielding component (e.g., Formula F2A and Formula F3A in Table 2). In various implementations, the delivery vehicle composition comprises about 10 to about 20 mol % of the cationic component, about 75 to about 89 mol % of a lipid component, and about 1 to about 5 mol % of a shielding component (e.g., Formula F4A in Table 2). In some cases, the delivery vehicle composition comprises about 40 to about 50 mol % of the cationic component, about 50 to about 59 mol % of an anionic/zwitterionic component, and about 1 to about 5 mol % shielding component (e.g., Formula F5A in Table 2). In various cases, the delivery vehicle composition comprises about 30 to about 50 mol % of the cationic component, about 50 to about 70 mol % of a neutral lipid component, and about 1 to about 5 mol % shielding component (e.g., Formula F6A in Table 2). In various cases, the delivery vehicle composition comprises about 40 to about 45 mol % of the cationic component, about 50 to about 60 mol % of a mixture of the anionic/zwitterionic component and the neutral lipid component, and about 1.5 to about 2.0 mol % of a shielding component (e.g., Formula F6.1 and Formula F6.2 in Table 2). In some implementations, the delivery vehicle composition comprises about 40 to about 45 mol % of the cationic component, about 10 to about 15 mol % of an anionic/zwitterionic component, about 40 to about 45 mol % of a neutral lipid component, and about 1.5 to about 2.0 mol % of a shielding component (e.g., Formula F6.1 and Formula F6.2 in Table 2). In various cases, the delivery vehicle composition comprises about 30 to about 35 mol % of the cationic component, about 60 to about 70 mol % of a mixture of the anionic/zwitterionic component and the neutral lipid component, and about 2.0 to about 3.0 mol % of a shielding component (e.g., Formula F6.3 in Table 2). In some implementations, the delivery vehicle composition comprises about 30 to about 35 mol % of the cationic component, about 10 to about 15 mol % of an anionic/zwitterionic component, about 50 to about 55 mol % of a neutral lipid component, and about 2.0 to about 3.0 mol % of a shielding component (e.g., Formula F6.3 in Table 2). The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG2000.

In some implementations, the delivery vehicle composition comprises about 30 mol % to about 60 mol % (e.g., about 35 mol % to about 39 mol %, or about 39 mol % to about 52 mol %, or about 42 mol % to about 49 mol %, or about 50 mol % to about 52 mol %) of the cationic component; about 3 mol % to about 20 mol % of the anionic/zwitterionic component, about 25 mol % to about 60 mol % of the neutral lipid compound, and about 1 mol % to about 5 mol % of the shielding component. In various implementations, the delivery vehicle composition comprises about 35 to about 55 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 38 to about 52 mol % of the cationic component; about 9-about 12 mol % of the anionic/zwitterionic component, about 35 mol % to about 50 mol % of the neutral lipid compound, and about 1 mol % to about 2 mol % of the shielding component. In some cases, the delivery vehicle composition comprises about 30 mol % to about 49 mol % of the compound of Formula (I); about 5 mol % to about 15 mol % of the phospholipid, about 30 mol % to about 55 mol % of the sterol, and about 1 mol % to about 3 mol % of the PEGylated lipid. In some cases, the composition comprises about 35 mol % to about 49 mol % of the compound or salt of Formula (I); about 7 mol % to about 12 mol % of the phospholipid, about 35 mol % to about 50 mol % of the sterol, and about 1 mol % to about 2 mol % of the PEGylated lipid. The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG2000.

In some implementations, the delivery vehicle composition comprises about 30 mol % to about 45 mol % of the cationic component; about 5 mol % to about 15 mol % of the anionic/zwitterionic component, about 40 mol % to about 60 mol % of the neutral lipid compound, and about 1 mol % to about 5 mol % of the shielding component. In various implementations, the delivery vehicle composition comprises about 35 mol % to about 40 mol % of the cationic component; about 8 mol % to about 12 mol % of the anionic/zwitterionic component, about 45 mol % to about 50 mol % of the neutral lipid compound, and about 1 mol % to about 3 mol % of the shielding component. In various implementations, the delivery vehicle composition comprises about 38.2 mol % of the cationic component; about 11.8 mol % of the anionic/zwitterionic component, about 48.2 mol % of the neutral lipid compound, and about 1.9 mol % of the shielding component (“Form F2”). The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG-2000. In some implementations, the delivery vehicle composition comprises Form F2, as shown in Table 2, below. In some implementations, the delivery vehicle composition comprises about 38.2 mol % of compound 1, 6, 21, or 30, about 11.8 mol % of DSPC, about 48.2 mol % of cholesterol, and about 1.9 mol % of DMG-PEG-2000.

In some implementations, the delivery vehicle composition comprises about 45 to about 55 mol % of the cationic component; about 5 mol % to about 15 mol % of the anionic/zwitterionic component, about 35 mol % to about 55 mol % of the neutral lipid compound, and about 1 mol % to about 5 mol % of the shielding component. In various implementations, the delivery vehicle composition comprises about 48 mol % to about 52 mol % of the cationic component; about 5 mol % to about 12 mol % of the anionic/zwitterionic component, about 38 mol % to about 42 mol % of the neutral lipid compound, and about 1 mol % to about 3 mol % of the shielding component. In various implementations, the delivery vehicle composition comprises about 51.3 mol % of the cationic component; about 9.3 mol % of the anionic/zwitterionic component, about 38.0 mol % of the neutral lipid compound, and about 1.5 mol % of the shielding component (“Form F6/17”). The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises Form F6/17, as shown in Table 2, below. In some implementations, the delivery vehicle composition comprises about 51.3 mol % of compound 1, 6, 21, or 30, about 9.3 mol % of DSPC, about 38.0 mol % of cholesterol, and about 1.5 mol % of DMG-PEG 2000.

In some implementations, the delivery vehicle composition comprises about 30 mol % to about 49 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 48 mol % to about 52 mol % of the cationic component; about 5 mol % to about 12 mol % of the anionic/zwitterionic component, about 38 mol % to about 42 mol % of the neutral lipid compound, and about 1 mol % to about 3 mol % of the shielding component. In various implementations, the delivery vehicle composition comprises about 42.6 mol % of the cationic component; about 10.0 mol % of the anionic/zwitterionic component, about 44.7 mol % of the neutral lipid compound, and about 1.7 mol % of the shielding component. The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises Form F6/12 or Form F6/15, as shown in Table 2, below. In some implementations, the delivery vehicle composition comprises about 42.6 mol % of compound 1, 6, 21, or 30, about 10.9 mol % of DSPC, about 44.7 mol % of cholesterol, and about 1.7 mol % of DMG-PEG 2000. In some implementations, the delivery vehicle composition comprises about 48.1 mol % of compound 1, 6, 21, or 30, about 9.9 mol % of DSPC, about 40.4 mol % of cholesterol, and about 1.6 mol % of DMG-PEG 2000.

In some implementations, the delivery vehicle composition comprises about 40 mol % to about 49 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 42 mol % to about 46 mol % of the cationic component; about 7 mol % to about 12 mol % of the anionic/zwitterionic component, about 41 mol % to about 45 mol % of the neutral lipid compound, and about 1 mol % to about 2 mol % of the shielding component. In various implementations, the delivery vehicle composition comprises about 44.4 mol % of the cationic component; about 10.6 mol % of the anionic/zwitterionic component, about 43.3 mol % of the neutral lipid compound, and about 1.7 mol % of the shielding component. In various implementations, the delivery vehicle composition comprises about 44.4 mol % of the cationic component; about 10.6 mol % of the anionic/zwitterionic component, about 43.4 mol % of the neutral lipid compound, and about 1.7 mol % of the shielding component. The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises F6.1 or F6.2, as shown in Table 2, below. In some implementations, the delivery vehicle composition comprises about 44.4 mol % of compound 1, 6, 21, or 30, about 10.6 mol % of DSPC, about 43.3 mol % of cholesterol, and about 1.7 mol % of DMG-PEG 2000. In some implementations, the delivery vehicle composition comprises about 44.4 mol % of compound 1, 6, 21, or 30, about 10.6 mol % of DSPC, about 43.4 mol % of cholesterol, and about 1.7 mol % of DMG-PEG 2000.

In some implementations, the delivery vehicle composition comprises about 30 mol % to about 39 mol % of the cationic component; about 5 mol % to about 15 mol % of the anionic/zwitterionic component, about 30 mol % to about 55 mol % of the neutral lipid compound, and about 1 mol % to about 3 mol % of the shielding component. In various implementations, the delivery vehicle composition comprises about 30 mol % to about 35 mol % of the cationic component; about 7 mol % to about 12 mol % of the anionic/zwitterionic component, about 50 mol % to about 55 mol % of the neutral lipid compound, and about 2 mol % to about 3 mol % of the shielding component. In various implementations, the delivery vehicle composition comprises about 33.1 mol % of the cationic component; about 10.5 mol % of the anionic/zwitterionic component, about 53.8 mol % of the neutral lipid compound, and about 2.5 mol % of the shielding component. The cationic component can be any cationic component described herein, such as any of the compounds of Formula (I) (e.g., the compounds listed in Table 1, such as compound 1, 6, 21, or 30). In some implementations, the cationic compound is compound 1, 6, 21, or 30. The anionic/zwitterionic component can be any anionic/zwitterionic component described herein (e.g., a phospholipid). In some implementations, the anionic/zwitterionic component is DSPC or DOPE. The neutral lipid component can be any neutral lipid described herein (e.g., a sterol). In some implementations, the neutral lipid component is cholesterol. The shielding component can be any shielding component described herein (e.g., PEGylated lipids). In some implementations, the shielding component is DMG-PEG 2000. In some implementations, the delivery vehicle composition comprises F6.3, as shown in Table 2, below. In some implementations, the delivery vehicle composition comprises about 33.1 mol % of compound 1, 6, 21, or 30, about 10.5 mol % of DSPC, about 53.8 mol % of cholesterol, and about 2.5 mol % of DMG-PEG 2000.

Non-limiting examples delivery vehicle compositions of the disclosure based on compounds of Formula (I) (e.g., compound 1, 6, 21, or 30) as the cationic component (characterized by mol %) can be found in Table 2, below.

TABLE 2
Delivery Vehicle Compositions

| Mole | | Anionic or | Non-cationic | |
|---|---|---|---|---|
| Percentages | Cationic | Zwitterionic | lipid | Shielding |
| (mol %) | component | component | component | component |
| F1A | 99.1 | 0 | 0 | 0.9 |
| F2A | 17.9 | 16.4 | 62.9 | 2.8 |
| F3A | 32.9 | 13.4 | 51.7 | 2.0 |
| F4A | 17.1 | 0 | 80.2 | 2.7 |
| F5A | 42.3 | 53.3 | 0 | 4.4 |
| F6A | 38.0 | 0 | 59.7 | 2.3 |
| F1 | 21.4 | 15.7 | 60.3 | 2.7 |
| F2 | 38.2 | 11.8 | 48.2 | 1.9 |
| F3 | 36.0 | 10.0 | 51.7 | 2.4 |
| F4 | 30.2 | 29.6 | 39.5 | 0.7 |
| F5 | 32.0 | 16.7 | 48.7 | 2.6 |
| F6/12 | 42.6 | 10.9 | 44.7 | 1.7 |
| F6/15 | 48.1 | 9.9 | 40.4 | 1.6 |
| F6/17 | 51.3 | 9.3 | 38.0 | 1.5 |
| F6.1 | 44.4 | 10.6 | 43.3 | 1.7 |
| F6.2 | 44.4 | 10.6 | 43.4 | 1.7 |
| F6.3 | 33.1 | 10.5 | 53.8 | 2.5 |

In some cases, the delivery vehicle composition is F6.1, F6.2, or F6.3. In some cases, the delivery vehicle composition is F1A, F2A, F3A, F4A, F5A, F6A, F1, F2, F3, F4, F5, F6/12, F6/15, or F6/17. In some cases, the delivery vehicle composition is D1, D2, D3, D4, D5, D6, D7, D8, D9, D10, D11, D12, D13, D14, D15, D16, D17, D18, D19, D20, D21, D22, D23, D24, D25, D26, D27, D28, D29, D30, D31, or D32.

### Delivery Vehicle Complexes (DV)

The delivery vehicle compositions disclosed herein can form complexes with one or more polyanionic compounds (e.g., nucleic acids) through an electrostatic interaction between the cationic component of the delivery vehicle composition and the polyanionic compound. Thus, a delivery vehicle complex refers to a mixture comprising a delivery vehicle composition, as disclosed herein, and a polyanionic compound. The complexes, in some instances, permit a high amount of cargo encapsulation, are stable, and demonstrate excellent efficiency and tolerability in vivo. The delivery vehicle complexes, therefore, are useful as delivery vehicles for the transportation of the polyanionic cargo encapsulated therein to a target cell. Additionally or alternatively, the delivery vehicle complexes can include a non-anionic cargo. Accordingly, another aspect of the disclosure relates to a delivery vehicle complex comprising: (1) a delivery vehicle composition, as previously described herein, and (2) a polyanionic compound (or cargo). In some implementations, the delivery vehicle composition complexes with one polyanionic compound (e.g., one RNA). In various implementations, the delivery vehicle composition complexes with two different polyanionic compound (e.g., two different RNAs or an RNA and a DNA). In some implementations, the delivery vehicle composition complexes with three or more different polyanionic compounds (e.g., 3, 4, or 5 different RNAs). In some cases, the multicomponent delivery vehicle system complexes with one or more of a nucleic acid selected from DNA and RNA (e.g., an antigenic RNA and adjuvanting DNA, such as CpG).

The delivery vehicle complexes described herein may be characterized by the relative mass ratio of one of the components of the delivery vehicle composition to the cargo (e.g., a polyanionic compound) in the complex. Mass ratios of the components in the delivery vehicle complex can be readily calculated based upon the known concentrations and volumes of stock solutions of each component used in preparing the complex. Moreover, if non-anionic cargoes are present in the delivery vehicle complex, mass ratios may provide a more accurate representation of the relative amounts of delivery vehicle components to the overall cargo than cation:anion charge ratios, which do not account for non-anionic material. Specifically, the mass ratio of a component refers to the ratio of the mass of this particular component in the system to the mass of the “cargo” in the system. “Cargo” may refer to the total polyanionic compound(s) present in the system. In one example, the polyanionic compound(s) may refer to nucleic acid(s). In one example, the polyanionic compound(s) refer to mRNA(s) encoding at least one protein.

In some implementations, the cationic component and the polyanionic compound of the delivery vehicle complex have a mass ratio between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In some implementations, the cationic component and the polyanionic compound of the delivery vehicle complex have a mass ratio between about 2:1 and about 5:1. In still yet other implementations, the cationic component and the polyanionic compound of the delivery vehicle complex have a mass ratio of about 3:1. In other implementations, the cationic component and the polyanionic compound of the delivery vehicle complex have a mass ratio of about 19:1. In other implementations, the cationic component and the polyanionic compound of the delivery vehicle complex have a mass ratio of about 20:1. In other implementations, the cationic component and the polyanionic compound of the delivery vehicle complex have a mass ratio of about 13:1. In other implementations, the cationic component and the polyanionic compound of the delivery vehicle complex have a mass ratio of about 10:1. In some implementations, the cationic component can be a compound of Formula (I), such as a compound listed in Table 1 (e.g., compound 1, 6, 21, or 30).

In certain implementations wherein the delivery vehicle complex comprises a nucleic acid as the polyanionic compound, or cargo, the mass ratio of the cationic component and the nucleic acid is between about 0.5:1 and about 20:1, or between about 0.5:1 and about 10:1, or between about 0.5:1 and about 5:1, or between about 1:1 and about 20:1, or between about 1:1 and about 10:1, or between about 1:1 and about 5:1, or between about 2:1 and about 20:1, or between about 2:1 and about 10:1, or between about 2:1 and about 5:1. In certain implementations, the mass ratio of the cationic component and the nucleic acid is between about 2:1 and about 5:1. In still yet other implementations, the mass ratio of the cationic component and the nucleic acid is about 3:1. In other implementations, the mass ratio of the cationic component and the nucleic acid is about 19:1. In other implementations, the mass ratio of the cationic component and the nucleic acid is about 20:1. In other implementations, the mass ratio of the cationic component and the nucleic acid is about 13:1. In other implementations, the mass ratio of the cationic component and the nucleic acid is about 10:1. In some implementations, the cationic component can be a compound of Formula (I), such as a compound listed in Table 1 (e.g., compound 1, 6, 21, or 30).

In some implementations, the mass ratio of the cationic component and the nucleic acid is between about 5:1 to about 25:1, or about 7:1 to about 20:1, or about 10:1 to about 17:1, or about 9.5:1 to about 10.5:1, or about 11:1 to about 17:1. In various implementations, the mass ratio of the cationic component and the nucleic acid is about 20:1. In various implementations, the mass ratio of the cationic component and the nucleic acid is about 19:1. In some implementations, the mass ratio of the cationic component and the nucleic acid is about 17:1. In various implementations, the mass ratio of the cationic component and the nucleic acid is about 15:1. In various implementations, the mass ratio of the cationic component and the nucleic acid is about 13:1. In various implementations, the mass ratio of the cationic component and the nucleic acid is about 12:1. In various implementations, the mass ratio of the cationic component and the nucleic acid is about 10:1. In some implementations, the cationic component can be a compound of Formula (I), as previously described herein, such as a compound listed in Table 1. In various implementations, the cationic component is compound 1, 6, 21, or 30. In some implementations, the polyanionic cargo is a nucleic acid, such as RNA.

In some cases, the mass ratio of the anionic/zwitterionic component and the polyanionic compound is about 2:1 to about 10:1, or about 2:1 to about 3:1, or about 2:1 to about 4:1, or about 5:1 to about 10:1. In some cases, the mass ratio of the anionic/zwitterionic component and the polyanionic compound is about 2:1 to about 10:1, or about 2:1 to about 3:1, or about 5:1 to about 10:1. In some implementations, the mass ratio of the anionic/zwitterionic component and the polyanionic compound is about 4:1. In some implementations, the mass ratio of the anionic/zwitterionic component and the polyanionic compound is about 2.7:1. In some implementations, the anionic/zwitterionic component can be a phospholipid, as previously described herein. In various implementations, the anionic/zwitterionic component is DOPE, DSPC, or a combination thereof. In some cases, the anionic/zwitterionic component is DSPC. In some implementations, the polyanionic cargo is a nucleic acid, such as RNA.

In some cases, the mass ratio of the neutral lipid component and the polyanionic compound is between about 5:1 to about 8:1, or about 4:1 to about 7:1, or about 5:1 to about 6:1, or about 1:1 to about 5:1. In some cases, the mass ratio of the neutral lipid component and the polyanionic compound is between about 4:1 to about 7:1, or about 5:1 to about 6:1, or about 1:1 to about 5:1. In some implementations, the mass ratio of the neutral lipid component and the polyanionic compound is about 5.4:1. In some implementations, the mass ratio of the neutral lipid component and the polyanionic compound is about 8.1:1. In some implementations, the mass ratio of the neutral lipid component and the polyanionic compound is about 6.7:1. In some implementations, the neutral lipid component can be a sterol, as previously described herein. In various implementations, the neutral lipid component is cholesterol. In some implementations, the polyanionic cargo is a nucleic acid, such as RNA.

In some cases, the mass ratio of the shielding component and the polyanionic compound is between about 0.5:1 to about 2.5:1, or about 1:1 to about 2:1, or about 2:1 to about 3:1. In some implementations, the mass ratio of the neutral lipid component and the polyanionic compound is about 2.1:1. In some implementations, the mass ratio of the neutral lipid component and the polyanionic compound is about 1.4:1. In some implementations, the shielding component can be a PEGylated lipid, as previously described herein. In various implementations, the shielding component is DMG-PEG 2000. In some implementations, the polyanionic cargo is a nucleic acid, such as RNA.

In some implementations, the delivery vehicle complex comprises the cationic component and the polyanionic cargo at a mass ratio of about 10:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F2”). In some implementations, the cationic component is a compound of Formula (I), the anionic/zwitterionic component is a phospholipid, the neutral lipid component is cholesterol, and the shielding component is a PEGylated lipid. In some implementations, the polyanionic compound is a nucleic acid, such as RNA. In various implementations, the delivery vehicle complex comprises compound 1, 6, 21, or 30 at a mass ratio of about 10:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid.

In some implementations, the delivery vehicle complex comprises the cationic component and the polyanionic cargo at a mass ratio of about 17:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F6/17”). In some implementations, the cationic component is a compound of Formula (I), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises compound 1, 6, 21, or 30 at a mass ratio of about 17:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid.

In some implementations, the delivery vehicle complex comprises the cationic component and the polyanionic cargo at a mass ratio of about 12:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F6/12”). In some implementations, the cationic component is a compound of Formula (I), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises compound 1, 6, 21, or 30 at a mass ratio of about 12:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid.

In some implementations, the delivery vehicle complex comprises the cationic component and the polyanionic cargo at a mass ratio of about 15:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“Form F6/15”). In some implementations, the cationic component is a compound of Formula (I), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises compound 1, 6, 21, or 30 at a mass ratio of about 15:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid.

In some implementations, the delivery vehicle complex comprises the cationic component and the polyanionic cargo at a mass ratio of about 13:1, the anionic/zwitterionic component and the polyanionic cargo at a mass ratio of about 2.7:1, the neutral lipid component and the polyanionic cargo at a mass ratio of about 5.4:1, and the shielding component and the polyanionic cargo at a mass ratio of about 1.4:1 (“F6.1”). In some implementations, the cationic component is a compound of Formula (I), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises compound 1, 6, 21, or 30 at a mass ratio of about 13:1 with the nucleic acid, DSPC at a mass ratio of about 2.7:1 with the nucleic acid, cholesterol at a mass ratio of about 5.4:1 with the nucleic acid, and DMG-PEG 2000 at a mass ratio of about 1.4 with the nucleic acid.

In some implementations, the delivery vehicle complex comprises the cationic component and the polyanionic cargo at a mass ratio of about 19:1, the anionic/zwitterionic component and the polyanionic cargo at a mass ratio of about 4.0:1, the neutral lipid component and the polyanionic cargo at a mass ratio of about 8.1:1, and the shielding component and the polyanionic cargo at a mass ratio of about 2.1:1 (“F6.2”). In some implementations, the cationic component is a compound of Formula (I), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises compound 1, 6, 21, or 30 at a mass ratio of about 19:1 with the nucleic acid, DSPC at a mass ratio of about 4.0:1 with the nucleic acid, cholesterol at a mass ratio of about 8.1:1 with the nucleic acid, and DMG-PEG 2000 at a mass ratio of about 2.1 with the nucleic acid.

In some implementations, the delivery vehicle complex comprises the cationic component and the polyanionic cargo at a mass ratio of about 9.7, the anionic/zwitterionic component and the polyanionic cargo at a mass ratio of about 2.7:1, the neutral lipid component and the polyanionic cargo at a mass ratio of about 6.7:1, and the shielding component and the polyanionic cargo at a mass ratio of about 2.1:1 (“F6.3”). In some implementations, the cationic component is a compound of Formula (I), the anionic/zwitterionic component is a phospholipid, the neutral lipid component is cholesterol, and the shielding component is a PEGylated lipid. In some implementations, the polyanionic cargo is a nucleic acid, such as RNA. In various implementations, the delivery vehicle complex comprises compound 1, 6, 21, or 30 at a mass ratio of about 9.7:1 with the nucleic acid, DSPC at a mass ratio of about 2.7:1 with the nucleic acid, cholesterol at a mass ratio of about 6.7:1 with the nucleic acid, and DMG-PEG 2000 at a mass ratio of about 2.1 with the nucleic acid.

In still other implementations, the amount of polyanionic cargo present in the delivery vehicle complexes may be characterized by a mass ratio of delivery vehicle composition (e.g., hydroxyalkyl-capped cationic peptoids, such as 2-aminopropane-1,3-diol-capped cationic peptoids, phospholipid, cholesterol, and/or the shielding component in total) to the one or more polyanionic cargo compounds. In some implementations, the mass ratio of the delivery vehicle composition to the one or more polyanionic cargo compounds is between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1. In certain implementations, the mass ratio of the delivery vehicle composition to the one or more polyanionic cargo compounds is between about 5:1 and about 8:1 or between about 6:1 and about 7:1.

In some implementations, the compound of Formula (I) or salt thereof, phospholipid, sterol, and PEGylated lipid are present in a mass ratio of about 20:1.8:7.2:1.8 (“D22”). In some cases, the phospholipid is DSPC. In some cases, the sterol is cholesterol. In some cases, the PEGylated lipid is DMG-PEG2000. In some cases, the compound of Formula (I) can be compound 12, 34, 35, or 41.

In some implementations, the delivery vehicle complexes described herein may be characterized by the ratio of the number cationic groups on the cationic component of the delivery vehicle composition to the number of anionic phosphate groups on the nucleic acid cargo. In some implementations, the delivery vehicle complex comprises the cationic component and the nucleic acid at a cation:anion charge ratio of between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1, or between about 3:1 and about 8:1, or between about 3:1 and about 7:1, or between about 3:1 and about 4:1, or between about 4:1 and about 5:1, or between about 6:1 and about 7:1, or between about 7:1 and about 8:1. In some implementations, the delivery vehicle complex comprises the cationic component and the nucleic acid at a cation:anion charge ratio of between about 0.5:1 and about 20:1, between about 0.5:1 and about 10:1, between about 0.5:1 and about 5:1, between about 1:1 and about 20:1, between about 1:1 and about 10:1, between about 1:1 and about 5:1, between about 2:1 and about 20:1, between about 2:1 and about 10:1, or between about 2:1 and about 5:1, or between about 3:1 and about 7:1, or between about 3:1 and about 4:1, or between about 6:1 and about 7:1. In certain implementations, the delivery vehicle complex comprises the cationic component and the nucleic acid at a cation:anion charge ratio of between about 2:1 and about 5:1. In still yet other implementations, the delivery vehicle complex comprises the cationic compound and the nucleic acid at a cation:anion charge ratio of about 3:1. In some implementations, the delivery vehicle complex comprises the cationic compound and the nucleic acid at a cation:anion charge ratio of about 3.7:1. In some implementations, the delivery vehicle complex comprises the cationic compound and the nucleic acid at a cation:anion charge ratio of about 6.4:1. In some implementations, the delivery vehicle complex comprises the cationic compound and the nucleic acid at a cation:anion charge ratio of about 4.8:1. In some implementations, the delivery vehicle complex comprises the cationic compound and the nucleic acid at a cation:anion charge ratio of about 7.2:1. In some implementations, the delivery vehicle complex comprises the cationic compound and the nucleic acid at a cation:anion charge ratio of about 3.6:1. In some implementations, the cationic component is a compound of Formula (I), such as a compound listed in Table 1. For example, the compound of Formula (I) can be compound 1, 6, 21, or 30.

Non limiting examples delivery vehicle compositions characterized by mass ratio and charge ratio can be found in Table 3, below.

TABLE 3
Mass and Charge Ratios of the Delivery Vehicle
System Components to Polyanionic Cargo

| | | Anionic or | Neutral | | |
|---|---|---|---|---|---|
| Mass | Cationic | Zwitterionic | Lipid | Shielding | Charge |
| Ratio | Component | Component | Component | Component | Ratio |
| F1A | 5.0 | 0 | 0 | 0.1 | 3.0 |
| F2A | 5.0 | 3.0 | 6.0 | 1.8 | 3.0 |
| F3A | 10 | 2.7 | 5.4 | 1.4 | 6.0 |
| F4A | 5.0 | 0 | 8.0 | 1.8 | 3.0 |
| F5A | 8.5 | 7.0 | 0 | 2.0 | 5.0 |
| F6A | 10 | 0 | 5.4 | 1.4 | 6.0 |
| F1 | 5.0 | 3.0 | 6.0 | 1.8 | 1.9 |
| F2 | 10 | 2.7 | 5.4 | 1.4 | 3.8 |
| F3 | 10 | 2.4 | 6.1 | 1.8 | 3.8 |
| F4 | 10 | 8.0 | 5.6 | 0.69 | 3.8 |
| F5 | 10 | 4.3 | 6.5 | 2.3 | 3.8 |
| F6/12 | 12 | 2.7 | 5.4 | 1.4 | 4.5 |
| F6/15 | 15 | 2.7 | 5.4 | 1.4 | 5.6 |
| F6/17 | 17 | 2.7 | 5.4 | 1.4 | 6.36 |
| F6.1 | 13 | 2.7 | 5.4 | 1.4 | 4.8 |
| F6.2 | 19 | 4.0 | 8.1 | 2.1 | 7.2 |
| F6.3 | 9.7 | 2.7 | 6.7 | 2.1 | 3.6 |
| D1 | 15 | 1.79 | 5.37 | 2 | — |
| D2 | 20 | 0 | 6.71 | 2 | — |
| D3 | 10 | 2.68 | 6.71 | 1.38 | — |
| D4 | 10 | 2.68 | 6.71 | 2 | — |
| D5 | 10 | 0 | 6.71 | 2 | — |
| D6 | 20 | 1.79 | 4 | 1.38 | — |
| D7 | 10 | 2.68 | 4 | 2 | — |
| D8 | 10 | 0 | 4 | 1.38 | — |
| D9 | 20 | 2.68 | 6.71 | 1.38 | — |
| D10 | 10 | 0 | 6.71 | 1.38 | — |
| D11 | 20 | 2.68 | 5.37 | 1.38 | — |
| D12 | 10 | 1.79 | 5.37 | 1.38 | — |
| D13 | 10 | 0 | 4 | 2 | — |
| D14 | 10 | 2.68 | 4 | 1.38 | — |
| D15 | 20 | 0 | 4 | 2 | — |
| D16 | 15 | 2.68 | 4 | 1.38 | — |
| D17 | 20 | 2.68 | 4 | 2 | — |
| D18 | 20 | 0 | 4 | 1.38 | — |
| D19 | 20 | 0 | 6.71 | 1.38 | — |
| D20 | 20 | 2.68 | 6.71 | 2 | — |
| D21 | 20 | 0 | 6 | 2 | — |
| D22 | 20 | 1.79 | 7.16 | 1.84 | — |
| D23 | 10 | 2.68 | 5.37 | 1.38 | — |
| D24 | 15 | 2.68 | 6.71 | 1.38 | — |
| D25 | 15 | 5 | 4 | 1 | — |
| D26 | 15 | 2 | 4 | 3 | — |
| D27 | 20 | 2 | 9 | 1 | — |
| D28 | 15 | 5 | 9 | 3 | — |
| D29 | 20 | 5 | 4 | 3 | — |
| D30 | 11.6 | 1.3 | 2.5 | 0.6 | — |
| D31 | 13 | 1.5 | 2.3 | 0.7 | — |
| D32 | 21 | 2.6 | 7.8 | 2.8 | — |

### Delivery Vehicle Complex Characterization

The delivery vehicle complexes disclosed herein can be characterized by various different parameters, such as particle size, polydispersity index, and percent encapsulation of cargo. In one implementation, a delivery vehicle complex resembles nanoparticle, including at least one polyanionic compound (described further below) being encapsulated by a delivery vehicle composition. In one implementation, such a complex is an mRNA nanoparticle including a delivery vehicle composition encapsulating at least one mRNA.

In some implementations, the delivery vehicle complexes disclosed herein can have a mean diameter of less than 300 nm, or less than 275 nm, or less than 250 nm, or less than 225 nm, or less than 200 nm, or less than 175 nm, or less than 150 nm, or less than 125 nm, or less than 100 nm, or less than 90 nm, or less than 80 nm, or less than 70 nm, or less than 60 nm, or less than 50 nm, or less than 40 nm. In some implementations, the delivery vehicle complexes disclosed herein can range in size from about 40 nm to about 200 nm in diameter, or from about 50 nm to about 175 nm, or from about 50 nm to about 200 nm, or from about 60 nm to about 150 nm, or from about 60 nm to about 100 nm, or from about 60 nm to about 90 nm, or from about 70 nm to about 125 nm, or from about 80 nm to about 100 nm, or from about 70 nm to about 90 nm, or from about 75 nm to about 95 nm, or from about 80 nm to about 110 nm, or from about 90 nm to about 125 nm, or from about 70 nm to about 90 nm. In another implementation, the complex may have a size of greater than about 100 nm in diameter—e.g., between about 105 nm and about 250 nm, between about 110 nm and about 220 nm, between about 150 nm and about 200 nm, between about 110 nm and about 200 nm. In one implementation, the complex may have a size of between about 105 nm and about 200 nm in diameter. In some cases, the delivery vehicle complex exhibits a particle size of about 40 nm to about 115 nm, or about 55 nm to about 95 nm, or about 70 to about 80 nm, or about 75 nm. In various cases, the delivery vehicle complex exhibits a particle size of about 135 nm to about 225 nm, or about 155 nm to about 195 nm, or about 170 to about 180 nm, or about 175 nm. In some cases, the particle size depends on the method used to prepare the complex (e.g., via a microfluidic device or by hand). The particle size/diameter can be determined by dynamic light scattering (DLS), and described in Example 3.

In some implementations, the delivery vehicle complexes of the disclosure exhibit a polydispersity index (PDI) of less than about 0.3, 0.25, 0.2, 0.19, 0.18, 0.17, 0.16, 0.15, 0.14, 0.13, 0.12, 0.11, or 0.10.

In some implementations, at least about 40%, 45%, 50%, 55%, 60%, 65%, 66%, 67%, 68%, 69%, 70%, 75%, 80%, 85%, 90%, 95%, 96%, 97%, 98%, or 99% of the nucleic acid (e.g., RNA) cargo is fully encapsulated in the delivery vehicle complexes. The percentage of mRNA encapsulated within the delivery vehicle complexes can be determined used a modified RiboGreen assay, as described in Example 3.

In some implementations, the delivery vehicle complexes of the disclosure retain at least 70%, or at least 80%, or at least 85%, or at least 90%, or at least 95%, or at least 96%, or at least 97%, or at least 98%, or at least 99%, or 100% of the polyanionic compound after storage at 4° C.-10° C. for at least for 10 days—e.g., at least 20 days, 30 days, 40 days, 50 days, 60 days, 70 days, or more. In one implementation, the complexes retain the aforementioned level of polyanionic compound f at 4° C. for 48 days. Further, in some cases, the delivery vehicle complexes of the disclosure retain at least 70%, or at least 80%, or at least 85%, or at least 90%, or at least 95%, or at least 96%, or at least 97%, or at least 98%, or at least 99%, or 100% of their original size after storage at 4° C. for at least 10 days—e.g., at least 20 days, 30 days, 40 days, 50 days, 60 days, 70 days, or more. In one implementation, the delivery vehicle complexes retain the aforementioned size after storage at 4° C. or 48 days.

The delivery vehicle complexes disclosed herein are well tolerated at high and low doses with no systemic toxicity or adverse events.

### Other Delivery Vehicle Complex Components

The delivery vehicle complex described herein can include further components to fine tune the complex for particular applications. Examples of components may include those that facilitate endosomal escape, including but not limited to, buffering amines or polyamines; nitrogen-containing heterocycle groups and/or nitrogen-containing heteroaryl groups such as imidazoles, pyrroles, pyridines, pyrimidines; maleic acid derivatives; or membrane-lytic peptides.

The delivery vehicle complex may also optionally comprise moieties on the surface of the system. Targeting moieties can be peptides, antibody mimetics, nucleic acids (e.g., aptamers), polypeptides (e.g., antibodies), glycoproteins, small molecules, carbohydrates, or lipids. Non-limiting examples of the targeting moiety include a peptide such as somatostatin, octreotide, LHRH, an EGFR-binding peptide, RGD-containing peptides, a protein scaffold such as a fibronectin domain, an aptide or bipodal peptide, a single domain antibody, a stable scFv, or a bispecific T-cell engagers, nucleic acid (e.g., aptamer), polypeptide (e.g., antibody or its fragment), glycoprotein, small molecule, carbohydrate, or lipid. The targeting moiety may be an aptamer being either RNA or DNA or an artificial nucleic acid; small molecules; carbohydrates such as mannose, galactose and arabinose; vitamins such as ascorbic acid, niacin, pantothenic acid, carnitine, inositol, pyridoxal, lipoic acid, folic acid (folate), riboflavin, biotin, vitamin B12, vitamin A, E, and K; a protein or peptide that binds to a cell-surface receptor such as a receptor for thrombospondin, tumor necrosis factors (TNF), annexin V, interferons, cytokines, transferrin, GM-CSF (granulocyte-macrophage colony-stimulating factor), or growth factors such as vascular endothelial growth factor (VEGF), hepatocyte growth factor (HGF), (platelet-derived growth factor (PDGF), basic fibroblast growth factor (bFGF), and epidermal growth factor (EGF).

The delivery vehicle complex may also optionally comprise small molecule drugs or other biologics incorporated into the delivery vehicle complex. Non-limiting examples include incorporating drugs that disrupt the blood-brain-barrier or enhance cellular uptake; drugs that affect intracellular trafficking or endosomal escape; or drugs that are immunomodulators to affect antigen presentation when the delivery vehicle complex is used in as a vaccine.

### Polyanionic Compound

The delivery vehicle complexes of the disclosure can comprise one or more polyanionic compounds (polyanionic cargo) that can be delivered by the complex to a target in vivo, such as a cell. The polyanionic compound can be complexed to the cationic component (e.g., a compound of Formula (I), such as compound 1, 6, 21, or 30) of the delivery vehicle complex via electrostatic interactions.

In some implementations, the polyanionic compound comprises a nucleic acid. Nucleic acids, as used herein, include naturally occurring nucleic acids (e.g., DNA, RNA, and/or hybrids thereof), as well as unnaturally occurring nucleic acids. Nonlimiting examples of unnatural amino acids are those that comprise an unnatural backbone, modified backbone linkages such as phosphorothioate, unnatural or modified bases, and/or unnatural and modified termini. Exemplary nucleic acids include genomic DNA, complementary DNA (cDNA), messenger RNA (mRNA), micro RNA (miRNA), small interfering RNA (siRNA), small activating RNA (saRNA), peptide nucleic acids (PNA), antisense oligonucleotides, ribozymes, plasmids, and immune stimulating nucleic acids.

In some implementations, the polyanionic compound comprises RNA. The RNA may be selected from the group consisting of chemically modified or unmodified RNA, single-stranded or double-stranded RNA, coding or non-coding RNA, mRNA, oligoribonucleotide, viral RNA, retroviral RNA, self-replicating (replicon) RNA (srRNA), tRNA, rRNA, immunostimulatory RNA, microRNA, siRNA, small nuclear RNA (snRNA), small-hairpin (sh) RNA riboswitch, RNA aptamer, RNA decoy, antisense RNA, a ribozyme, or any combination thereof. In some implementations, the nucleic acid cargo is RNA including but not limited to modified mRNAs, self-amplifying RNAs, and circular RNAs. In some implementations, the RNA comprises a coding RNA.

RNA is the usual abbreviation for ribonucleic acid. It is a nucleic acid molecule, i.e. a polymer consisting of nucleotide monomers. These nucleotides are usually adenosine monophosphate (AMP), uridine monophosphate (UMP), guanosine monophosphate (GMP) and cytidine monophosphate (CMP) monomers or analogues thereof, which are connected to each other along a so-called backbone. The backbone is formed by phosphodiester bonds between the sugar, i.e. ribose, of a first and a phosphate moiety of a second, adjacent monomer. The specific order of the monomers, i.e. the order of the bases linked to the sugar/phosphate-backbone, is called the RNA sequence. Usually RNA may be obtainable by transcription of a DNA sequence, e.g., inside a cell. In eukaryotic cells, transcription is typically performed inside the nucleus or the mitochondria. In vivo, transcription of DNA usually results in the so-called premature RNA (also called pre-mRNA, precursor mRNA or heterogeneous nuclear RNA) which has to be processed into so-called messenger RNA, usually abbreviated as mRNA. Processing of the premature RNA, e.g. in eukaryotic organisms, comprises a variety of different posttranscriptional modifications such as splicing, 5′-capping, polyadenylation, export from the nucleus or the mitochondria and the like. The sum of these processes is also called maturation of RNA. The mature messenger RNA usually provides the nucleotide sequence that may be translated into an amino acid sequence of a particular peptide or protein. Typically, a mature mRNA comprises a 5′-cap, optionally a 5′UTR, an open reading frame, optionally a 3′UTR and a poly(A) tail.

In addition to messenger RNA, several non-coding types of RNA exist which may be involved in regulation of transcription and/or translation, and immunostimulation. Within the present disclosure the term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA).

5′-CAP-Structure: A 5′-CAP is typically a modified nucleotide (CAP analogue), particularly a guanine nucleotide, added to the 5′ end of an mRNA molecule. In certain implementations, the 5′-CAP is added using a 5′-5′-triphosphate linkage (also named m7GpppN). Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures may be used in the context of the present disclosure to modify the RNA sequence of the present disclosure. Further modified 5′-CAP structures which may be used in the context of the present disclosure are CAP1 (additional methylation of the ribose of the adjacent nucleotide of m7GpppN), CAP2 (additional methylation of the ribose of the 2^{nd} nucleotide downstream of the m7GpppN), CAP3 (additional methylation of the ribose of the 3^{rd}nucleotide downstream of the m7GpppN), CAP4 (additional methylation of the ribose of the 4^{th}nucleotide downstream of the m7GpppN), ARCA (anti-reverse CAP analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In the context of the present disclosure, a 5′ cap structure may also be formed in chemical RNA synthesis or RNA in vitro transcription (co-transcriptional capping) using cap analogues, or a cap structure may be formed in vitro using capping enzymes (e.g., commercially available capping kits).

A cap analogue refers to a non-polymerizable di-nucleotide that has cap functionality in that it facilitates translation or localization, and/or prevents degradation of the RNA molecule when incorporated at the 5′ end of the RNA molecule. Non-polymerizable means that the cap analogue will be incorporated only at the 5′terminus because it does not have a 5′ triphosphate and therefore cannot be extended in the 3′ direction by a template-dependent RNA polymerase.

Cap analogues include, but are not limited to, a chemical structure selected from the group consisting of m7GpppG, m7GpppA, m7GpppC; unmethylated cap analogues (e.g., GpppG); dimethylated cap analogue (e.g., m2,7GpppG), trimethylated cap analogue (e.g., m2,2,7GpppG), dimethylated symmetrical cap analogues (e.g., m7Gpppm7G), or anti reverse cap analogues (e.g., ARCA; m7,2′OmeGpppG, m7,2′dGpppG, m7,3′OmeGpppG, m7,3′dGpppG and their tetraphosphate derivatives) The synthesis of N⁷-(4-chlorophenoxyethyl) substituted dinucleotide cap analogues has been described recently (.

A poly(A) tail also called “3′-poly(A) tail” or “Poly(A) sequence” is typically a long homopolymeric sequence of adenosine nucleotides of up to about 400 adenosine nucleotides, e.g. from about 25 to about 400, from about 50 to about 400, from about 50 to about 300, from about 50 to about 250, or from about 60 to about 250 adenosine nucleotides, added to the 3′ end of an mRNA. In certain implementations of the present disclosure, the poly(A) tail of an mRNA or srRNA is derived from a DNA template by RNA in vitro transcription. Alternatively, the poly(A) sequence may also be obtained in vitro by common methods of chemical synthesis without being necessarily transcribed from a DNA-progenitor. Moreover, poly(A) sequences, or poly(A) tails may be generated by enzymatic polyadenylation of the RNA.

A stabilized nucleic acid, typically, exhibits a modification increasing resistance to in vivo degradation (e.g. degradation by an exo- or endo-nuclease) and/or ex vivo degradation (e.g. by the manufacturing process prior to composition administration, e.g. in the course of the preparation of the composition to be administered). Stabilization of RNA can, e.g., be achieved by providing a 5′-CAP-Structure, a poly(A) tail, or any other UTR-modification. Stabilization can also be achieved by backbone-modification (e.g., use of synthetic backbones such as phosphorothioate) or modification of the G/C-content or the C-content of the nucleic acid. Various other methods are known in the art and conceivable in the context of the disclosure, to stabilize or otherwise improve the function of the nucleic acid. Provided herein, therefore, are polynucleotides which have been designed to improve one or more of the stability and/or clearance in tissues, receptor uptake and/or kinetics, cellular access, engagement with translational machinery, RNA half-life, translation efficiency, immune evasion, immune induction (for vaccines), protein production capacity, secretion efficiency (when applicable), accessibility to circulation, protein half-life and/or modulation of a cell's status, function and/or activity.

A 5′-UTR is typically understood to be a particular section of RNA. It is located 5′ of the open reading frame of the mRNA. In the case of srRNA, the open reading frame encodes the viral non-structural proteins while the sequence of interest is encoded in the subgenomic fragment of the viral RNA. Thus, the 5′UTR is upstream of nsP1 open reading frame. In addition, the subgenomic RNA of the srRNA has a 5′UTR. Thus, the subgenomic RNA containing a sequence of interest encoding a protein of interest contains a 5′UTR. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present disclosure, a 5′UTR corresponds to the sequence of a mature mRNA or srRNA which is located between the 5′-CAP and the start codon. In one implementations, the 5′-UTR corresponds to the sequence which extends from a nucleotide located 3′ to the 5′-CAP, and in certain implementations from the nucleotide located immediately 3′ to the 5′-CAP, to a nucleotide located 5′ to the start codon of the protein coding region and in some cases to the nucleotide located immediately 5′ to the start codon of the protein coding region. The nucleotide located immediately 3′ to the 5′-CAP of a mature mRNA or srRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present disclosure, the term “a 5′-UTR of a gene”, such as “a 5′-UTR of a NYESO1 gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

Generally, the term “3′-UTR” refers to a part of the nucleic acid molecule which is located 3′ (i.e. “downstream”) of an open reading frame and which is not translated into protein. Typically, a 3′-UTR is the part of an RNA which is located between the protein coding region (open reading frame (ORF) or coding sequence (CDS) and the poly(A) sequence of the mRNA. In the context of the present disclosure, the term 3′-UTR may also comprise elements, which are not encoded in the template, from which an RNA is transcribed, but which are added after transcription during maturation, e.g. a poly(A) sequence. A 3′-UTR of the RNA is not translated into an amino acid sequence.

With respect to srRNA, the 3′-UTR sequence is generally encoded by the viral genomic RNA, which is transcribed into the respective mRNA during the gene expression process. The genomic sequence is first transcribed into pre-mature mRNA. The pre-mature mRNA is then further processed into mature mRNA in a maturation process. This maturation process comprises 5′capping. In the context of the present disclosure, a 3′-UTR corresponds to the sequence of a mature mRNA or srRNA (and the srRNA subgenomic RNA), which is located between the stop codon of the protein coding region, preferably immediately 3′ to the stop codon of the protein coding region for the sequence of interest, and the poly(A) sequence of the mRNA. The term “corresponds to” means that the 3′-UTR sequence may be an RNA sequence, such as in the mRNA sequence used for defining the 3′-UTR sequence, or a DNA sequence, which corresponds to such RNA sequence. In the context of the present disclosure, the term “a 3′-UTR of a gene”, is the sequence, which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence (both sense and antisense strand and both mature and immature) of the 3′-UTR.

According to certain implementations of the disclosure, the RNAs for use in the delivery vehicle complexes herein comprise an RNA comprising at least one region encoding a peptide (e.g., a polypeptide), or protein, or functional fragment of the foregoing. As used herein, “functional fragment” refers to a fragment of a peptide, (e.g., a polypeptide), or protein that retains the ability to induce an immune response. In one implementations, the coding RNA is selected from the group consisting of mRNA, viral RNA, retroviral RNA, and self-replicating RNA. In some implementations, the RNA encodes a viral peptide (e.g., a viral polypeptide), a viral protein, or functional fragment of the foregoing. In various cases, the RNA encodes for a human papillomavirus (HPV) protein, a variant thereof, or a functional fragment of any of the foregoing. In some cases, the RNA encodes for a HPV E6 protein (or a variant thereof), a HPV E7 protein (or a variant thereof), a combination thereof, or a functional fragment of any of the foregoing. In some cases, the HPV protein is from HPV subtype HPV 16, 18, 31, 33, 35, 39, 45, 51, 52, 56, 58, 59, 66, and/or 68. In various cases, the HPV protein is from HPV subtype HPV 16 and/or 18. In some cases, the RNA encodes for a viral spike protein or a functional fragment thereof. In some cases, the RNA encodes for a SARS-Related coronaviruses (e.g., severe acute respiratory syndrome coronavirus-2, (SARS-CoV-2), severe acute respiratory syndrome coronavirus (SARS-CoV), Middle East respiratory syndrome coronavirus (MERS-CoV), human coronavirus 229E (HCoV-229E), human coronavirus 0C43 (HCoV-0C43), human coronavirus HKU1 (HCoV-HKU1), and/or human coronavirus NL63 (HCoV-NL63). In various implementations, the RNA encodes for a SARS-CoV spike(S) protein, a variant thereof, or a functional fragment any of the foregoing. In some cases, the RNA encodes for an influenza protein, a variant thereof, or a functional fragment of any of the foregoing. In various implementations, the RNA encodes for influenza hemagglutinin (HA), or a functional fragment thereof. In some implementations the influenza A virus, has HA of a subtype selected from the group consisting of H1, H2, H3, H4, H5, H6, H7, H8, H9, H10, H11, H12, H13, H14, H15, and H16. In various implementations, the influenza subtype is HA strain H1, H2, H3 or H5. In some implementations, the RNA encodes for a combination of the foregoing.

Contemplated viruses for which the RNA of the delivery vehicle complex can encode, include, but are not limited to: Influenza type A and type B, Poliovirus, Adenovirus, Rabies virus, Bovine parainfluenza 3, human respiratory syncytial virus, bovine respiratory syncytial virus, Canine parainfluenza virus, Newcastle disease virus, Herpes Simplex virus-1 and Herpes Simplex virus-2, human papillomavirus, hepatitis virus A, hepatitis virus B, hepatitis C, and human immunodeficiency virus, cytomegalovirus, Varicella-zoster virus, Epstein-Barr Virus, Kaposi's Sarcoma virus, Human herpesvirus-6, humanherpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simplex, Gammaherpesvirinae, Gallid alphaherpesvirus 1, Ebolavirus, Marburgvirus, Alphavirus, Flavivirus, Yellow Fever virus, Dengue virus, Japanese Enchephalitis virus, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis virus, Kyasanur Forest Disease virus, Alkhurma Disease virus, Omsk Hemorrhagic Fever virus, Hendra virus, Nipah virus, Rubeola virus, Rubella virus, Human parvovirus B19, Variola, Alphavirus, Molluscum contagiosum virus, Arenaviridae, Bunyaviridae, Filoviridae, Flaviviridae, Paramyxoviridae, Togaviridae, Flaviviruses, Colorado tick fever virus (coltivirus), coxsackievirus, Rotavirus, Norovirus, astrovirus, adenovirus, adenovirus, human metapneumovirus, rhinovirus or coronavirus, such as SARS-CoV, SARS-CoV-2, MERS-CoV, HCOV NL63, HKU1, 229E and OC43 human papillomavirus, Ebolavirus, Marburgvirus, Alphavirus, Flavivirus, Yellow Fever, Dengue Fever, Japanese Enchephalitis, West Nile Viruses, Zikavirus, Venezuelan Equine Encephalomyelitis virus, Chikungunya virus, Western Equine Encephalomyelitis virus, Eastern Equine Encephalomyelitis virus, Tick-borne Encephalitis, Kyasanur Forest Disease, Alkhurma Disease, Omsk Hemorrhagic Fever, Hendra virus, Nipah virus, Rubeola virus, Rubella virus, Human parvovirus B19, Human herpesvirus type 6, Varicella-zoster virus, Cytomegalovirus, Epstein-Barr Virus, Kaposi's Sarcoma virus, human herpesvirus-7, human herpesvirus-8, Macacine alphaherpesvirus 1, Canine herpesvirus, Equid alphaherpesvirus 1, Bovine alphaherpesvirus 1, Human herpesvirus 2, Virus del herpes simplex, Gammaherpesvirinae, Gallid alphaherpesvirus 1, Variola, Alphavirus, Molluscum contagiosum virus, Hepatitis Virus-A, Hepatitis Virus-B, Hepatitis-C, Hepatitis-D, Hepatitis-E, Polioviruses, Arenaviridae, Bunyaviridae, Filoviridae, Flaviviridae, Paramyxoviridae, or Togaviridae, Flaviviruses such as Zikavirus, Colorado tick fever virus (coltivirus), coxsackievirus, Rotavirus, Norovirus, astrovirus, adenovirus, adenovirus, influenza virus A, human metapneumovirus, rhinoviruses coronavirus, Varicellovirus, Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever Sicilian virus, Sapporo virus, SARS coronavirus 2, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, Zika virus, bovine herpesviruses, pseudorabies viruses, Adenoviridae, Bovine adenovirus BAdV-9=Human adenovirus C, Anelloviridae (proposed family), Torque teno virus TTV, Bornaviridae, Borna disease virus BDV, Bunyaviridae, Aino virus, Cache valley virus CVV, Crimean Congo haemorrhagic fever virus CCHF, Hantaan virus HTNV, Jamestown Canyon virus JCV, LaCrosse virus LACV, Puumala virus, Rift valley fever virus RVFV, Caliciviridae, Norovirus, San Miguel sea lion virus SMSV-5, Circoviridae, Bovine circovirus BCV=evolved strain of Porcine circovirus type 2 PCV-2, Coronaviridae, Bovine coronavirus BCoV-1, Bovine torovirus BtoV, Flaviviridae, Bovine viral diarrhea virus BVDV, Japanese encephalitis virus JEV, Kyasanur forest disease virus KFDV, Louping ill virus, Murray Valley encephalitis virus MVE, Saint Louis encephalitis virus SLEV, Tick borne encephalitis virus TBEV, Wesselsbron virus, West Nile virus (including Kunjin), Hepeviridae, Hepatitis E virus HEV, Herpesviridae, Bovine herpesvirus BHV-4, Equine herpesvirus EHV-1, Infectious bovine rhinotracheitis virus IBR=BHV-1, Pseudorabies virus PRV, Orthomyxoviridae, Dhori virus, Influenza A virus, Thogotovirus THOV, Papillomaviridae, Bovine papilloma virus BPV, Paramyxoviridae, Bovine parainfluenza virus BPIV3, Bovine respiratory syncytial virus BRSV, Peste-des-petits ruminants virus PPRV, Rinderpest virus RPV, Parvoviridae, Bovine adeno-associated virus BAAV, Bovine hokovirus BHoV, Picornaviridae, Bovine enterovirus BEV-1, BEV-2, Bovine kobuvirus BKV-1 U-1 strain, Encephalomyocarditis virus EMC, Foot and mouth disease virus FMDV, Seneca valley virus SWV, Polyomaviridae, Bovine polyomavirus BPyV, Poxviridae, Aracatuba virus, Bovine papular stomatitis virus BPSV, Cantagalo virus, Cowpox virus, Pseudocowpox virus PCPV, Vaccinia virus, Reoviridae, Banna virus BAV, Bluetongue virus BTV, Epizootic haemorrhagic disease virus EHDV, Liao Ning virus LNV, Reovirus, Rotavirus, Retroviridae, Bovine foamy virus BFV, Bovine leukemia virus BLV, Rhabdoviridae, Bovine ephemeral fever virus BEFV, Rabies virus, Vesicular stomatitis virus VSV, Togaviridae, Eastern equine encephalitis virus EEEV, Getah virus, Ross River virus RRV, Sindbis virus, Venezuelan equine encephalomyelitis virus VEE, Anelloviridae (proposed family), Torque teno virus TTV, Bunyaviridae, Crimean Congo haemorrhagic fever virus, CCHF, Hantaan virus HTNV, Jamestown Canyon virus JCV, LaCrosse virus LCV, Caliciviridae, Norovirus, San Miguel sea lion virus SMSV-5, Sapovirus, Circoviridae, Porcine circovirus PCV-1 & PCV-2, Coronaviridae, Bovine coronavirus BCoV-1, Severe acute respiratory syndrome virus SARS, Transmissible gastroenteritis virus TGEV, Filoviridae, Ebola Reston virus, Flaviviridae, Bovine viral diarrhea virus BVDV, Dengue virus, Ilheus virus, Japanese encephalitis virus JEV, Louping ill virus, Murray Valley encephalitis virus MVE, Powassan virus, Tick borne encephalitis virus TBEV, Wesselsbron virus, West Nile virus WNV (including Kunjin), Hepeviridae, Hepatitis E virus HEV, Herpesviridae, Infectious bovine rhinotracheitis virus IBR=BHV-1, Porcine cytomegalovirus PCMV (B. Potts personal communication), Pseudorabies virus PRV, Orthomyxoviridae, Avian influenza virus (H5N1), Porcine influenza virus (H1N1, H1N2), Paramyxoviridae, Bovine parainfluenza virus BPIV3, Menangle virus MENV, Nipah virus NiV, Peste-des-petits ruminants virus PPRV, Rinderpest virus RPV, Tioman virus TIOV, Parvoviridae, Porcine hokovirus PHOV, Porcine parvovirus PPV, Picornaviridae, Encephalomyocarditis virus EMC, Foot and mouth disease virus FMDV, Porcine enterovirus PEV-9 PEV-10, Seneca valley virus SVV, Swine vesicular disease virus SVDV, Reoviridae, Banna virus BAV, Reovirus, Rotavirus, Retroviridae, Porcine endogenous retrovirus PERV, Rhabdoviridae, Rabies virus, Vesicular stomatitis virus VSV, Togaviridae, Eastern equine encephalitis virus EEEV, Getah virus, Ross River virus RRV or Venezuelan equine encephalomyelitis VEE.

In some implementations, the RNA encodes for adenovirus, alphavirus, calicivirus (e.g., a calicivirus capsid antigen), coronavirus polypeptides, distemper virus, Ebola virus polypeptides, enterovirus, flavivirus, hepatitis virus (AE), herpesvirus, infectious peritonitis virus, leukemia virus, Marburg virus, orthomyxovirus, papilloma virus, parainfluenza virus, paramyxovirus, parvovirus, pestivirus, picorna virus (e.g., a poliovirus), pox virus (e.g., a vaccinia virus), rabies virus, reovirus, retrovirus, and rotavirus. In certain implementations, the RNA encodes for SARS-CoV-2, HPV (e.g., E6 and/or E7 from HPV16 and/or HPV18), or influenza (e.g., influenza hemagglutinin (HA).

In some implementations, the combined delivery of two or more particular nucleic acids together may be especially useful for therapeutic applications. For example, in some implementations, the one or more polyanionic cargo compounds includes a combination of sgRNA (single guide RNA) as a CRISPR sequence and mRNA encoding Cas9. In still further implementations, the nucleic acids may also be complexed with proteins such as with the CRISPR/Cas9 ribonucleoprotein complex. In some cases, the multicomponent delivery vehicle system complexes with one or more of a nucleic acid selected from DNA and RNA (e.g., an antigenic RNA and adjuvanting DNA, such as CpG).

### Polynucleotide Synthesis

Methods of making polynucleotides of a predetermined sequence are well-known. Solid-phase synthesis methods are known for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well.

Any method known in the art for making RNA is contemplated herein for making the RNAs. Illustrative methods for making RNA include but are not limited to, chemical synthesis and in vitro transcription.

In certain implementations, the RNA for use in the methods herein is chemically synthesized. Chemical synthesis of relatively short fragments of oligonucleotides with defined chemical structure provides a rapid and inexpensive access to custom-made oligonucleotides of any desired sequence. Whereas enzymes synthesize DNA and RNA only in the 5′ to 3′ direction, chemical oligonucleotide synthesis does not have this limitation, although it is most often carried out in the opposite, i.e. the 3′ to 5′ direction. In certain implementations, the process is implemented as solid-phase synthesis using the phosphoramidite method and phosphoramidite building blocks derived from protected nucleosides (A, C, G, and U), or chemically modified nucleosides.

In some implementations, modifications are included in the modified nucleic acid or in one or more individual nucleoside or nucleotide. For example, modifications to a nucleoside may include one or more modifications to the nucleobase, the sugar, and/or the internucleoside linkage. In some implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, 1-ethyl-pseudouridine-MP, 1-propyl-pseudouridine-MP, 1-(2,2,2-trifluoroethyl)-pseudouridine-MP, 2-amino-adenine-MP, xanthosine-MP, 5-bromo-cytidine-MP, 5-aminoallyl-cytidine-MP, or 2-aminopurine-riboside-MP.

In other implementations having at least one modification, the polynucleotide includes a backbone moiety containing the nucleobase, sugar, and internucleoside linkage of: pseudouridine-alpha-thio-MP, 1-methyl-pseudouridine-alpha-thio-MP, or 5-bromo-cytidine-MP. Nucleoside and nucleotide modifications contemplated for use in the present disclosure are known in the art.

To obtain the desired oligonucleotide, the building blocks are sequentially coupled to the growing oligonucleotide chain on a solid phase in the order required by the sequence of the product in a fully automated process. Upon the completion of the chain assembly, the product is released from the solid phase to the solution, deprotected, and collected. The occurrence of side reactions sets practical limits for the length of synthetic oligonucleotides (up to about 200 nucleotide residues), because the number of errors increases with the length of the oligonucleotide being synthesized. Products are often isolated by HPLC to obtain the desired oligonucleotides in high purity.

In certain implementations, RNA is made using in vitro transcription. The terms “RNA in vitro transcription” or “in vitro transcription” relate to a process wherein RNA is synthesized in a cell-free system (in vitro). DNA, particularly plasmid DNA, is used as template for the generation of RNA transcripts. RNA may be obtained by DNA-dependent in vitro transcription of an appropriate DNA template, which in certain implementations is a linearized plasmid DNA template. The promoter for controlling in vitro transcription can be any promoter for any DNA-dependent RNA polymerase. Particular examples of DNA-dependent RNA polymerases are the T7, T3, and SP6 RNA polymerases. A DNA template for in vitro RNA transcription may be obtained by cloning of a nucleic acid, in particular cDNA corresponding to the respective RNA to be in vitro transcribed, and introducing it into an appropriate vector for in vitro transcription, for example into plasmid DNA. In one implementations of the present disclosure, the DNA template is linearized with a suitable restriction enzyme, before it is transcribed in vitro. The cDNA may be obtained by reverse transcription of mRNA or chemical synthesis. Moreover, the DNA template for in vitro RNA synthesis may also be obtained by gene synthesis.

Methods for in vitro transcription are known in the art. Reagents used in the methods typically include: 1) a linearized DNA template with a promoter sequence that has a high binding affinity for its respective RNA polymerase such as bacteriophage-encoded RNA polymerases; 2) ribonucleoside triphosphates (NTPs) for the four bases (adenine, cytosine, guanine and uracil); 3) in some cases, a cap analogue as defined above (e.g. m7G(5′)ppp(5′)G(m7G)); 4) a DNA-dependent RNA polymerase capable of binding to the promoter sequence within the linearized DNA template (e.g. T7, T3 or SP6 RNA polymerase); 5) optionally a ribonuclease (RNase) inhibitor to inactivate any contaminating RNase; 6) optionally a pyrophosphatase to degrade pyrophosphate, which may inhibit transcription; 7) MgCl₂, which supplies Mg²⁺ ions as a co-factor for the polymerase; 8) a buffer to maintain a suitable pH value, which can also contain antioxidants (e.g. DTT), and/or polyamines such as spermidine at optimal concentrations.

### Methods of Making the Delivery Vehicle Complex

Components of the delivery vehicle complex can be prepared through a variety of physical and/or chemical methods to modulate their physical, chemical, and biological properties. These may involve rapid combination of the hydroxyalkyl-capped cationic peptoids, such as 2-aminopropane-1,3-diol-capped cationic peptoids in water or a water-miscible organic solvent with the desired polyanionic cargo compound (e.g., oligonucleotides or nucleic acids) in water or an aqueous buffer solution. These methods can include simple mixing of the components by pipetting, or microfluidic mixing processes such as those involving T-mixers, vortex mixers, or other chaotic mixing structures. In some implementations, the multicomponent delivery system is prepared on a microfluidic platform.

It is to be understood that the particular process conditions for preparing the delivery vehicle complexes described herein may be adjusted or selected accordingly to provide the desired physical properties of the complexes. For example, parameters for mixing the components of the delivery system complex that may influence the final compositions may include, but are not limited to, order of mixing, temperature of mixing, mixing speed/rate, flow rate, physical dimensions of the mixing structure, concentrations of starting solutions, molar ratio of components, and solvents used.

Formulation of the delivery vehicle complexes can be accomplished in many ways. In some cases, all components can be pre-mixed prior to addition of the nucleic acid cargo, which can result in a uniform distribution of components throughout the delivery particle.

In other cases, the components can be added sequentially to produce a core-shell type structure. For example, a cationic component could be added first to begin particle condensation, followed by a lipid component to allow the particle's surface to associate with target cells, followed by a shielding component to prevent particle aggregation. For example, the hydroxyalkyl-capped cationic peptoids, such as 2-aminopropane-1,3-diol-capped cationic peptoids can be premixed with the nucleic acid cargo to form a core structure. Then, the lipid components (such as lipid components comprising phospholipids and cholesterol) can be added to influence cell/endosomal membrane association. Because the shielding component is primarily useful on the outside of the multicomponent delivery system, this component can be introduced last, so that it does not disrupt the internal structure of the system, but rather provides a coating of the system after it is formed.

Additional components in the complexes and composition, such as the additional components of polymers, surface-active agents, targeting moieties, and/or excipients, may be admixed and combined with the rest of the components before, during, or after the principal components of the nucleic acid cargo, the cationic component, the lipid component and the shielding component have been combined.

Accordingly, also provided herein is a method of forming the delivery vehicle complex disclosed herein, comprising contacting the compound or salt of Formula (I) with the polyanionic compound. In some implementations, the method comprises admixing a solution comprising the compound or salt of Formula (I) with a solution comprising the polyanionic compound

### Pharmaceutical Formulations and Modes of Administration

Also provided herein are pharmaceutical compositions that include the delivery vehicle complexes of the disclosure, and an effective amount of one or more pharmaceutically acceptable excipients. An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females. As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The complexes of the disclosure can be administered to a subject or patient in a therapeutically effective amount. The complexes can be administered alone or as part of a pharmaceutically acceptable composition or formulation. In addition, the complexes can be administered all at once, as for example, by a bolus injection, multiple times, or delivered substantially uniformly over a period of time. It is also noted that the dose of the compound can be varied over time.

The delivery vehicle complexes disclosed herein and other pharmaceutically active compounds, if desired, can be administered to a subject or patient by any suitable route, e.g. orally, rectally, parenterally, (for example, intravenously, intramuscularly, or subcutaneously) intracisternally, intravaginally, intraperitoneally, intravesically, or as a buccal, inhalation, or nasal spray. The administration can be to provide a systemic effect (e.g. eneteral or parenteral). All methods that can be used by those skilled in the art to administer a pharmaceutically active agent are contemplated.

Compositions suitable for parenteral injection may comprise physiologically acceptable sterile aqueous or nonaqueous solutions, dispersions, suspensions, or emulsions, and sterile powders for reconstitution into sterile injectable solutions or dispersions. Examples of suitable aqueous and nonaqueous carriers, diluents, solvents, or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

These compositions may also contain adjuvants such as preserving, wetting, emulsifying, and dispersing agents. Microorganism contamination can be prevented by adding various antibacterial and antifungal agents, for example, parabens, chlorobutanol, phenol, sorbic acid, and the like. It may also be desirable to include isotonic agents, for example, sugars, sodium chloride, and the like. Prolonged absorption of injectable pharmaceutical compositions can be brought about by the use of agents delaying absorption, for example, aluminum monostearate and gelatin.

The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

When the composition of the disclosure are used as vaccines, it may comprise one or more immunologic adjuvants. As used herein, the term “immunologic adjuvant” refers to a compound or a mixture of compounds that acts to accelerate, prolong, enhance or modify immune responses when used in conjugation with an immunogen (e.g., neoantigens). Adjuvant may be non-immunogenic when administered to a host alone, but that augments the host's immune response to another antigen when administered conjointly with that antigen. Specifically, the terms “adjuvant” and “immunologic adjuvant” are used interchangeably in the present disclosure. Adjuvant-mediated enhancement and/or extension of the duration of the immune response can be assessed by any method known in the art including without limitation one or more of the following: (i) an increase in the number of antibodies produced in response to immunization with the adjuvant/antigen combination versus those produced in response to immunization with the antigen alone; (ii) an increase in the number of T cells recognizing the antigen or the adjuvant; and (iii) an increase in the level of one or more cytokines. Adjuvants may be aluminum based adjuvants including but not limiting to aluminum hydroxide and aluminum phosphate; saponins such as steroid saponins and triterpenoid saponins; bacterial flagellin and some cytokines such as GM-CSF. Adjuvants selection may depend on antigens, vaccines and routes of administrations.

In some implementations, adjuvants improve the adaptive immune response to a vaccine antigen by modulating innate immunity or facilitating transport and presentation. Adjuvants act directly or indirectly on antigen presenting cells (APCs) including dendritic cells (DCs). Adjuvants may be ligands for toll-like receptors (TLRs) and can directly affect DCs to alter the strength, potency, speed, duration, bias, breadth, and scope of adaptive immunity. In other instances, adjuvants may signal via proinflammatory pathways and promote immune cell infiltration, antigen presentation, and effector cell maturation. This class of adjuvants includes mineral salts, oil emulsions, nanoparticles, and polyelectrolytes and comprises colloids and molecular assemblies exhibiting complex, heterogeneous structures. In one example, the composition further comprises pidotimod as an adjuvant. In another example, the composition further comprises CpG as an adjuvant.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Use

The delivery vehicle complexes disclosed herein can be used to deliver the polyanionic compound of the complex (or cargo) to a cell. Accordingly, disclosed herein are methods of delivering a polyanionic compound, such as a nucleic acid (e.g., RNA) to a cell comprising contacting the cell with the delivery vehicle complex or pharmaceutical composition disclosed herein. In some implementations, the cell can be contacted in vitro. In some implementations wherein the cell is contacted in vitro, the cell is a HeLa cell. In other implementations wherein the cell is contacted in vivo, the multicomponent delivery system of the present disclosure is administered to a mammalian subject. A mammalian subject may include but is not limited to a human or a mouse subject. In yet other implementations wherein the cell is contacted ex vivo, the cell is obtained from a human or mouse subject. In some cases, the cell is a tumor cell. In some cases, the cell is a muscle cell.

In some implementations, the one or more polyanionic cargo compounds may be delivered for therapeutic uses. Non-limiting therapeutic uses include cancer, infectious diseases, autoimmune disorders, and neurological disorders. In certain implementations, the complex comprising the multicomponent delivery system and the polyanionic cargo compound is used as a vaccine. Genetic vaccination, or the administration of nucleic acid molecules (e.g., RNA) to a patient and subsequent transcription and/or translation of the encoded genetic information, is useful in the treatment and/or the prevention of inherited genetic diseases but also autoimmune diseases, infectious diseases, cancerous or tumor-related diseases as well as inflammatory diseases. Genetic vaccination is useful for treating or preventing coronavirus. The vaccine target of the majority of these entities is the coronavirus' spike(S) protein, a heavily glycosylated trimeric class I fusion protein that coats the outside of the virus and is responsible for host cell entry. The S protein of SARS-CoV-2 shares high structural homology with SARS-CoV-1 and contains several subunits vital for viral entry into host cells through the angiotensin converting enzyme 2 (ACE2) receptor, including the S1 domain, the S2 domain, and the receptor binding domain (RBD). Thus, the S protein and its subunits, as well as accessible peptide sequences within these domains, are attractive vaccine antigen targets. Further, genetic vaccination is particularly use in the treatment of cancer because cancer cells express antigens, tumors are generally not readily recognized and eliminated by the host, as evidenced by the development of disease

Vaccines. The delivery vehicle complexes of the disclosure are also useful as vaccines, in which the polyanionic compound is an RNA that may encode an immunogen, antigen or neoantigen. The immune system of a host provides the means for quickly and specifically mounting a protective response to pathogenic microorganisms and also for contributing to rejection of malignant tumors. Immune responses have been generally described as including humoral responses, in which antibodies specific for antigens are produced by differentiated B lymphocytes, and cell mediated responses, in which various types of T lymphocytes eliminate antigens by a variety of mechanisms. For example, CD4 (also called CD4+) helper T cells that are capable of recognizing specific antigens may respond by releasing soluble mediators such as cytokines to recruit additional cells of the immune system to participate in an immune response. CD8 (also called CD8+) cytotoxic T cells are also capable of recognizing specific antigens and may bind to and destroy or damage an antigen-bearing cell or particle. In particular, cell mediated immune responses that include a cytotoxic T lymphocyte (CTL) response can be important for elimination of tumor cells and cells infected by a microorganism, such as virus, bacteria, or parasite. The delivery vehicle complexes of the disclosure have been found to induce immune responses when one or more of the polyanionic compound of the complex encodes a viral peptide (e.g. a viral polypeptide), a viral protein, or functional fragment of the foregoing.

Thus, the disclosure includes methods for inducing an immune response in a subject in need thereof, comprising administering to the subject an effective amount of the delivery vehicle complex (e.g., formulated as an antigenic composition) of the disclosure. Also disclosed herein is a method of treating a viral infection in a subject in need thereof, comprising administering to the subject an effective amount of the delivery vehicle complex of the disclosure. In some implementations, the administering is by intramuscular, intratumoral, intravenous, intraperitoneal, or subcutaneous delivery.

In various implementations, administering the delivery vehicle complexes of the disclosure (e.g., formulated as a composition, pharmaceutical formulation, or antigenic composition) to a subject can result in an increase in the amount of antibodies (e.g., neutralizing antibodies) against the viral antigen that is produced in the subject relative to the amount of antibodies that is produced in a subject who was not administered the delivery vehicle complex. In some implementations, the increase is a 2-fold increase, a 5-fold increase, a 10-fold increase, a 50-fold increase, a 100-fold increase, a 200-fold increase, a 500-fold increase, a 700-fold increase, or a 1000-fold increase.

The immune response raised by the methods of the present disclosure generally includes an antibody response, preferably a neutralizing antibody response, maturation and memory of T and B cells, antibody dependent cell-mediated cytotoxicity (ADCC), antibody cell-mediated phagocytosis (ADCP), complement dependent cytotoxicity (CDC), and T cell-mediated response such as CD4+, CD8+. The immune response generated by the delivery vehicle complexes comprising RNA that encodes a viral antigen as disclosed herein generates an immune response that recognizes, and preferably ameliorates and/or neutralizes, a viral infection as described herein. Methods for assessing antibody responses after administration of an antigenic composition (immunization or vaccination) are known in the art and/or described herein. In some implementations, the immune response comprises a T cell-mediated response (e.g., peptide-specific response such as a proliferative response or a cytokine response). In some implementations, the immune response comprises both a B cell and a T cell response. Antigenic compositions can be administered in a number of suitable ways, such as intramuscular injection, intratumoral injection, subcutaneous injection, intradermal administration and mucosal administration such as oral or intranasal. Additional modes of administration include but are not limited to intravenous, intraperitoneal, intranasal administration, intra-vaginal, intra-rectal, and oral administration. A combination of different routes of administration in the immunized subject, for example intramuscular and intranasal administration at the same time, is also contemplated by the disclosure.

Cancer. Various cancers (e.g., cervical cancer) may be treated with the polyanionic cargo compounds delivered by the delivery vehicle complexes of the present disclosure. As used herein, the term “cancer” refers to any of various malignant neoplasms characterized by the proliferation of anaplastic cells that tend to invade surrounding tissue and metastasize to new body sites and also refers to the pathological condition characterized by such malignant neoplastic growths. Cancers may be tumors or hematological malignancies, and include but are not limited to, all types of lymphomas/leukemias, carcinomas and sarcomas, such as those cancers or tumors found in the anus, bladder, bile duct, bone, brain, breast, cervix, colon/rectum, endometrium, esophagus, eye, gallbladder, head and neck, liver, kidney, larynx, lung, mediastinum (chest), mouth, ovaries, pancreas, penis, prostate, skin, small intestine, stomach, spinal marrow, tailbone, testicles, thyroid and uterus.

As a non-limiting example, the carcinoma which may be treated may be Acute granulocytic leukemia, Acute lymphocytic leukemia, Acute myelogenous leukemia, Adenocarcinoma, Adenosarcoma, Adrenal cancer, Adrenocortical carcinoma, Anal cancer, Anaplastic astrocytoma, Angiosarcoma, Appendix cancer, Astrocytoma, Basal cell carcinoma, B-Cell lymphoma), Bile duct cancer, Bladder cancer, Bone cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, General, Germ cell tumor, Glioblastoma multiforme, Glioma, Hairy cell leukemia, Head and neck cancer, Hemangioendothelioma, Hodgkin lymphoma, Hodgkin's disease, Hodgkin's lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Nasal cavity cancer, Nasopharyngeal cancer, Neck cancer, Neuroblastoma, Neuroendocrine tumors, Non-Hodgkin lymphoma, Non-Hodgkin's lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Paget's disease, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pineoblastoma, Pituitary gland cancer, Primary central nervous system lymphoma, Prostate cancer, Rectal cancer, Renal cell cancer, Renal pelvis cancer, Rhabdomyosarcoma, Salivary gland cancer, Sarcoma, Sarcoma, bone, Sarcoma, soft tissue, Sarcoma, uterine, Sinus cancer, Skin cancer, Small cell lung cancer, Small intestine cancer, Soft tissue sarcoma, Spinal cancer, Spinal column cancer, Spinal cord cancer, Spinal tumor, Squamous cell carcinoma, Stomach cancer, Synovial sarcoma, T-cell lymphoma), Testicular cancer, Throat cancer, Thymoma/thymic carcinoma, Thyroid cancer, Tongue cancer, Tonsil cancer, Transitional cell cancer, Transitional cell cancer, Transitional cell cancer, Triple-negative breast cancer, Tubal cancer, Tubular carcinoma, Ureteral cancer, Ureteral cancer, Urethral cancer, Uterine adenocarcinoma, Uterine cancer, Uterine sarcoma, Vaginal cancer, and Vulvar cancer.

In some implementations, the delivery vehicle complexes of the disclosure are used to treat a cancer is selected from the group consisting of cervical cancer, head and neck cancer, B-cell lymphoma, T-cell lymphoma, prostate cancer, and lung cancer. In some implementations, the delivery vehicle complexes can be used to treat cervical cancer.

Infectious Diseases. In some implementations, the delivery vehicle complexes of the present disclosure is used to treat infectious diseases, such as microbial infection, e.g., a viral infection, a bacterial infection, a fungal infection, or a parasitic infection. Non-limiting examples of infectious diseases include hepatitis (such as HBV infection or HCV infection), RSV, influenza, adenovirus, rhinovirus, or other viral infections.

Autoimmune diseases. Various autoimmune diseases and autoimmune-related diseases may be treated with the delivery vehicle complexes of the present disclosure. As used herein, the term “autoimmune disease” refers to a disease in which the body produces antibodies that attack its own tissues. As a non-limiting example, the autoimmune disease may be Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia, Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenia purpura (ATP), Autoimmune thyroid disease, Autoimmune urticaria, Axonal & neuronal neuropathies, Balo disease, Behcet's disease, Bullous pemphigoid, Cardiomyopathy, Castleman disease, Celiac disease, Chagas disease, Chronic fatigue syndrome**, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed ryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia**, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenia purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosis, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopenia purpura (TTP), Tolosa-Hunt syndrome, Transverse myelitis, Ulcerative colitis, Undifferentiated connective tissue disease (UCTD), Uveitis, Vasculitis, Vesiculobullous dermatosis, Vitiligo, and Wegener's granulomatosis (now termed Granulomatosis with Polyangiitis (GPA).

Neurological diseases. Various neurological diseases may be treated with the delivery vehicle systems of the present disclosure. As a non-limiting example, the neurological disease may be Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Klüver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Post infectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Shortlasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

In jurisdictions that forbid the patenting of methods that are practiced on the human body, the meaning of “administering” of a composition to a human subject or patient shall be restricted to prescribing a controlled substance that a human subject or patient will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

### EXAMPLES

The following examples are provided for illustration and are not intended to limit the scope of the disclosure.

### Example 1—General Synthesis of Hydroxyalkyl Cationic Peptoids

To apply the delivery vehicles disclosed herein towards systemic expression of antibody-based drugs, the peptoid structure was optimized to maximize desired features, including but not limited to, hepatic selectivity and expression in mice. For secreted protein production, selective liver expression is favorable for producing high levels in circulating serum. A two-phase strategy can be applied to optimizing a peptoid-based delivery system. First, optimal ionizable groups were evaluated using a common lipophilic portion of the molecule, and then second the lipid portion were separately optimized with that ionizable group. This approach took advantage of the modularity of peptoid structure, which allows for the ionizable N-terminal domain to be easily modified when the peptoid core is constant allowing efficient optimization of the entire molecule without missing significant lead candidates since interactions between lipid and cationic portions are typically minimal.

General protocols for synthesizing cationic peptoids disclosed herein can be found in WO2020/069442 and WO2020/069445, each of which is incorporated herein by reference in its entirety. The following example describes the general protocol for synthesis of the cationic peptoids

All polymers were synthesized by the submonomer approach using bromoacetic acid and primary amines. An Fmoc-Rink amide resin was used as the solid support. The Fmoc group on the resin was deprotected with 20% (v/v) piperidine-dimethylformamide (DMF). The amino resin was then amidated with bromoacetic acid. The amidation was followed by amination of the α-carbon by nucleophilic displacement of the bromide with a primary amine. The two steps were successively repeated to produce the desired cationic peptide sequence.

All reactions and washings were performed at room temperature unless otherwise noted. Washing of the resin refers to the addition of a wash solvent (usually DMF or dimethylsulfoxide (DMSO)) to the resin, agitating the resin so that a uniform slurry was obtained, followed by thorough draining of the solvent from the resin. Solvents were removed by vacuum filtration through the fritted bottom of the reaction vessel until the resin appeared dry. In all the syntheses, resin slurries were agitated via bubbling argon up through the bottom of the fritted vessel.

Initial Resin Deprotection. A fritted reaction vessel was charged with Fmoc-Rink amide resin. DMF was added to the resin and this solution was agitated to swell the resin. The DMF was then drained. The Fmoc group was removed by adding 20% piperidine in DMF to the resin, agitating the resin, and draining the resin. 20% piperidine in DMF was added to the resin and agitated for 15 minutes and then drained. The resin was then washed with DMF, six times.

Acylation/Amidation. The deblocked amine was then acylated by adding bromoacetic acid in DMF to the resin followed by N,N-diisoprooplycarbodiimide (DIC) in DMF. This solution was agitated for 30 minutes at room temperature and then drained. This step was repeated a second time. The resin was then washed with DMF twice and DMSO once. This was one completed reaction cycle.

Nucleophilic Displacement/Amination. The acylated resin was treated with the desired primary or secondary amine to undergo nucleophilic displacement at the bromine leaving group on the α-carbon. This acylation/displacement cycle was repeated until the desired peptide sequence was obtained.

Peptide Cleavage from Resin. The dried resin was placed in a glass scintillation vial containing a teflon-coated micro stir bar, and 95% trifluoroacetic acid (TFA) in water was added. The solution was stirred for 20 minutes and then filtered through solid-phase extraction (SPE) column fitted with a polyethylene frit into a polypropylene conical centrifuge tube. The resin was washed with 1 mL 95% TFA. The combined filtrates were then lyophilized three times from 1:1 acetonitrile:water. The lyophilized peptide was redissolved to a concentration of 5 mM in 5% acetonitrile in water.

Purification and Characterization. The redissolved crude peptide was purified by preparative HPLC. The purified peptide was characterized by LC-MS analysis.

In one particular instance, aminolipidated peptoids were synthesized by the submonomer method with bromoacetic acid and N,N′-diisopropylcarbodiimide (DIC). Polystyrene-supported MBHA Fmoc-protected Rink amide (200 mg representative scale, 0.64 mmol/g loading) resin was used as a solid support. For bromoacetylation, resin was combined with a 1:1 mixture of 0.8 M bromoacetic acid and 0.8 M N,N′-diisopropylcarbodiimide (DIC) for 15 minutes. Amine displacement was carried out using a 1M solution of amine in DMF for 45 minutes. Following synthesis, crude peptoids were cleaved from resin using 5 mL of a mixture of 95:5 trifluoroacetic acid (TFA): water for 40 minutes at room temperature. Resin was removed by filtration and the filtrate was diluted in water to form a milky white suspension, followed by addition of polystyrene resin for (heterogeneous) solid phase extraction of peptoids. The solution was mixed well and incubated for 20 min at room temperature. Next, the solid phase extracted peptoids were released by addition of ethanol and concentrated using a vacuum centrifuge. The crude peptoids were further purified by reverse-phase flash chromatography using a C4 column and a gradient from 60-95% ACN/H₂O containing 0.1% TFA. Purity and identity were assayed with LC-MS system consisting of a TOF mass spectrometer (LC/TOF). Chromatographic separation was performed using a C8 column (2.1×100 mm) at 40° C. The gradient system was used at a flow rate of 0.2 ml/min: initially, the mobile phase consisted of acetonitrile-water-formic acid (60:40:0.1) containing 10 mM ammonium formate; then it was programmed in a linear manner to isopropanol-acetonitrile-formic acid (80:20:0.1) containing 10 mM ammonium formate over 11 min.

For this screen, a lipid block containing 4 dodecyl lipid (N-dodecylglycine) monomers was selected and 12 peptoids containing these groups were synthesized on a solid-phase synthesizer using the submonomer approach. The resulting materials were purified, and their identity was confirmed by LC-MS. It was observed that changing both the number of lipid monomers, and total lipid carbons had a significant impact on the hydrophobicity of peptoid molecules as measured by HPLC retention time. When fit to a multi-parameter model, as the number of lipid carbons increases, both the calculated logP and empirical reversed-phase HPLC retention time increase, as would be expected for a molecule with increasing lipid content. Additionally, as the number of lipid monomers increases (at a similar number of total lipid carbons), the predicted logP and retention time decrease, likely due to the increased contribution of hydrophilic amide backbone towards the overall molecule polarity (i.e. a peptoid with 6×6-carbon lipid monomers is much more hydrophilic than one with 3×12-carbon monomers). Taken together this these parameters resulted in a library of peptoids with varying physical properties to be used for optimization.

Characterization data for peptoids disclosed herein are provided in Table 4.

TABLE 4
Peptoid characterization data

| | HPLC | | |
|---|---|---|---|
| | Retention | Calculated | Found |
| | Time | Exact | Exact |
| Compound | (min) | Mass | Mass |
| 4 | 6.478 | 905.79 | 906.80 |
| 6 | 7.499 | 1157.02 | 1158.02 |
| 7 | 6.837 | 959.84 | 960.85 |
| 8 | 8.163 | 1669.45 | 1671.46 |
| 9 | 7.804 | 1609.35 | 1611.36 |
| 10 | 6.042 | 1333.07 | 1334.08 |
| 11 | 7.103 | 1501.26 | 1502.26 |
| 12 | 5.899 | 1106.9 | 1107.91 |
| 13 | 6.455 | 1389.13 | 1390.14 |
| 14 | 5.7 | 1277.01 | 1278.02 |
| 15 | 7.33 | 1501.26 | 1202.06 |
| 16 | 8.396 | 1499.34 | 1501.37 |
| 17 | 8.186 | 1669.45 | 1671.82 |
| 20 | 6.583 | 931.81 | 932.82 |
| 21 | 6.799 | 1048.92 | 1049.94 |
| 22 | 6.389 | 992.86 | 993.86 |
| 23 | 6.121 | 936.8 | 1107.91 |
| 24 | 5.278 | 824.67 | 825.68 |
| 25 | 5.815 | 1106.9 | 1107.91 |
| 26 | 5.891 | 1219.03 | 1220.04 |
| 27 | 7.651 | 1387.22 | 1388.23 |
| 28 | 6.587 | 1219.03 | 1220.04 |
| 29 | 6.943 | 1275.09 | 1276.10 |
| 30 | 7.73 | 1387.22 | 1388.23 |
| 31 | 7.052 | 1275.09 | 1276.11 |
| 32 | 6.179 | 1162.96 | 1163.98 |
| 33 | 7.743 | 1387.22 | 1388.23 |
| 34 | 6.622 | 1219.03 | 1220.04 |
| 35 | 6.977 | 1275.09 | 1276.11 |
| 37 | 6.096 | 936.8 | 937.801 |
| 40 | 9.686 | 1387.22 | 1388.22 |
| 41 | 7.359 | 1275.09 | 1276.10 |
| 43 | 7.039 | 1275.09 | 1276.10 |
| 44 | 7.048 | 1100.95 | 1101.97 |
| C1 | 6.627 | 1017.93 | 1018.93 |
| C2 | 6.922 | 1032.93 | 1033.93 |
| C3 | 6.939 | 1018.91 | 1019.92 |
| C4 | 6.901 | 1062.94 | 1063.94 |
| C5 | 6.602 | 1031.94 | 1032.95 |
| C6 | 6.96 | 1032.93 | 1033.94 |
| C7 | 6.971 | 1046.94 | 1047.95 |
| C8 | 6.537 | 1082.95 | 1083.94 |
| C9 | 6.437 | 1096.97 | 1116.02 |
| C10 | 6.438 | 1101.98 | 1102.98 |
| C11 | 6.892 | 1100.01 | 1048.10 |
| C12 | 6.606 | 1046.94 | 1101.01 |
| C13 | 6.579 | 1045.96 | 1048.102 |

### Example 2—Synthesis of Delivery Vehicle Complexes

Synthesis. The hydroxyalkyl-capped cationic peptoids, such as 2-aminopropane-1,3-diol-capped cationic peptoids can be evaluated for therapeutic and/or prophylactic purposes in vitro or in vivo. Without being bound to any particular theory, the cationic portion(s) of the amino-lipidated peptoids bind to the negatively-charged phosphodiester backbone of the polyanionic cargo (e.g., nucleic acid cargo) through primarily electrostatic interactions, forming a mixed coacervate complex. Hydrophobic interactions between lipid chains on the hydroxyalkyl-capped cationic peptoids, such as 2-aminopropane-1,3-diol-capped cationic peptoids can act to stabilize particle formation and assist with membrane association.

Delivery vehicle complexes can be prepared through any physical and/or chemical methods known in the art to modulate their physical, chemical, and biological properties. These methods typically involve rapid combination of the hydroxyalkyl-capped cationic peptoids, such as 2-aminopropane-1,3-diol-capped cationic peptoids in water, or a water-miscible organic solvent, with the oligonucleotide in water or an aqueous buffer solution. These methods can include simple mixing of the components by pipetting, or microfluidic mixing processes such as those involving T-mixers, vortex mixers, or other chaotic mixing structures.

In standard formulations, the hydroxyalkyl-capped cationic peptoids, such as 2-aminopropane-1,3-diol-capped cationic peptoids and additional lipids were dissolved in anhydrous ethanol at a concentration of 10 mg/mL to result in solutions that are stable at room temperature. In some implementations, the solutions were stored at −20° C. The nucleic acid cargo was dissolved in DNAse or RNAse-free water at a final concentration of 1-2 mg/mL. These solutions can be stored at −20° C. or −78° C. for extended time periods.

To prepare the delivery vehicle compositions disclosed herein, hydroxyalkyl-capped cationic peptoids, such as 2-aminopropane-1,3-diol-capped cationic peptoids and additional lipid components were first pre-mixed in an ethanol phase at the required mass ratios. Nucleic acid cargo(s) were diluted in ethanol and acidic buffer (10 mM phosphate/citrate, pH 5.0). Ethanol and aqueous phases were mixed at a 3:1 volume ratio, and then immediately diluted with a 1:1 volume ratio of PBS, resulting in a final mRNA concentration of 0.1 μg/μL. Non-liming exemplary delivery vehicle compositions prepared by the aforementioned method include the compositions listed in Table 2, above (e.g., compositions F2, F6/17, F6/12, and F6/15).

The delivery vehicle compositions were combined with a polyanionic compound, such as an RNA encoding for, e.g., firefly luciferase (Fluc), a COVID-19 spike protein a functional fragment thereof, and/or a variant thereof, or the E6/E7 oncogene (e.g., from HPV16, HPV18, a functional fragment thereof, and/or a variant thereof) at the ratios indicated in Table 3 to form delivery vehicle complexes to be evaluated for therapeutic and/or prophylactic purposes in vitro or in vivo. The w/w in Table 3 is the ratio of the indicated component to the mRNA by mass.

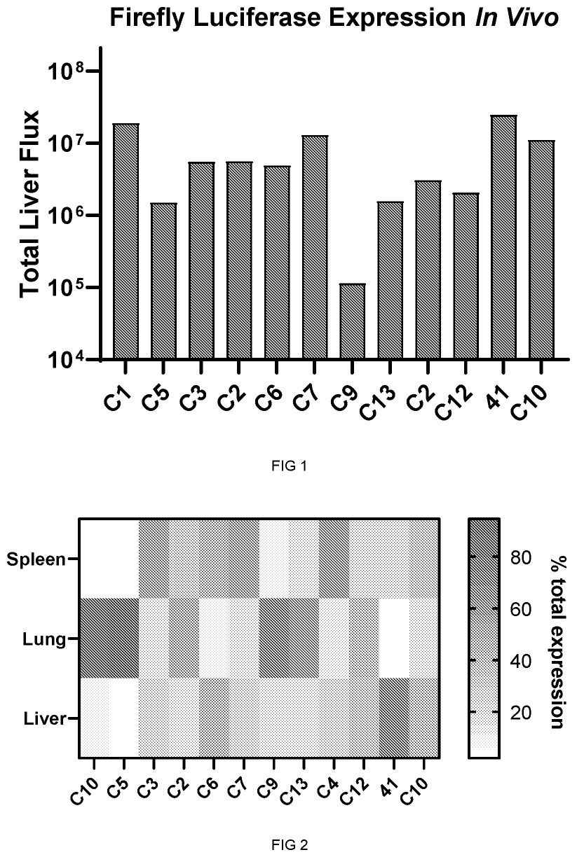

In one particular instance, peptoids disclosed herein were formulated into delivery vehicles by first dissolving in ethanol along with DSPC, cholesterol, and DMG-PEG2000 at weight ratio of 20:1.8:7.2:1.8 (Formulation D22 in Table 3, above), then mixed with firefly luciferase (fLuc) mRNA dissolved in an aqueous buffer at a volume ratio of 1:3 via microfluidic nanoprecipitation on a turbulent mixing device, resulting in small, low dispersity particles. Physical particle properties were characterized. Delivery vehicles with initial sizes larger than 120 nm were not brought forward into in vivo screening as liver fenestrae primarily accommodate LNPs sized under 100 nm. In vivo expression of delivery vehicles comprising peptoids with varied headgroups was evaluated using fLuc expression in BALB/c mice 6 hours after IV dose (FIG. 1). This panel of headgroup-modified peptoids showed significant differences in both total expression levels as well as organ selectivity, highlighting the importance of screening wide chemical diversity in the ionizable portion of the peptoid. The aminoethyl and aminopropyl headgroups both show preferential expression in lung (FIG. 2), potentially due to the high basicity of the pendant primary amine, as groups with higher pKas are known to accumulate in that organ. Interestingly, the piperizinyl propyl and methoxypropyl head groups shifted selectivity towards spleen. These results provide evidence that simply tuning peptoid headgroup leads to dramatic shifts in selective expression. While spleen and lung selectivity merit further optimization for future therapeutic applications, liver-selective targets were used for further studies herein to prepare for systemic delivery of antibody targets and maximize their serum expression with anti-RSV cargo. The 1,3 diol (apd) head group shows the highest liver expression and selectivity among all of the tested peptoids, and was chosen for further lipid block optimization.

mRNA was transcribed from DNA template encoding firefly luciferase using HiScribe® T7 high yield RNA synthesis kit. N¹-Methylpseudo-UTP (m1ΨTP) was used in place of UTP for the reaction and CleanCap® was used for co-transcriptional capping during IVT. NTP concentrations used were calculated according to the template sequence and 4:1 Cap to GTP ratio was used for the IVT. The reaction was performed for 4 hours at 37° C.

The DNA template was removed through TURBO™ DNase digestion at 0.15 U/μL reaction volume for 30 min at 37° C. The transcript was then purified using RNAClean™ XP beads. The RNA-bound beads were washed 3 times with freshly prepared 70% ethanol, air dried for 15 minutes followed by elution in 200 μL of nuclease-free water. The purified mRNA was quantified spectrophotometrically and analyzed using a Labchip® GX Touch™ nucleic acid analyzer.

Delivery vehicles were formulated by rapid microfluidic mixing using a high throughput microfluidic system. Briefly, ethanol solutions containing peptoid, DSPC, Cholesterol, and DMG-PEG2000 at a weight ratio of 20:1.79:7.16:1.84 (Formulation D22 from Table 3, above), were mixed with 10 mM sodium citrate buffer (pH 5.0) containing synthesized mRNA at a ratio of 1:3 by volume. Peptoid-based LNPs were subsequently dialyzed against 1-2 liter tris-sucrose buffer (TS7, pH 7.4) overnight at 4° C.

Apd lipid block variation peptoids were formulated into delivery vehicles and physical properties were characterized and analyzed. Overall, it was observed that monomer length and total carbon number have significant impacts on physical properties of particles, and that the longest monomer and maximum total carbon are not the top performing suggesting that the range of monomers lengths tested was sufficient. Particle size was found to be difficult to correlate to any single factor, except that particles were significantly larger when the oleyl (Ole) monomer was used relative to 2-ethylhexyl (Ehx). Percent of encapsulated mRNA significantly depended on both the total number of lipid carbons on the peptoids, and the type of branched/unsaturated monomer used, with maximal encapsulation occurring around 55 total lipid carbons, and using the 2-ethylhexyl (ehx) monomer. It was observed that particle pKa varies from pH 5-7 with higher monomer and high total carbon count leading to lower particle pKa. Additionally, it was observed that increasing the number of branched monomers leads to a slightly lower pKa in delivery vehicles, even with similar headgroup pKa. While traditional LNP materials achieve the optimal particle pka of 6-6.5 by utilizing a tertiary or secondary amine with predicted pKa of 9.5-10.5, Apd peptoids have predicted pKas of approximately 5.5-5.7, which are very similar to their measured particle pKas of 5-7, indicating a lack of pKa shift upon formulation into a lipid nanoparticle complex. This could be due to several factors including the hydrophilicity of the peptoid backbone providing an environment more favorable to amine protonation, or the packing dynamics of the of the peptoid within the particle, and will be the focus of further investigation. Fusogenicity, or the ability of the LNP to fuse with endosomal membranes and escape degradation, has also been shown to vary with lipid structure for LNPs. Hemolysis of RBC at neutral and acidic pH was used as a measure of fusogenicity. At low pH which is most predictive of membrane interactions in the endosome, increasing branched/unsaturated groups leads to higher hemolysis at pH 5 for peptoids with overall lower total monomers. Interestingly, it was observed find that introduction of oleyl lipids often increased hemolysis at both pH 5 and 7. This suggests that oleyl based peptoids may not be well tolerated due to background levels of hemolysis at neutral pH.

### Example 3—Particle Characterization

The resulting delivery vehicle complexes were evaluated by dynamic light scattering (DLS) to determine the volume average particle size/diameter (nm) and the size polydispersity index (PDI) within the delivery vehicle complex.

Particle size. Particle sizes and size distributions were measured using a Wyatt DynaPro® Plate Reader III. In general, formulated samples were diluted to 2 ng/μL in 100 UL PBS. Data is reported as the hydrodynamic diameter (in nm) of the cumulant fit of the correlation function, and the polydispersity of that measurement.

Percent encapsulation. The percentage of mRNA encapsulated within the delivery vehicle complexes containing Fluc mRNA was determined using a modified RiboGreen® assay. In general, formulated mRNA samples were diluted to 500 ng/ml in Tris-EDTA buffer with or without Triton-X™. RiboGreen® (Invitrogen) was added at a 200-fold dilution, and the plate was incubated for 5 minutes. Fluorescence was measured at Ex. 840 nm/Em. 520 nm and encapsulated mRNA was calculated by taking the ratio of fluorescence for non-lysed particles versus lysed particles. Complexes with increased amounts of cationic component resulted in higher encapsulation.

Particle Stability. The delivery vehicle complexes were stored at 4° C., and the resulting particle size and percent encapsulation were determined after 17 days and 48 days.

In one particular instance, the size and polydispersity (PDI) of delivery vehicles was measured by dynamic light scattering in PBS using a DynaPro® plate reader III. The zeta potential of delivery vehicles was determined using Nano-ZS™ particle analyzer. Zeta sample was diluted to 30 uM total lipid in a weakly buffered solution of 10 mM HEPES, 10 mM MES, 10 mM ammonium acetate pH 7. Measurements were collected in duplicate for over 30 run with the voltage manually set to 120 mV

The mRNA concentration and the percentage of encapsulated mRNA were determined using a fluorescence-based Quant-iT™ RiboGreen® RNA Reagent Kit using a literature procedure. To determine the mRNA concentration, delivery vehicles were incubated with 2% Triton™ X-100 for 10 min to release entrapped mRNA, followed by addition of RiboGreen® RNA reagent. The fluorescence intensity of mRNA-RiboGreen® reagent complex (excitation at 485 nm and emission at 530 nm) was detected using a microplate reader. The ratio of fluorescence without and with Triton™ X-100 were used to calculate the percentage of encapsulated mRNA.

The retention time and the purity of synthesized peptoids were detected using HPLC-UV, with the UV absorption at 214 nm and injection volume (3 μL). RP-HPLC was performed using a 1.7 μm 4 protein column (pore size: 300 Å, 2.1 (I.D.)×100 mm) via a running gradient at a temperature of 55° C. with a mixture of Solvent A and Solvent B (Solvent A: water/isopropanol (98/2 with 0.1% v/v trifluoroacetic acid), Solvent B: isopropanol/acetonitrile (80/20 with 0.1 trifluoroacetic acid)) from 35 to 90% solvent B in 8 min, followed by a 4 min wash with 100% solvent B and return to 35% solvent B at a flow rate of 0.4 ml/min.

The apparent particle pKa was assessed by a TNS fluorescence assay. Briefly, particles were diluted to 130 uM total lipid in TNS buffer ranging in pH 3-11 for 10 minutes with 10 mM of TNS probe. The fluorescence was measured at room temperature in a plate reader ex 320/em 445. Fluorescence intensity was normalized to the minimal and maximal values and the pKa was determined by a 3 parameter fit to determine the half maximum of the curve.

RBC hemolysis was used as a metric for fusogenicity of LNPs at low pH. Sheep RBCs were washed with 2×PBS by pelleting at 800×g and resuspended to 5% v/v in either 20 mM citrate 140 mM NaCl PH 5.5 or PBS. Particles were diluted to 150 mM total lipid in well with 5% RBC solution and incubated at 37° C. for 45 min. Supernatant was collected and absorbance read on a microplate reader at 425 nm. Fusion was normalized to a fully lysed sample with 2% Triton™ and a buffer-only control. % hemolysis=100*[(ABS T-background)−(ABS sample-background)/(ABS T-background)].

Characterization data for delivery vehicles comprising peptoids disclosed herein are presented in Table 5. Characterization data for delivery vehicles comprising comparative compounds C1-C13 are presented in Table 6.

TABLE 5
Delivery vehicle characterization data

| | Particle | | | | | | |
|---|---|---|---|---|---|---|---|
| | Size | | % | Zeta | pKa | Hemolysis | Hemolysis |
| Compound | (nm) | PDI | Encapsulation | Potential | (TNS) | at pH 5 | at pH 7 |
| 4 | 631.3 | Multimodal | 12.3 | | | | |
| 5 | | | | | | | |
| 6 | 71.3 | 0.39 | 85.9 | −0.785 | 6.373 | 21.5 | 60.6 |
| 7 | 62.3 | 0.13 | 86.3 | −2.31 | 6.283 | 6.1 | 23.8 |
| 8 | 90.5 | 0.04 | 46.6 | −12.4 | 5.786 | 0.5 | −0.1 |
| 9 | 94.9 | 0.14 | 95.2 | −1.6 | 5.906 | 1.1 | 0.1 |
| 10 | 122.5 | 0.3 | 97.0 | −2.71 | 6.052 | 1.1 | 0.0 |
| 11 | 106.6 | 0.111 | 66.1 | | | | |
| 12 | 91.4 | 0.32 | 97.4 | −8.68 | 6.018 | 52.1 | 1.5 |
| 13 | 122.4 | 0.102 | 82.0 | −5.17 | 5.686 | 1.8 | 0.6 |
| 14 | 2228.3 | Multimodal | 57.3 | −3.14 | 6.825 | 9.1 | 0.2 |
| 15 | 106.6 | 0.111 | 84.5 | −8 | 5.909 | 0.9 | 0.6 |
| 16 | 93.1 | 0.24 | 30.6 | −3.58 | 5.554 | 4.1 | 2.2 |
| 17 | 96.7 | 0.11 | 50.3 | −17.8 | 5.656 | 2.1 | 0.3 |
| 20 | 65.4 | 0.18 | 70.1 | 3.41 | 5.773 | 16.7 | 33.3 |
| 21 | 66.4 | 0.262 | 81.2 | 2.7 | 5.406 | 35.8 | 59.5 |
| 22 | 51.5 | 0.179 | 83.3 | 4.24 | 5.888 | 58.9 | 81.5 |
| 23 | 1269.5 | Multimodal | 2.3 | | | | |
| 24 | 66.9 | 0.12 | 97.6 | −1.59 | 6.43 | 100.4 | 99.9 |
| 25 | 2220.3 | Multimodal | 24.3 | | | 94.6 | 94.7 |
| 26 | 421.9 | Multimodal | 66.1 | | | | |
| 27 | 107.6 | 0.214 | 87.9 | 1.84 | 5.48 | 12.4 | 9.1 |
| 28 | 86.6 | 0.089 | 82.2 | −5.98 | 5.844 | 15.1 | 5.6 |
| 29 | 92.7 | 0.094 | 81.9 | −7.04 | 6.044 | 4.8 | 1.3 |
| 30 | 82.6 | 0.097 | 87.7 | −1.41 | 6.084 | 17.3 | 2.1 |
| 31 | 80.8 | 0.1 | 86.9 | −11.2 | 6.523 | 5.8 | 2.0 |
| 32 | 692.4 | Multimodal | 23.3 | | | | |
| 33 | 165.8 | 1 | −1.8 | −2.12 | 5.935 | 0.9 | 0.4 |
| 34 | 118.4 | 0.093 | 71.2 | −9.58 | 6.374 | 11.8 | 27.6 |
| 35 | 90.7 | 0.082 | 76.1 | −5.88 | 5.937 | 4.5 | 5.1 |
| 37 | 181.4 | Multimodal | 56.4 | −0.676 | 6.418 | 7.8 | 23.9 |
| 40 | 270.5 | Multimodal | 55.9 | −7.48 | 4.883 | 0.5 | −0.1 |
| 41 | 75.7 | 0.15 | 94.2 | −5.38 | 6.068 | 4.5 | 2.9 |
| 43 | 2058.2 | Multimodal | | −6.37 | 4.595 | 1.2 | 0.2 |
| 44 | 98.1 | 0.194 | 76.4 | 2.39 | 6.936 | 51.6 | 95.8 |

TABLE 6
Comparative peptoid delivery vehicle characterization data.

| | Particle | | | Hemolysis | Hemolysis |
|---|---|---|---|---|---|
| | Size | | % | at pH | at pH |
| Compound | (nm) | PDI | Encapsulation | 5 | 7 |
| C1 | 61.3 | 0.206 | 86.6 | −15.1 | 39.2 |
| C2 | 67.0 | 0.179 | 92.2 | 10.6 | 37.2 |
| C3 | 65.3 | 0.196 | 87.5 | 24.4 | 51.1 |
| C4 | 67.7 | 0.186 | 90.5 | 4.9 | 12.0 |
| C5 | 57.3 | 0.135 | 97.5 | −15.6 | 39.8 |
| C6 | 67.1 | 0.223 | 92.6 | 4.9 | 22.7 |
| C7 | 71.3 | 0.219 | 93.1 | 31.6 | 65.5 |
| C8 | 60.2 | 0.192 | 93.5 | 2.3 | 8.1 |
| C9 | 63.0 | 0.201 | 98.8 | 0.7 | 2.4 |
| C10 | 84.6 | 0.520 | 86.6 | 0.6 | 1.0 |
| C11 | 70.2 | 0.179 | 92.4 | 9.6 | 10.2 |
| C12 | 62.4 | 0.175 | 95.9 | −2.4 | 3.9 |
| C13 | 57.6 | 0.180 | 95.4 | −12.3 | 8.2 |

### Example 4—Delivery Vehicle Complex Efficacy—In Vitro Firefly Luciferase Expression

The efficacy of complexes comprising the delivery vehicle compositions disclosed herein and mRNA encoding for firefly luciferase (Fluc mRNA) is evaluated in vitro based on their ability to deliver the firefly luciferase (Fluc) reporter gene to cultured cells. In a representative experiment, the delivery vehicle compositions were combined with Fluc mRNA, and the resulting particles were added to cultured HEK-293 cells at a dose of 50 ng/well (in 150 μL total volume). The resulting luciferase expression was measured by a luminescence plate reader after 6 hours and 18 hours of treatment. The delivery vehicles of the disclosure showed high luciferase expression.

### Example 5—In Vivo Luciferase Expression

General methods. All animal studies were conducted by Lumigenics LLC (Hercules, CA) under accreditation by the California State Department of Public Health and under supervision of IACUC and Veterinarian Supervision. Prior to all studies, animals (e.g., mice and/or rats) were allowed to acclimate for a minimum of 3 days prior to use. The animals were maintained on a 12 hour light cycle in a temperature and humidity controlled room. A daily health check was performed as well as food and water check.

Injections were done subcutaneously (50-200 μL), intraperitoneally (up to 1000 μL) intravenously (50-200 μL), intramuscularly (50-μL), or intratumorally (50-μL), using a 26-30 gauge needle depending on the site of injection. Animals were under isoflurane anesthesia for all injections/implants.

For imaging, animals were put under anesthesia, injected intraperitoneally with D-Luciferin (15 mg/mL) at a dose of 10 μL per gram of body weight, and placed into the camera chamber and imaged for up to 30 minutes. Images were performed 15 minutes following substrate injection.

Intravenous administration. The delivery vehicle complexes disclosed herein are effective for in vivo administration of Fluc mRNA to BALB/c mice through multiple routes of administration. In general, delivery system complexes were administered at a dose of 0.5 mg/kg via a tail-vein injection, and the resulting bioluminescence was quantified after 6 hours. Organ-specific bioluminescence was quantified by sacrificing the treated animal, dissecting out the organs of interest, and separately quantifying the resulting bioluminescence

Local administration. In addition to IV administration, the delivery vehicle complexes described herein are also effective for local administration of mRNA through intratumoral (IT), subcutaneous (SC) or intramuscular (IM) routes of administration. For these examples, mRNA was administered at a dose of 0.1 mpk for intratumoral, or 0.01 mpk for subcutaneous and intramuscular administration, and the resulting bioluminescence was quantified after 6 hours.

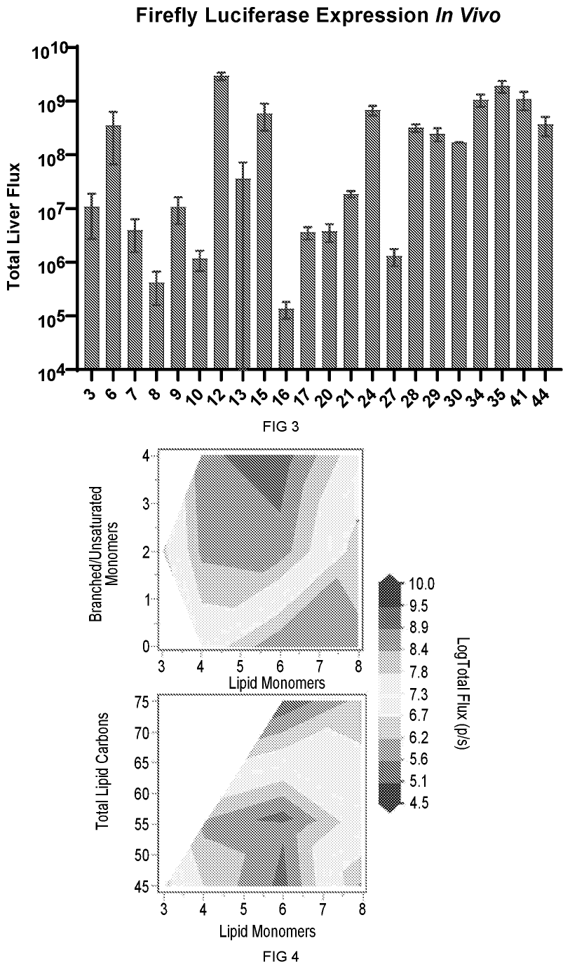

In one particular instance, to investigate how the peptoid lipid block impacts in vivo expression, delivery vehicles with diameter larger than 120 nm were excluded and remaining candidates were administered IV to BALB/c mice. Luminescence was measured after 6 hours and the resulting expression in liver, lung and spleen were quantified. Even without changing the cationic headgroup, luciferase expression spanned 4 orders of magnitude as a result of altering the lipid block in this series. Compounds 12, 41, 34, and 35 emerged as the top expressers from this screen (FIG. 3). Interestingly, these peptoids are all 6 monomers in length and contain the ethyl hexyl (Ehx) branched monomer. In vivo expression of all candidates was then fit to a multi-parameter model containing the 4 input parameters to understand the individual contributions of each factor to overall performance. This model suggests that expression has a very strong second order dependence on both the number of total lipid carbons, and the number of unstaturated or branched monomers, a strong dependence on if the peptoids use oleyl or Ehx as the unsaturated/branched monomer, and a modest dependence on the total number of lipid monomers. Contour plots of the impact of these factors on expression (all using Ehx as the branched/unsaturated monomer) are shown in FIG. 4.

Based on this model, the maximal predicted expression was achieved using peptoids containing 5-6 total lipid monomers, 55 total lipid carbons, and 2-3 ehx monomers as the non-linear lipid. Notably, 3 out of the 4 top performing peptoids (Compounds 41, 34, and 35) possessed attributes very closely aligned with this prediction. The fourth top candidate (Compound 12) had 3 out of 4 optimal attributes (lipid monomer number, non-linear monomers, and Ehx as the branched/unsaturated monomer), and the total number of lipid carbons was closely outside of this range (FIG. 4). This was encouraging and suggested that the parameterization scheme utilized was fairly predictive of the activity of peptoid-based ionizable lipids. With confidence in the lipid block optimization process, these top 4 candidates were advanced into additional experiments to evaluate their in vivo production of secreted proteins.

For imaging studies, female BALB/c mice (6-8 weeks old) were obtained and acclimatized for a minimum of 3 days prior to experiments. The animals were maintained on a 12 hour light cycle in a temperature and humidity controlled room. A daily health check was performed as well as food and water check. For dosing, mice were injected with 2.5 μg fLuc mRNA-containing delivery vehicles intravenously in 100 μL total injection volume. At 6 hours post-injection, mice were anesthetized with isoflurane, and injected intraperitoneally with 30 mg/mL D-luciferin at a dose of 10 μL per gram bodyweight intraperitoneally. Imaging was done 10 minutes post D-luciferin injection using an IVIS® Spectrum Imaging System. Immediately after in vivo imaging, mice were terminally bled by cardiac puncture and euthanized. The organs of interest were harvested, placed in black 24-well plates, and imaged. Living Image® software was used to quantify the total photon flux in regions of interest. For secreted protein studies, mice were injected with aRSV mRNA-containing delivery vehicles intravenously in 100 μL total injection volume. Mice were euthanized after 24 hours, with serum collection at 6 and 24 hours post-injection. Serum was used to quantify secreted aRSV levels using ELISA following the manufacturer's recommendation for IgG using a standard curve generated from aRSV protein.

CryoTEM was also performed. 3 μL of approximately 1E14 particles/mL was applied to a clean grid (holey carbon film on 400 mesh coper), blotted with filter paper, then repeated once more before immediate vitrification in liquid ethane. Micrographs were acquired using a cryo-transmission electron microscope (Cryo-TEM) operated at 200 kV and equipped with a direct electron detector and software for automated data collection. High magnification images were acquired at nominal magnifications of 73,000× (0.200 nm/pixel) and 28,000× (0.524 nm/pixel). The images were acquired at a nominal underfocus of −5.5 μm to −3.5 μm and electron doses of ˜10-25 e-/Å2.

### Example 6—Efficacy and Toxicology Studies

Compounds of Formula (I) were tested in mouse efficacy and rat toxicology studies. Briefly, Sprague-Dawley rats were treated with a control mRNA formulated in e.g., F2 at 0.03 or 0.3 mg/kg or PBS vehicle. Injections were done 4 times over the course of 13 days and were administered intramuscularly into the hind limb. Whole blood was taken for hematology, and serum taken for clinical chemistry and cytokine analysis at 6 hours post the first dose and final dose, and 2 weeks post the final dose. Additionally, two of the animals were sacrificed at 6 hours post the final dose and 2 weeks post final dose and a gross necropsy was performed. Tissue samples were retained and histopathology run on select organs.

### Example 7—Protein Expression Studies

To understand how the delivery vehicles disclosed herein (e.g., delivery vehicles comprising compounds of Formula (I)) performed as systemic delivery vehicles, the top four candidates for firefly luciferase expression from the optimized DOE model (Compounds 12, 41, 34, and 35) were evaluated for their ability to produce anti-RSV antibody in BALB/c mice. Anti-RSV was chosen as a model target due to the availability of therapeutic benchmarking information and sequence availability. Delivery vehicles comprising aRSV were formulated from two mRNA molecules encoding the heavy and light chain at a 2:1 mass ratio. All delivery vehicles were below 120 nm in size with encapsulation greater than 85% (Table 7).

TABLE 7
Characterization data of delivery vehicles containing
Compounds 12, 41, 34, and 35 in Formulation D22.

| Delivery | | | | encap |
|---|---|---|---|---|
| Vehicle | Diameter | PDI | Encap | stdev |
| 34-D22 | 71.8 | 0.087 | 88.5 | 0.3 |
| 35-D22 | 71.7 | 0.09 | 92.3 | 0.6 |
| 12-D22 | 67.6 | 0.065 | 95.8 | 0.9 |
| 41-D22 | 80.2 | 0.078 | 96.1 | 0.2 |

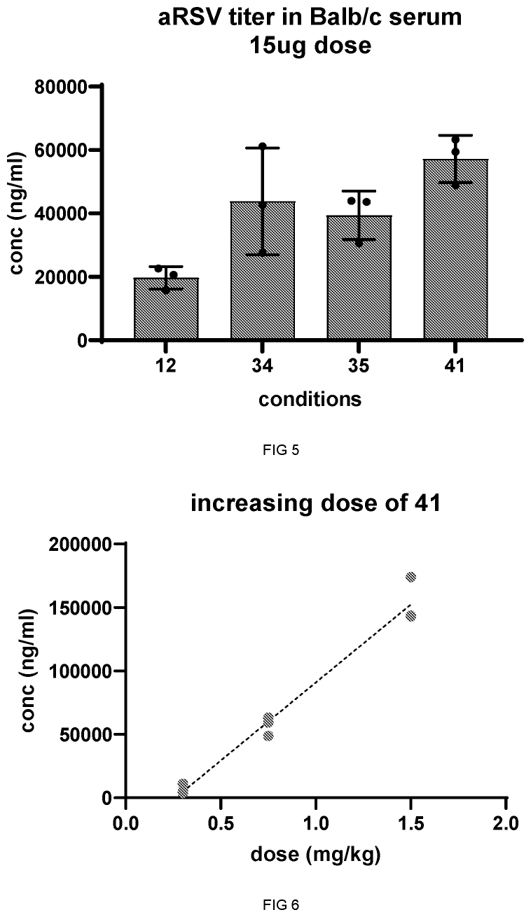

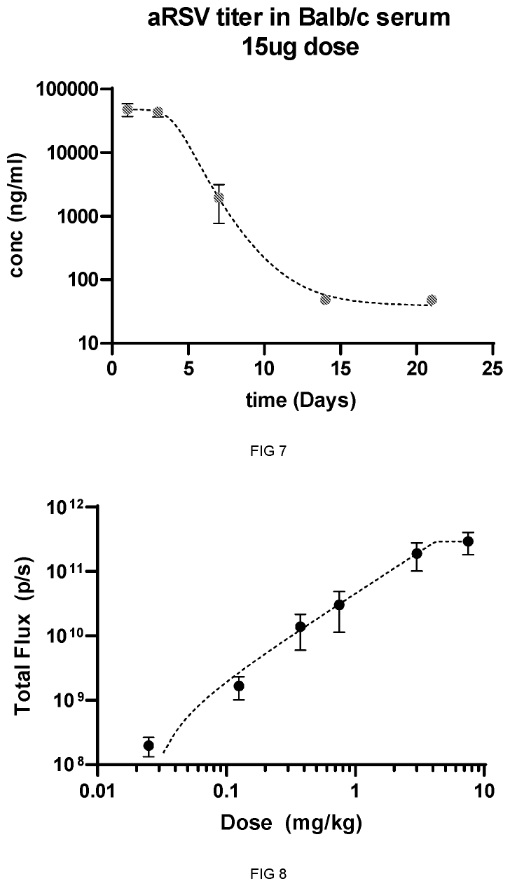

Delivery vehicles were dosed intravenously in BALB/c mice at a total dose of 0.75 mg/kg mRNA and serum levels of aRSV were compared after 24 hours by an IgG ELISA. Compounds 34 and 41 had the highest levels of secreted protein expression (FIG. 5). Interestingly, Compound 12, which had the highest overall fLuc expression, had the lowest aRSV expression of the top 4 candidates, indicating that there was not a perfect correlation between expression of intracellular proteins such as fLuc, and secreted ones like aRSV. Additional PK and dose characterization was carried out on Compound 41 formulated in Formulation D22, which showed the highest overall aRSV titers (FIG. 6). With this candidate, aRSV titers were observed in the serum past 5 days, and serum titers showed a generally linear dependence on injected dose at 0.3, 0.75, and 1.5 mg/kg (FIG. 7). The gross tolerability of the Compound 41-D22 formulation was further probed using the fLuc cargo mRNA by escalating doses from 0.3 mg/kg to 7.5 mg/kg and no immediate tolerability concerns were observed across this wide dose range (FIG. 8).

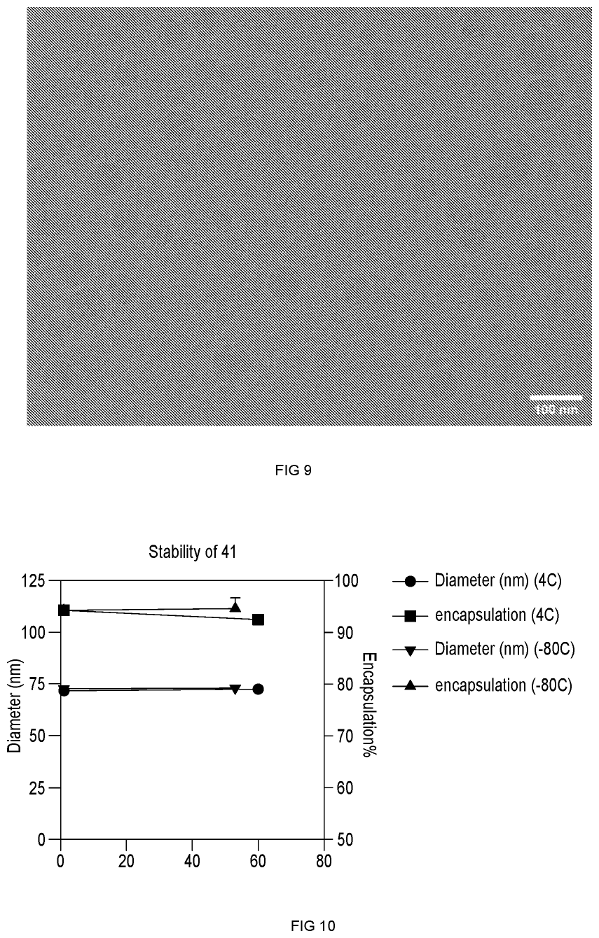

The particle structure of the Compound 41-D22 formulation was characterized with cryo-TEM, and its stability was monitored over time. The majority of Compound 41-D22 particles show a dense core morphology without bleb features observed (FIG. 9). Stability was monitored at 4° C. or −80° C. storage for over 1 month with no growth or change in encapsulation observed (FIG. 10). Together, the spherical dense particle morphology in addition to the 4° C. and frozen stability support the practical use of the delivery vehicles disclosed herein for therapeutic use.

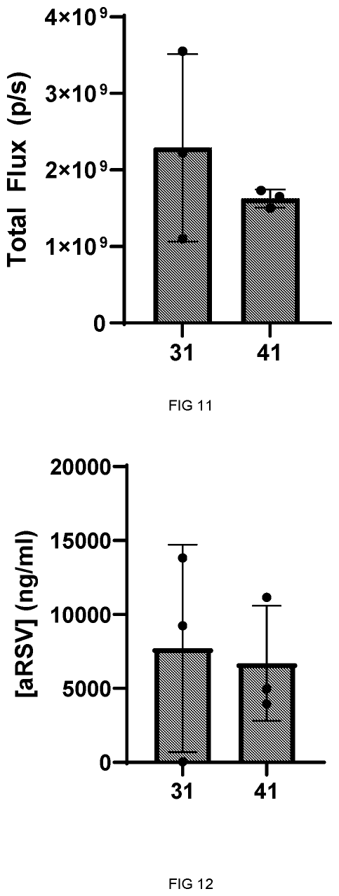

The impact of monomer lipid order on the delivery vehicle's performance was also explored. To accomplish this, Compound 31 was synthesized which subtly alters the order of C12 and Ehx monomers in the lipid block compared to the monomers used in Compound 41. Physical characteristics such as size, encapsulation, and hemolysis were all nearly identical between these two isomers (Table 5). In vivo, both luciferase expression and secreted protein expression were found to be within error for the two order variations, suggesting that monomer composition, rather than order, drives differences between peptoids (FIGS. 11 and 12). This further validates the screening funnel employed and future peptoid variations could look at order variations as secondary factor only.

The preceding disclosure shows that peptoid based lipid nanoparticles provide a highly-tunable platform for optimizing mRNA delivery for both intracellular and secreted protein expression. Through a broad headgroup screen, headgroup tuning was found to change the expression of delivery vehicles among the major organs including lung, spleen, and liver. From this initial screen, the 1,3 diol head group with highest liver selectivity was chosen for lipid block optimization. The aforementioned parameterization strategy for the lipid block identified ideal combinations of number of lipid monomers, total number of lipid carbons, number of branched or unsaturated monomers, and whether 2-ethylhexyl or oleyl monomers were used. Optimized delivery vehicles showed liver selectivity above 90% and were used to express sustained titers of aRSV in mouse serum validating the platform for systemic delivery with no tolerability concerns observed. While further studies in higher species are ongoing to explore tolerability, these data support delivery vehicles comprising the compounds disclosed herein (e.g., compounds of Formula (I) as a demonstrative platform for mRNA therapeutic delivery which can be optimized for a given therapeutic target.

It should be appreciated that all combinations of the foregoing concepts and implementations and additional concepts and implementations discussed in greater detail below are contemplated as being part of the inventive subject matter disclosed herein, and may be employed in any suitable combination to achieve the benefits as described here. In particular, all combinations of claimed subject matter appearing at the end of this disclosure are contemplated as being part of the inventive subject matter disclosed herein. The foregoing description is given for clearness of understanding only, and no unnecessary limitations should be understood therefrom, as modifications within the scope of the disclosure may be apparent to those having ordinary skill in the art.

The terms “substantially” and “about” used throughout this Specification are used to describe and account for small fluctuations. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The disclosure illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular implementations, a person of ordinary skill in the art will readily appreciate that other ways of performing the acts associated with the methods may be used. For example, the order of various steps may be changed without departing from the scope or spirit of the method, unless described otherwise. In addition, some of the individual steps can be combined, omitted, or further subdivided into additional steps.

All patents, publications and references cited herein are hereby fully incorporated by reference. In case of conflict between the present disclosure and incorporated patents, publications and references, the present disclosure should control.

1. A compound having a structure of Formula (I): 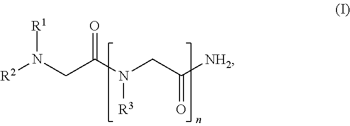
wherein
n is 1, 2, 3, 4, 5, 6, 7, 8, 9, or 10;
R¹ is H or C₂₋₅alkyl optionally substituted with 1-3 OH;
R² is C₂₋₅alkylene-OH substituted with 1-3 additional OH; and
each R³ independently is C₆₋₂₄alkyl or C₆₋₂₄alkenyl.

2. The compound of claim 1, wherein n is 3, 4, 6, 8, or 9.

3.-6. (canceled)

7. The compound of claim 1, wherein R¹ is H.

8. The compound of claim 1, wherein R² is C₂₋₅alkylene-OH substituted with 1 additional OH.

9. The compound of claim 1, wherein R² is C₂₋₅alkylene-OH substituted with 2 or 3 additional OH.

10. (canceled)

11. The compound of claim 1, wherein each R³ independently is C₆₋₁₈alkyl or C₆₋₁₈alkenyl.

12. (canceled)

13. The compound of claim 1, wherein each R³ independently is selected from the group consisting of

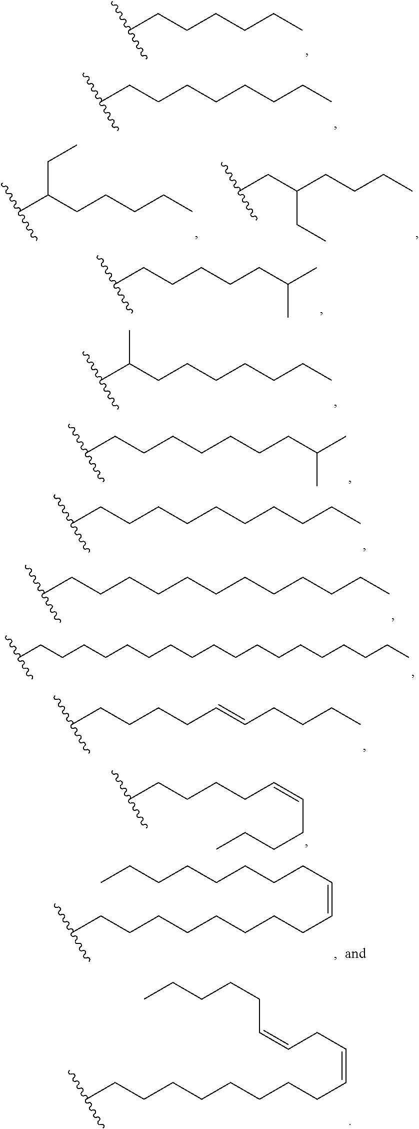

14. (canceled)

15. The compound of claim 1, wherein at least one R³ is selected from the group consisting of

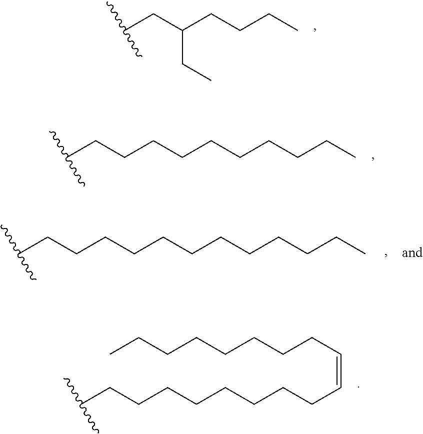

16. (canceled)

17. The compound of claim 1, having a structure as recited in Table 1:
TABLE 1

| Cmpd | Structure |
|---|---|
| 1 | 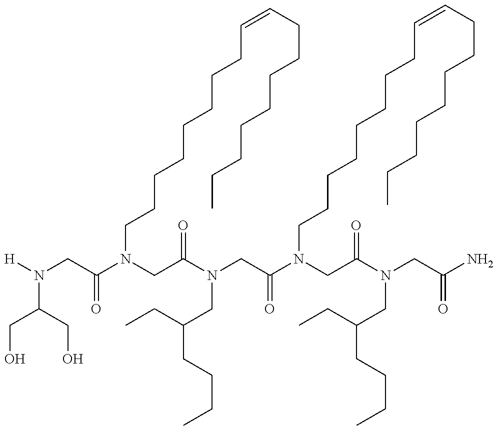 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNC(CO)CO] |
| 2 | 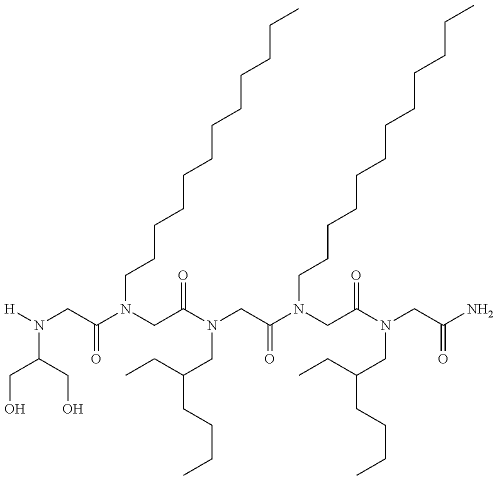 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 3 | 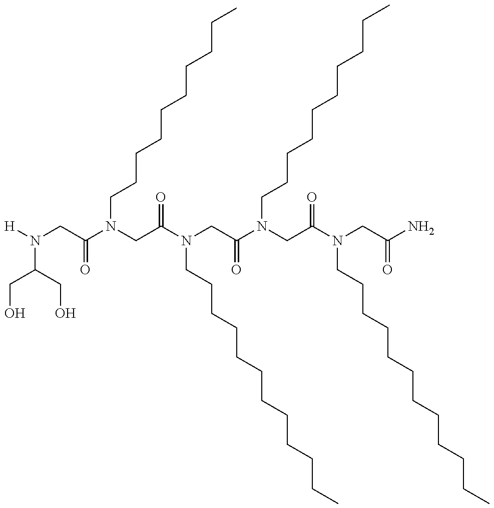 [SMILES: CCCCCCCCCCCCN(CC(N)=O)C(=O)CN(CCCCCCCCCC)C(=O)CN(CCCCCCCCCCCC)C(=O)CN(CCCCCCCCCC)C(=O)CNC(CO)CO] |
| 4 | 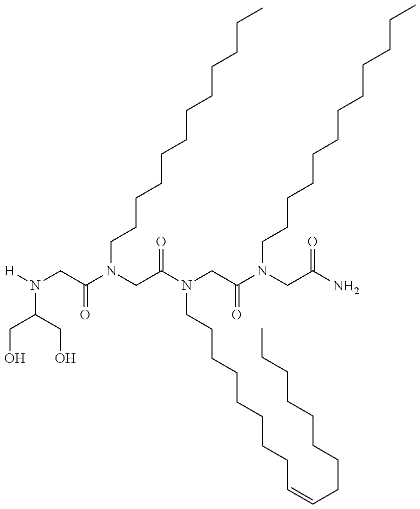 [SMILES: CCCCCCCC/C=C\CCCCCCCCN(CC(=O)N(CCCCCCCCCCCC)CC(N)=O)C(=O)CN(CCCCCCCCCCCC)C(=O)CNC(CO)CO] |
| 5 | 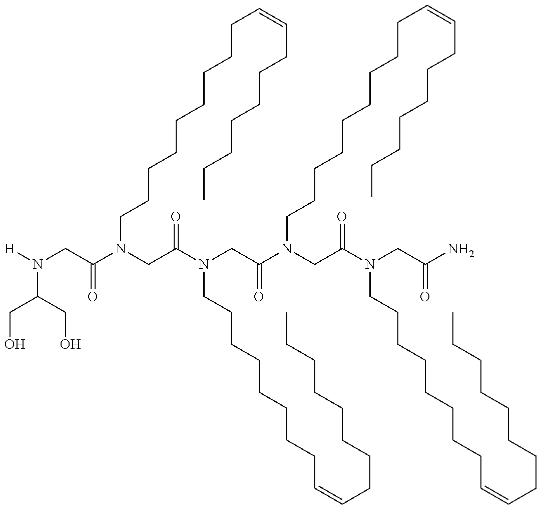 [SMILES: CCCCCCC/C=C\CCCCCCCCCN(CC(=O)N(CCCCCCCC/C=C\CCCCCCCC)CC(=O)N(CCCCCCCCC/C=C\CCCCCCC)CC(=O)N(CCCCCCCC/C=C\CCCCCCCC)CC(N)=O)C(=O)CNC(CO)CO] |
| 6 | 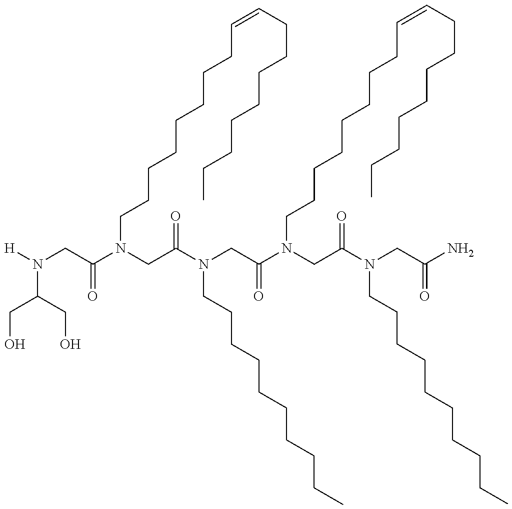 [SMILES: CCCCCCCC/C=C\CCCCCCCCN(CC(=O)N(CCCCCCCCCC)CC(=O)N(CCCCCCCC/C=C\CCCCCCCC)CC(=O)N(CCCCCCCCCC)CC(N)=O)C(=O)CNC(CO)CO] |
| 7 | 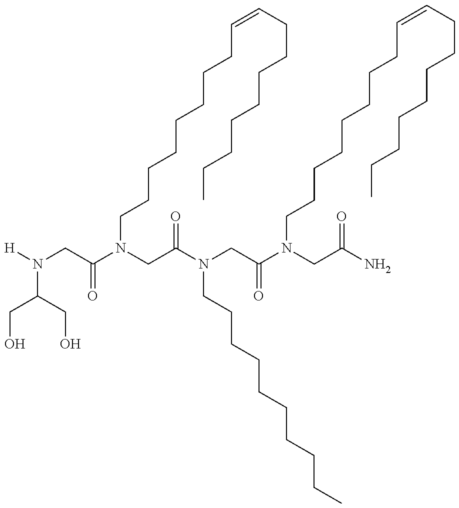 [SMILES: CCCCCCCC/C=C\CCCCCCCCN(CC(N)=O)C(=O)CN(CCCCCCCCCC)C(=O)CN(CCCCCCCC/C=C\CCCCCCCC)C(=O)CNC(CO)CO] |
| 8 | 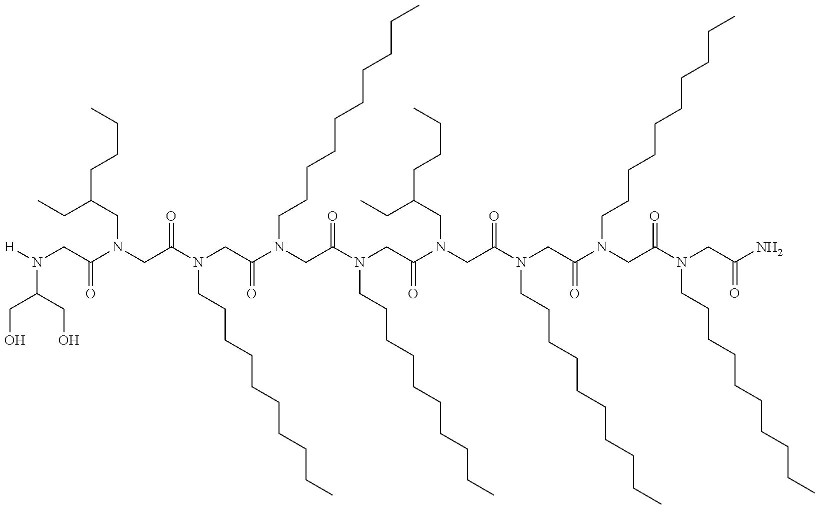 [SMILES: CCCCCCCCCCN(CC(N)=O)C(=O)CN(CCCCCCCCCC)C(=O)CN(CCCCCCCCCC)C(=O)CN(CC(CC)CCCC)C(=O)CN(CCCCCCCCCC)C(=O)CN(CCCCCCCCCC)C(=O)CN(CCCCCCCCCC)C(=O)CN(CC(CC)CCCC)C(=O)CNC(CO)CO] |
| 9 | 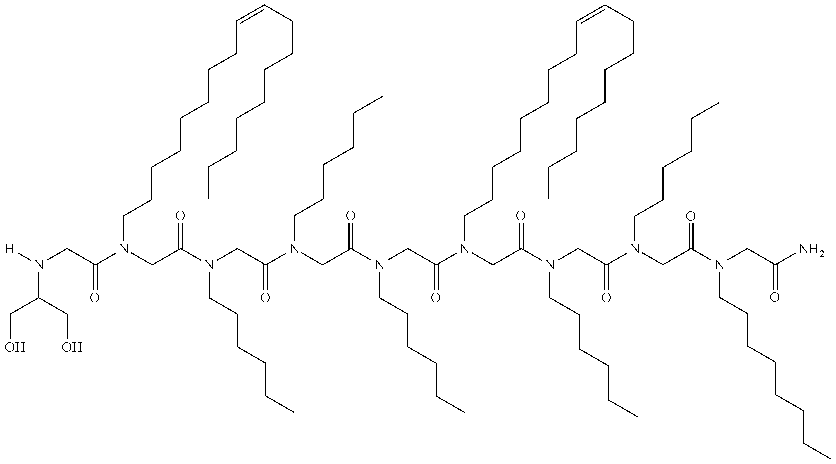 [SMILES: CCCCCCCC/C=C\CCCCCCCCN(CC(=O)N(CCCCCC)CC(=O)N(CCCCCC)CC(=O)N(CCCCCC)CC(=O)N(CCCCCCCC/C=C\CCCCCCCC)CC(=O)N(CCCCCC)CC(=O)N(CCCCCC)CC(=O)N(CCCCCCCC)CC(N)=O)C(=O)CNC(CO)CO] |
| 10 | 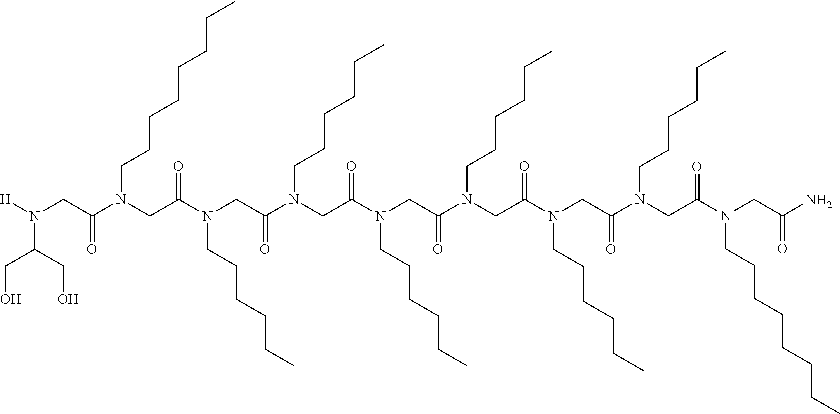 [SMILES: CCCCCCCCN(CC(N)=O)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CN(CCCCCCCC)C(=O)CNC(CO)CO] |
| 11 | 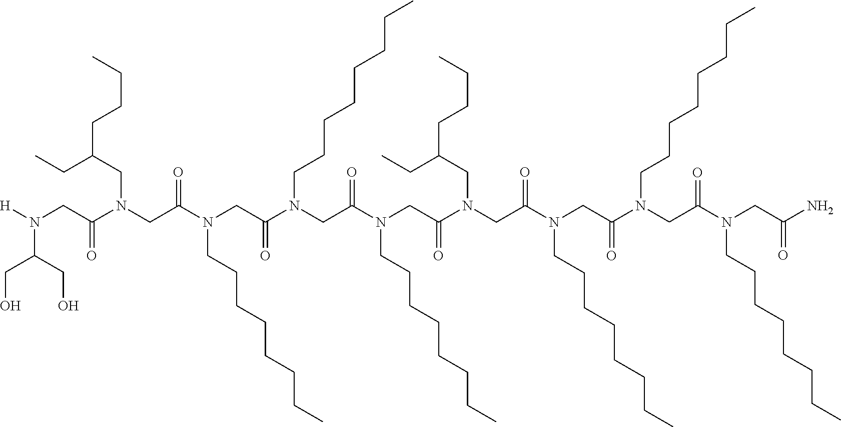 [SMILES: CCCCCCCCN(CC(N)=O)C(=O)CN(CCCCCCCC)C(=O)CN(CCCCCCCC)C(=O)CN(CC(CC)CCCC)C(=O)CN(CCCCCCCC)C(=O)CN(CCCCCCCC)C(=O)CN(CCCCCCCC)C(=O)CN(CC(CC)CCCC)C(=O)CNC(CO)CO] |
| 12 | 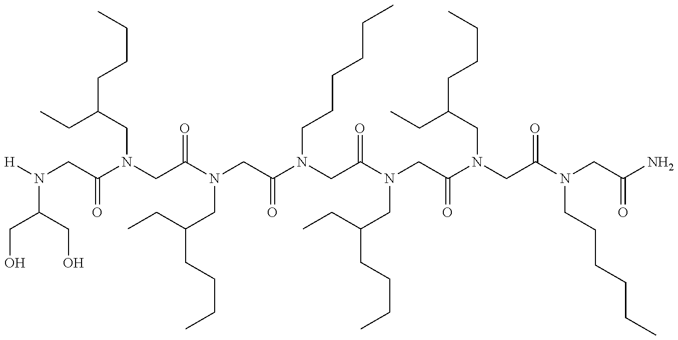 [SMILES: CCCCCCN(CC(N)=O)C(=O)CN(CC(CC)CCCC)C(=O)CN(CC(CC)CCCC)C(=O)CN(CCCCCC)C(=O)CN(CC(CC)CCCC)C(=O)CN(CC(CC)CCCC)C(=O)CNC(CO)CO] |
| 13 | 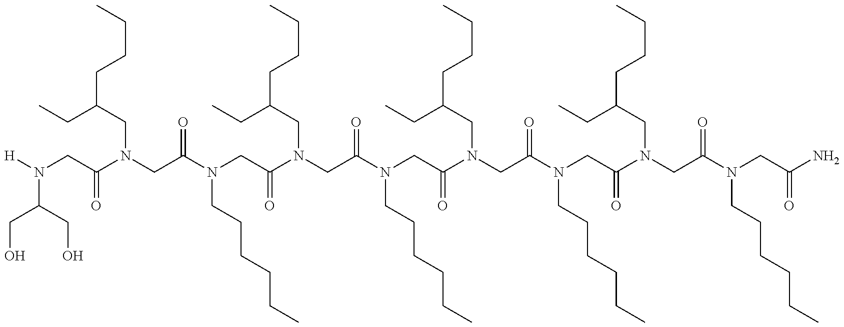 [SMILES: CCCCCCN(CC(N)=O)C(=O)CN(CC(CC)CCCC)C(=O)CN(CCCCCC)C(=O)CN(CC(CC)CCCC)C(=O)CN(CCCCCC)C(=O)CN(CC(CC)CCCC)C(=O)CN(CCCCCC)C(=O)CN(CC(CC)CCCC)C(=O)CNC(CO)CO] |
| 14 | 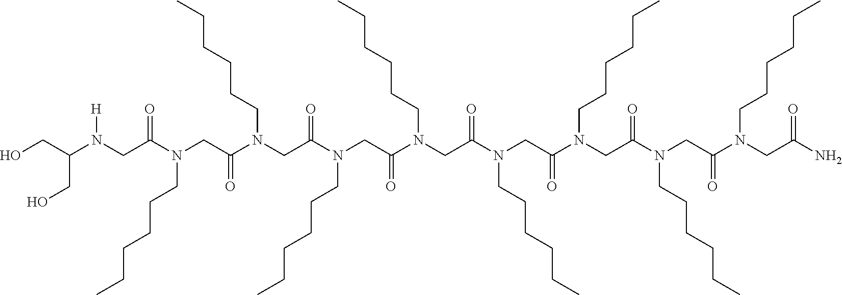 [SMILES: CCCCCCN(CC(N)=O)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CN(CCCCCC)C(=O)CNC(CO)CO] |
| 15 | 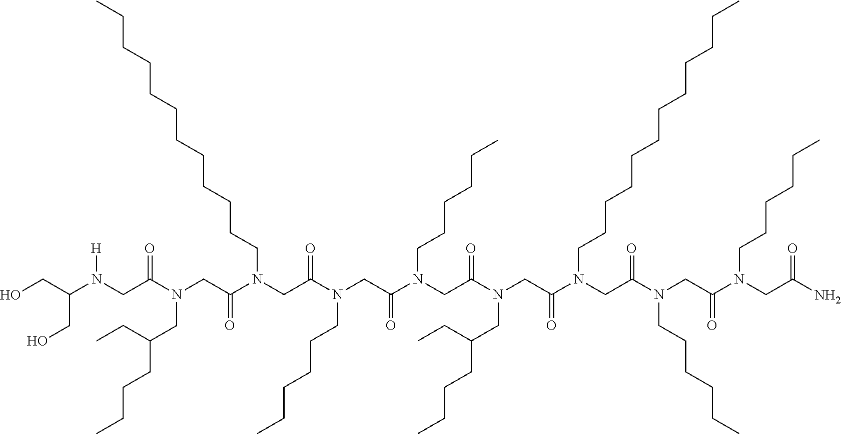 [SMILES: CCCCCCCCCCCCN(CC(=O)N(CCCCCC)CC(=O)N(CCCCCC)CC(=O)N(CC(=O)N(CCCCCCCCCCCC)CC(=O)N(CCCCCC)CC(=O)N(CCCCCC)CC(N)=O)CC(CC)CCCC)C(=O)CN(CC(CC)CCCC)C(=O)CNC(CO)CO] |
| 16 | 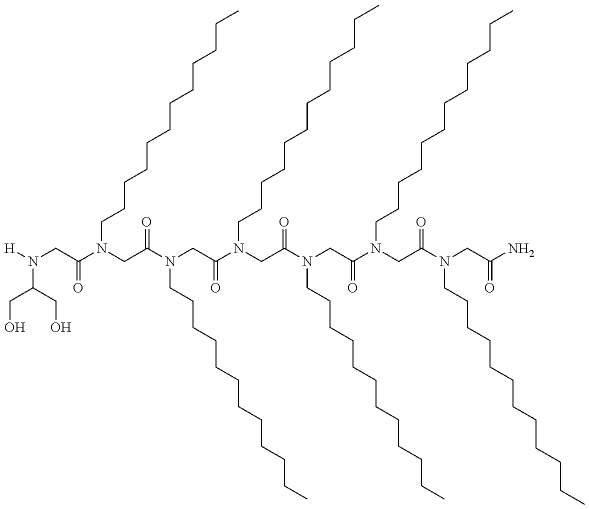 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNC(CO)CO] |
| 17 | 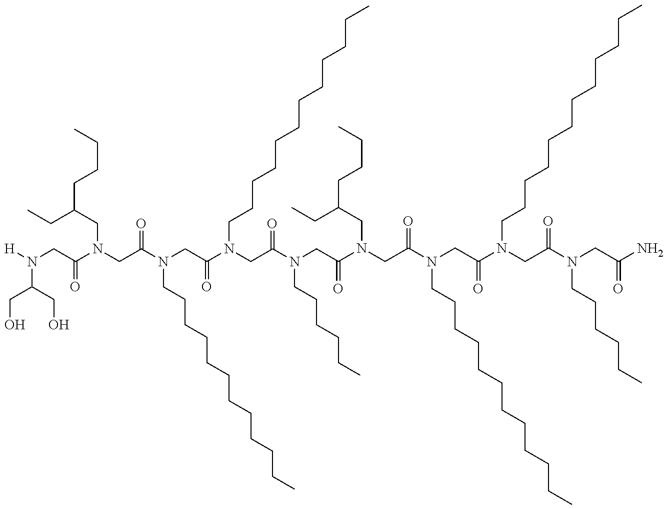 [SMILES: CCCCCCCCCCCCN(CC(=O)N(CCCCCC)CC(N)=O)C(=O)CN(CCCCCCCCCCCC)C(=O)CN(CC(CC)CCCC)C(=O)CN(CCCCCC)C(=O)CN(CCCCCCCCCCCC)C(=O)CN(CCCCCCCCCCCC)C(=O)CN(CC(CC)CCCC)C(=O)CNC(CO)CO] |
| 18 | 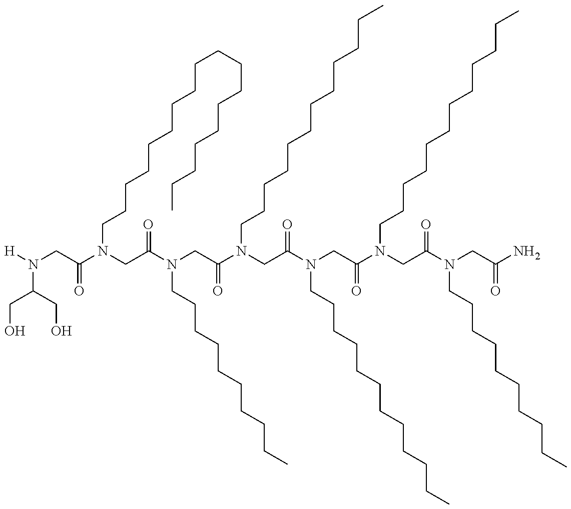 [SMILES: CCCCCCCCCCCCCCCCCCN(CC(=O)N(CCCCCCCCCC)CC(=O)N(CCCCCCCCCCCC)CC(=O)N(CCCCCCCCCCCC)CC(=O)N(CCCCCCCCCCCC)CC(=O)N(CCCCCCCCCC)CC(N)=O)C(=O)CNC(CO)CO] |
| 19 | 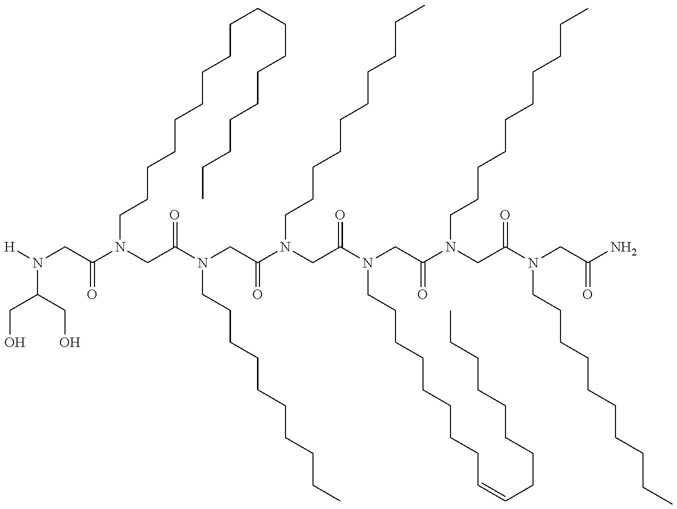 [SMILES: CCCCCCCC/C=C\CCCCCCCCN(CC(=O)N(CCCCCCCCCC)CC(=O)N(CCCCCCCCCC)CC(N)=O)C(=O)CN(CCCCCCCCCC)C(=O)CN(CCCCCCCCCC)C(=O)CN(CCCCCCCCCCCCCCCCCC)C(=O)CNC(CO)CO] |
| 20 | 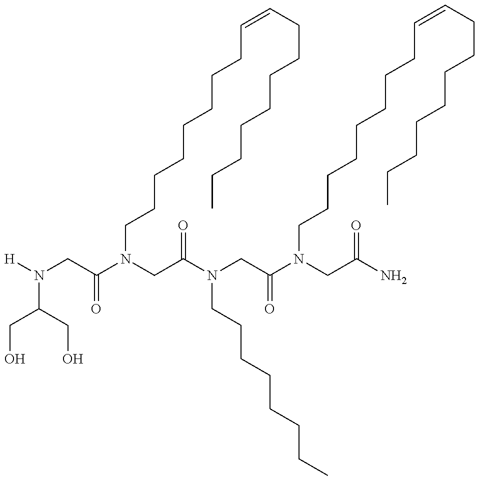 [SMILES: CCCCCCCC/C=C\CCCCCCCCN(CC(N)=O)C(=O)CN(CCCCCCCC)C(=O)CN(CCCCCCCC/C=C\CCCCCCCC)C(=O)CNC(CO)CO] |
| 21 | 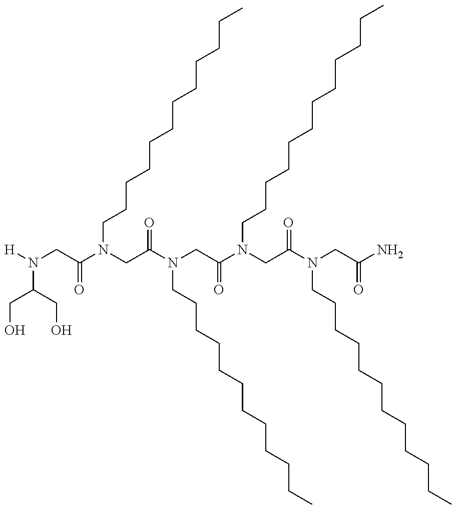 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNC(CO)CO] |
| 22 | 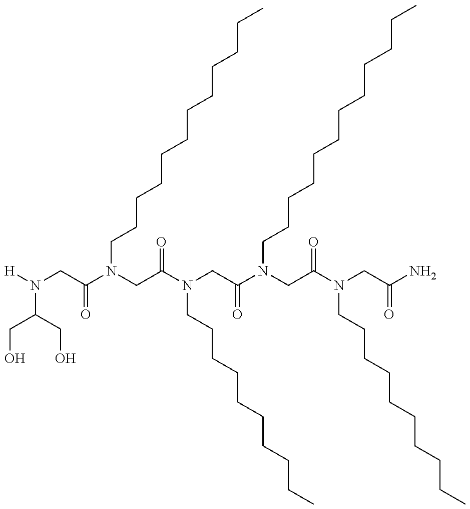 [SMILES: CCCCCCCCCCCCN(CC(=O)N(CCCCCCCCCC)CC(=O)N(CCCCCCCCCCCC)CC(=O)N(CCCCCCCCCC)CC(N)=O)C(=O)CNC(CO)CO] |
| 23 | 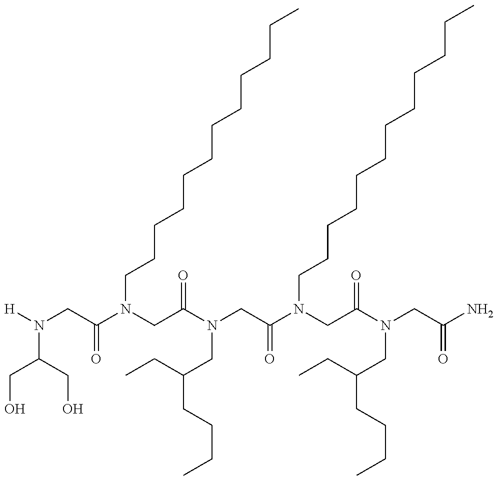 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 24 | 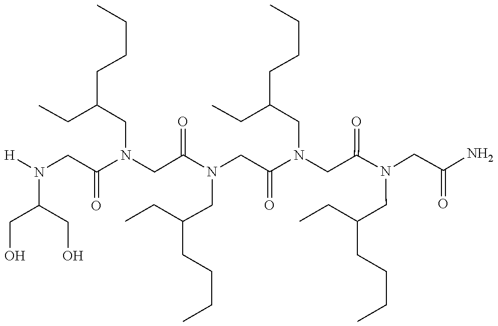 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CNC(CO)CO] |
| 25 | 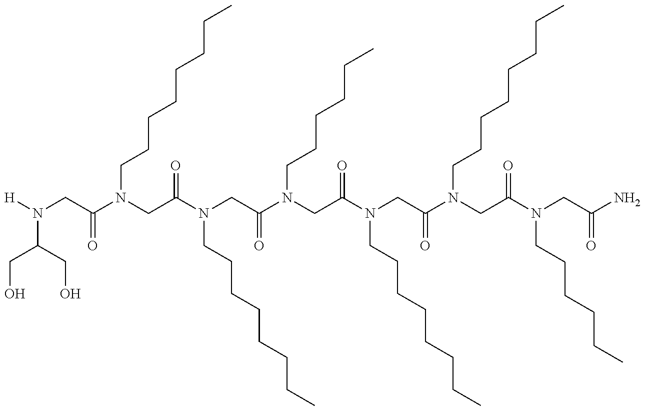 [SMILES: CCCCCCCCN(CC(=O)N(CCCCCCCC)CC(=O)N(CCCCCC)CC(=O)N(CCCCCCCC)CC(=O)N(CCCCCCCC)CC(=O)N(CCCCCC)CC(N)=O)C(=O)CNC(CO)CO] |
| 26 | 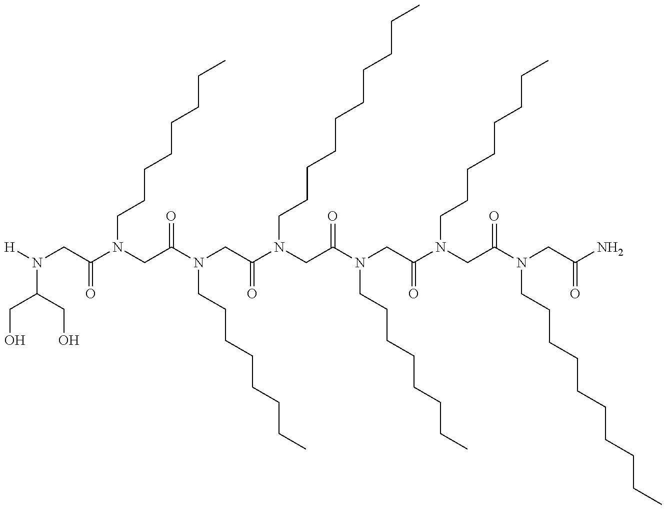 [SMILES: CCCCCCCCCCN(CC(N)=O)C(=O)CN(CCCCCCCC)C(=O)CN(CCCCCCCC)C(=O)CN(CCCCCCCCCC)C(=O)CN(CCCCCCCC)C(=O)CN(CCCCCCCC)C(=O)CNC(CO)CO] |
| 27 | 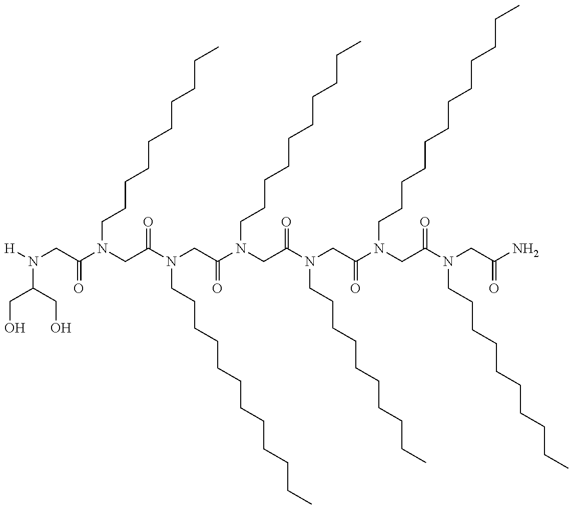 [SMILES: CCCCCCCCCCCCN(CC(=O)N(CCCCCCCCCC)CC(N)=O)C(=O)CN(CCCCCCCCCC)C(=O)CN(CCCCCCCCCC)C(=O)CN(CCCCCCCCCCCC)C(=O)CN(CCCCCCCCCC)C(=O)CNC(CO)CO] |
| 28 | 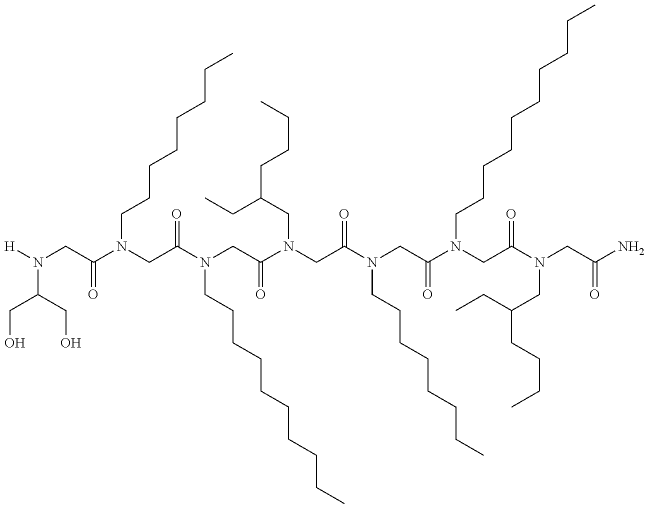 [SMILES: CCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCC)C(=O)CN(CCCCCCCC)C(=O)CNC(CO)CO] |
| 29 | 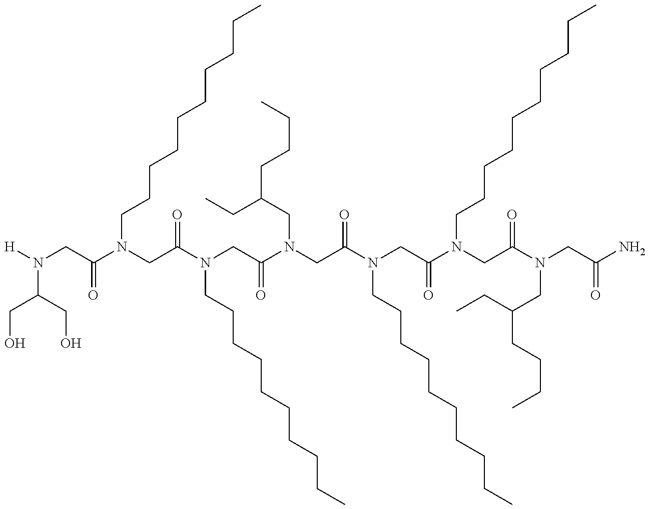 [SMILES: CCCCCCCCCCN(CC(=O)N(CCCCCCCCCC)CC(=O)N(CC(=O)N(CCCCCCCCCC)CC(=O)N(CCCCCCCCCC)CC(=O)N(CC(N)=O)CC(CC)CCCC)CC(CC)CCCC)C(=O)CNC(CO)CO] |
| 30 | 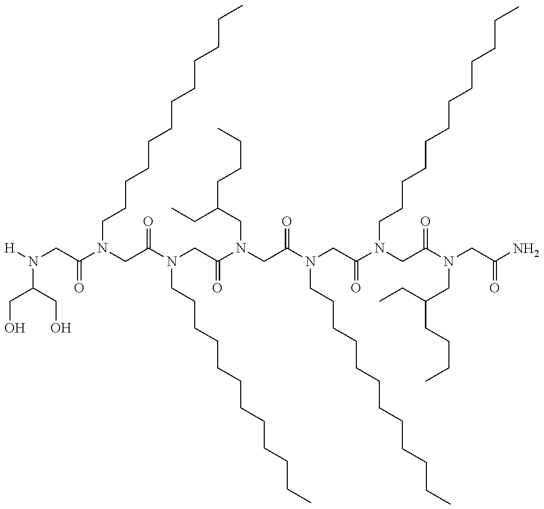 [SMILES: CCCCCCCCCCCCN(CC(=O)N(CCCCCCCCCCCC)CC(=O)N(CC(=O)N(CCCCCCCCCCCC)CC(=O)N(CCCCCCCCCCCC)CC(=O)N(CC(N)=O)CC(CC)CCCC)CC(CC)CCCC)C(=O)CNC(CO)CO] |
| 31 | 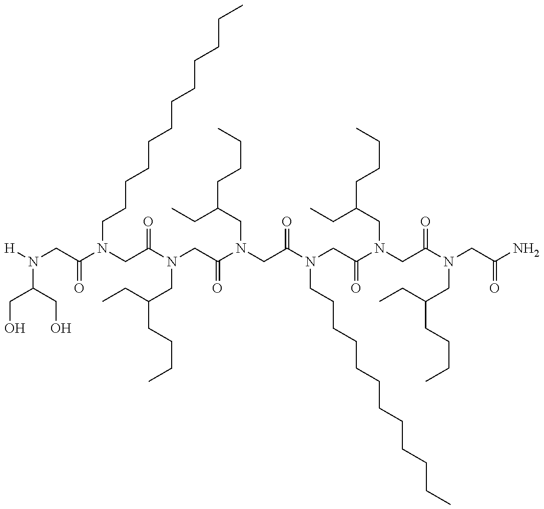 [SMILES: CCCCCCCCCCCCN(CC(=O)N(CC(=O)N(CC(=O)N(CCCCCCCCCCCC)CC(=O)N(CC(=O)N(CC(N)=O)CC(CC)CCCC)CC(CC)CCCC)CC(CC)CCCC)CC(CC)CCCC)C(=O)CNC(CO)CO] |
| 32 | 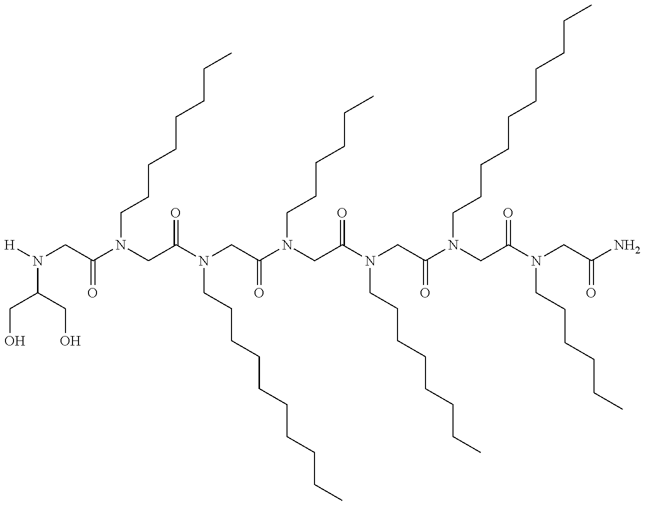 [SMILES: CCCCCCCCCCN(CC(=O)N(CCCCCC)CC(N)=O)C(=O)CN(CCCCCCCC)C(=O)CN(CCCCCC)C(=O)CN(CCCCCCCCCC)C(=O)CN(CCCCCCCC)C(=O)CNC(CO)CO] |
| 33 | 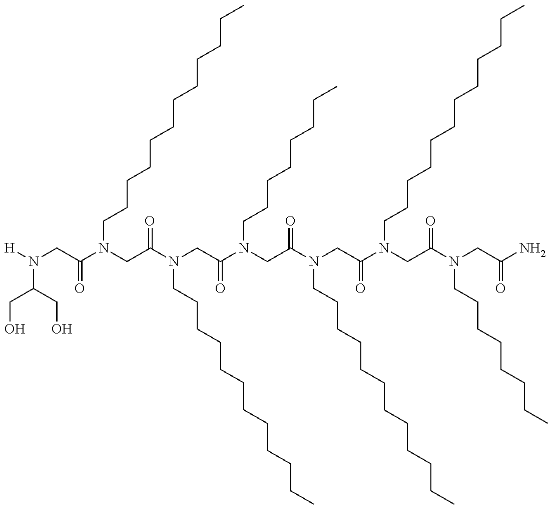 [SMILES: CCCCCCCCCCCCN(CC(=O)N(CCCCCCCCCCCC)CC(=O)N(CCCCCCCC)CC(=O)N(CCCCCCCCCCCC)CC(=O)N(CCCCCCCCCCCC)CC(=O)N(CCCCCCCC)CC(N)=O)C(=O)CNC(CO)CO] |
| 34 | 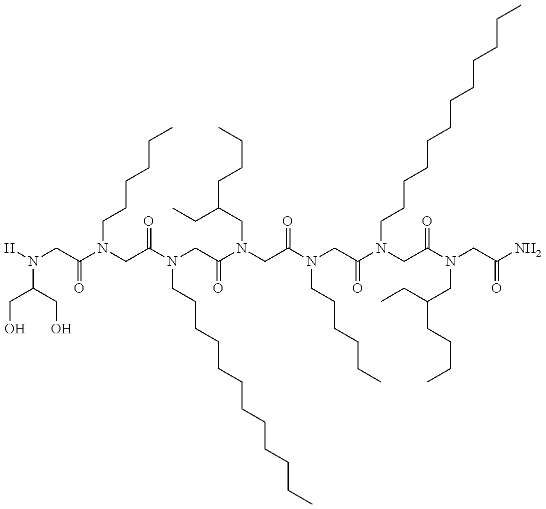 [SMILES: CCCCCCCCCCCCN(CC(=O)N(CC(N)=O)CC(CC)CCCC)C(=O)CN(CCCCCC)C(=O)CN(CC(CC)CCCC)C(=O)CN(CCCCCCCCCCCC)C(=O)CN(CCCCCC)C(=O)CNC(CO)CO] |
| 35 | 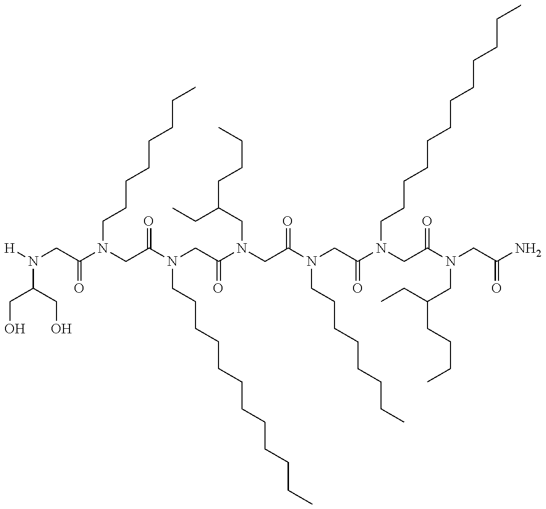 [SMILES: CCCCCCCCCCCCN(CC(=O)N(CC(N)=O)CC(CC)CCCC)C(=O)CN(CCCCCCCC)C(=O)CN(CC(CC)CCCC)C(=O)CN(CCCCCCCCCCCC)C(=O)CN(CCCCCCCC)C(=O)CNC(CO)CO] |
| 36 | 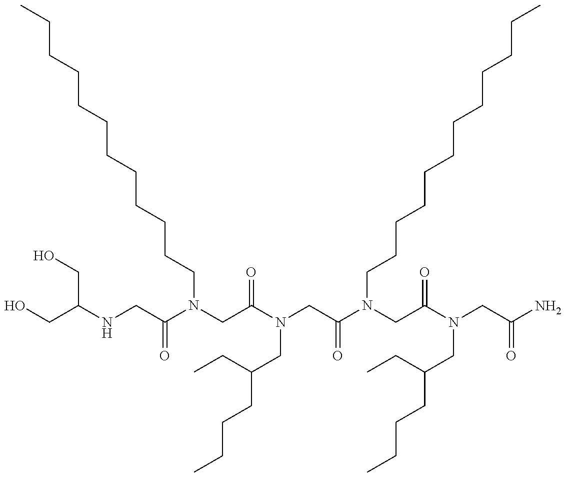 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNC(CO)CO] |
| 37 | 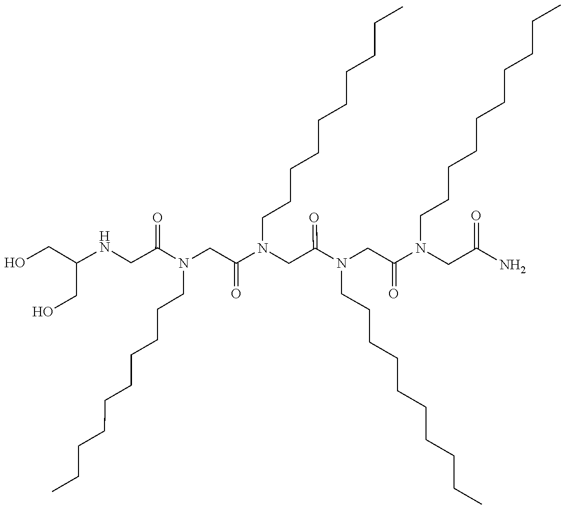 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNC(CO)CO] |
| 40 | 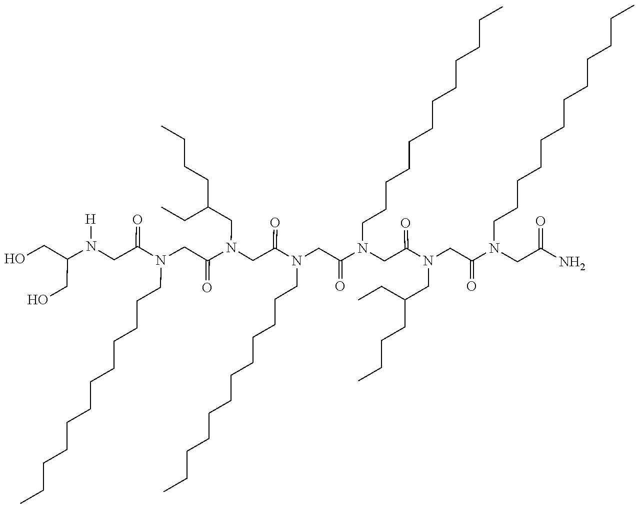 [SMILES: CCCCCCCCCCCCN(CC(N)=O)C(=O)CN(CC(CC)CCCC)C(=O)CN(CCCCCCCCCCCC)C(=O)CN(CCCCCCCCCCCC)C(=O)CN(CC(CC)CCCC)C(=O)CN(CCCCCCCCCCCC)C(=O)CNC(CO)CO] |
| 41 | 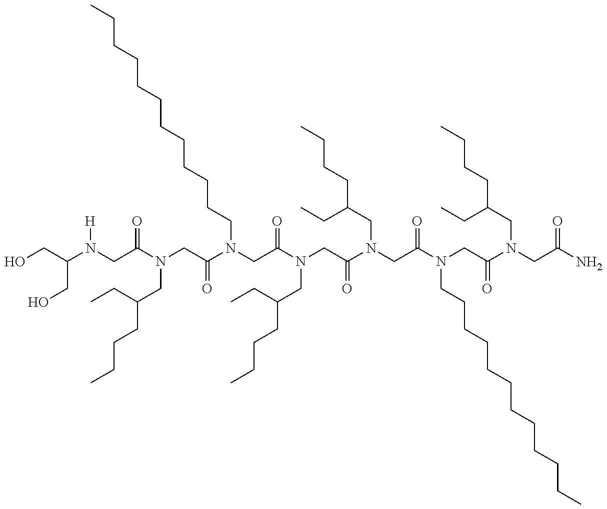 [SMILES: CCCCCCCCCCCCN(CC(=O)N(CC(=O)N(CC(=O)N(CCCCCCCCCCCC)CC(=O)N(CC(N)=O)CC(CC)CCCC)CC(CC)CCCC)CC(CC)CCCC)C(=O)CN(CC(CC)CCCC)C(=O)CNC(CO)CO] |
| 42 | 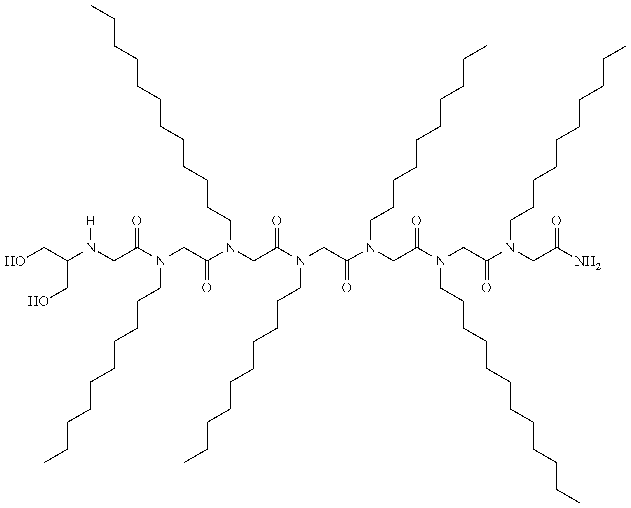 [SMILES: CCCCCCCCCCCCN(CC(=O)N(CCCCCCCCCC)CC(N)=O)C(=O)CN(CCCCCCCCCC)C(=O)CN(CCCCCCCCCC)C(=O)CN(CCCCCCCCCCCC)C(=O)CN(CCCCCCCCCC)C(=O)CNC(CO)CO] |
| 43 | 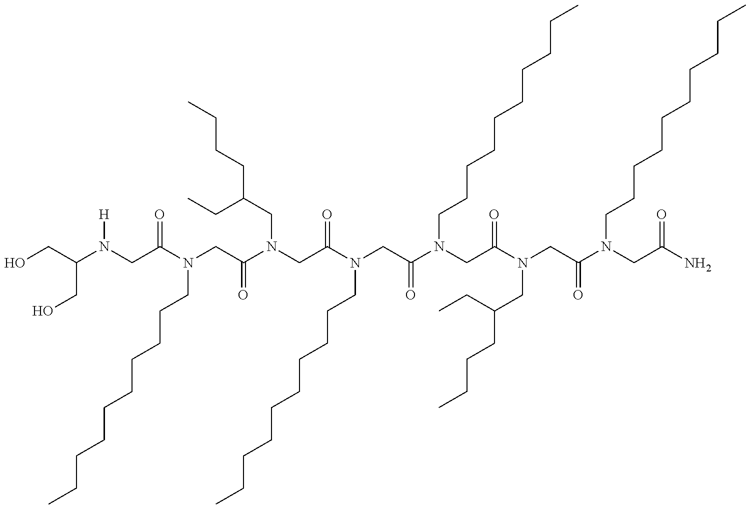 [SMILES: CCCCCCCCCCN(CC(N)=O)C(=O)CN(CC(CC)CCCC)C(=O)CN(CCCCCCCCCC)C(=O)CN(CCCCCCCCCC)C(=O)CN(CC(CC)CCCC)C(=O)CN(CCCCCCCCCC)C(=O)CNC(CO)CO] |
| 44 | 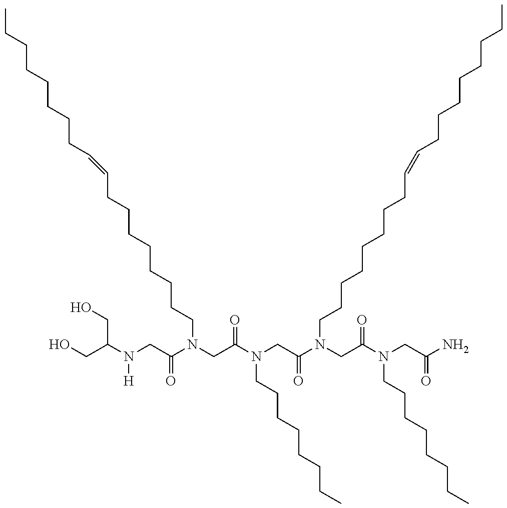 [SMILES: CCCCCCCC/C=C\CCCCCCCCN(CC(=O)N(CCCCCCCC)CC(=O)N(CCCCCCCC/C=C\CCCCCCCC)CC(=O)N(CCCCCCCC)CC(N)=O)C(=O)CNC(CO)CO] |
| 45 | 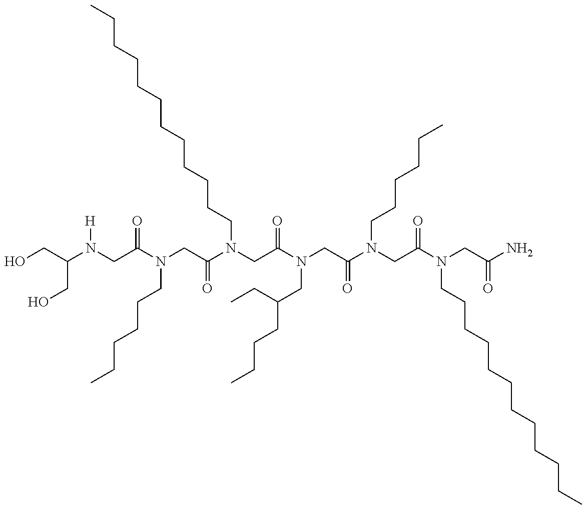 [SMILES: CCCCCCCCCCCCN(CC(N)=O)C(=O)CN(CCCCCC)C(=O)CN(CC(CC)CCCC)C(=O)CN(CCCCCCCCCCCC)C(=O)CN(CCCCCC)C(=O)CNC(CO)CO] |
| 46 | 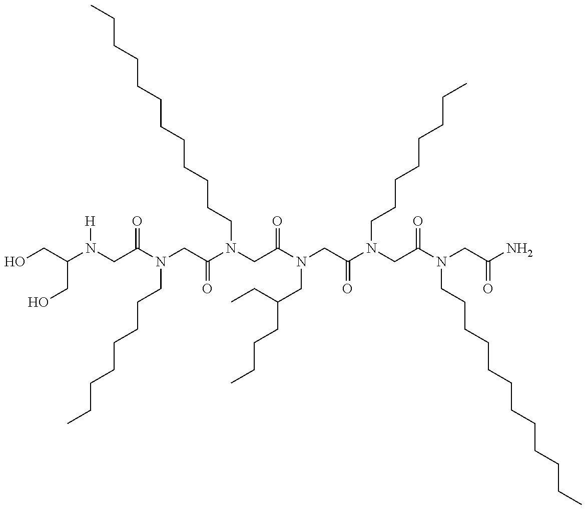 [SMILES: CCCCCCCCCCCCN(CC(N)=O)C(=O)CN(CCCCCCCC)C(=O)CN(CC(CC)CCCC)C(=O)CN(CCCCCCCCCCCC)C(=O)CN(CCCCCCCC)C(=O)CNC(CO)CO] |
| 47 | 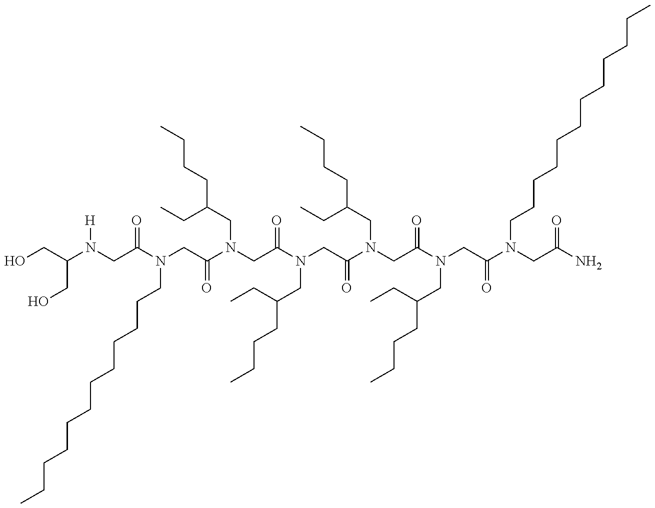 [SMILES: CCCCCCCCCCCCN(CC(N)=O)C(=O)CN(CC(CC)CCCC)C(=O)CN(CC(CC)CCCC)C(=O)CN(CC(CC)CCCC)C(=O)CN(CC(CC)CCCC)C(=O)CN(CCCCCCCCCCCC)C(=O)CNC(CO)CO] |
| 48 | 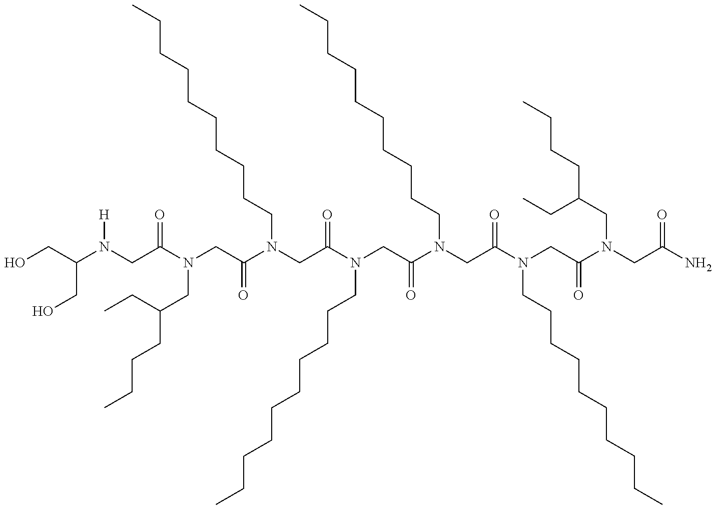 [SMILES: CCCCCCCCCCN(CC(=O)N(CCCCCCCCCC)CC(=O)N(CC(N)=O)CC(CC)CCCC)C(=O)CN(CCCCCCCCCC)C(=O)CN(CCCCCCCCCC)C(=O)CN(CC(CC)CCCC)C(=O)CNC(CO)CO] |
| 49 | 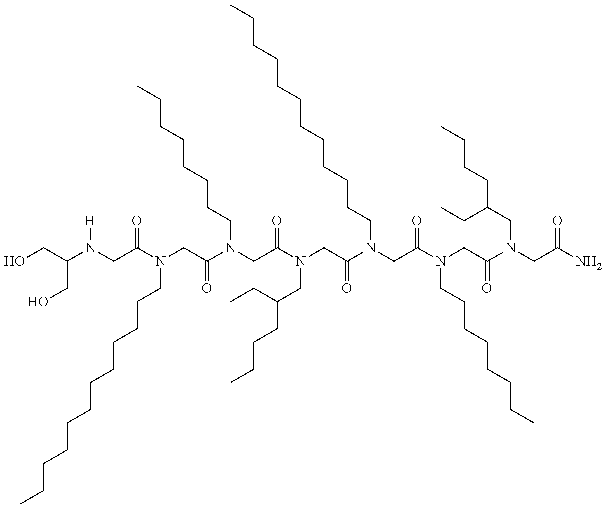 [SMILES: CCCCCCCCCCCCN(CC(=O)N(CCCCCCCC)CC(=O)N(CC(=O)N(CCCCCCCCCCCC)CC(=O)N(CCCCCCCC)CC(=O)N(CC(N)=O)CC(CC)CCCC)CC(CC)CCCC)C(=O)CNC(CO)CO] |
| 51 | 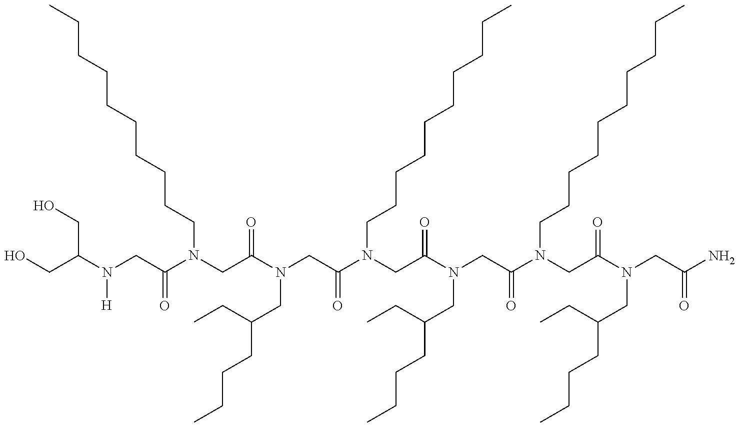 [SMILES: CCCCCCCCCCN(CC(=O)N(CC(=O)N(CCCCCCCCCC)CC(=O)N(CC(=O)N(CCCCCCCCCC)CC(=O)N(CC(N)=O)CC(CC)CCCC)CC(CC)CCCC)CC(CC)CCCC)C(=O)CNC(CO)CO] |
| 52 | 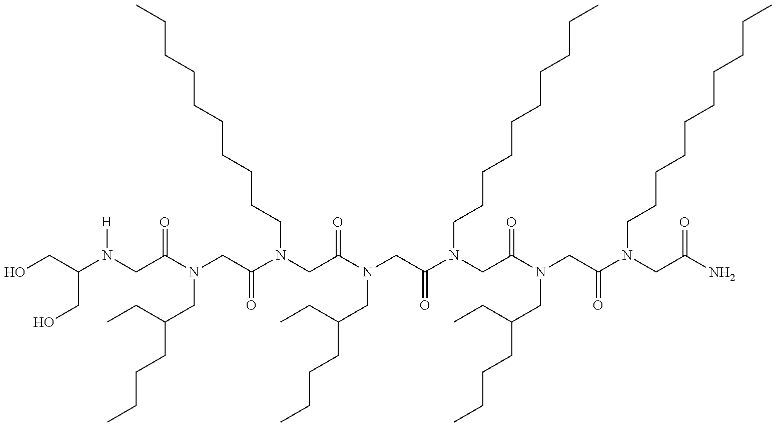 [SMILES: CCCCCCCCCCN(CC(N)=O)C(=O)CN(CC(CC)CCCC)C(=O)CN(CCCCCCCCCC)C(=O)CN(CC(CC)CCCC)C(=O)CN(CCCCCCCCCC)C(=O)CN(CC(CC)CCCC)C(=O)CNC(CO)CO] |
| 53 | 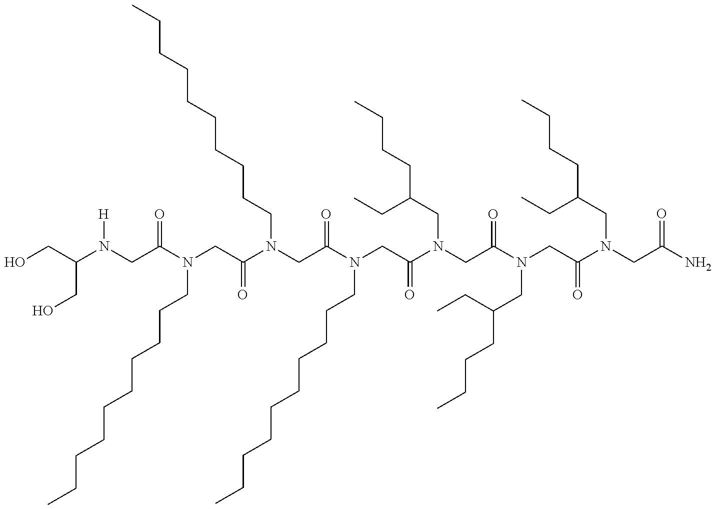 [SMILES: CCCCCCCCCCN(CC(=O)N(CCCCCCCCCC)CC(=O)N(CCCCCCCCCC)CC(=O)N(CC(=O)N(CC(=O)N(CC(N)=O)CC(CC)CCCC)CC(CC)CCCC)CC(CC)CCCC)C(=O)CNC(CO)CO] |
| 54 | 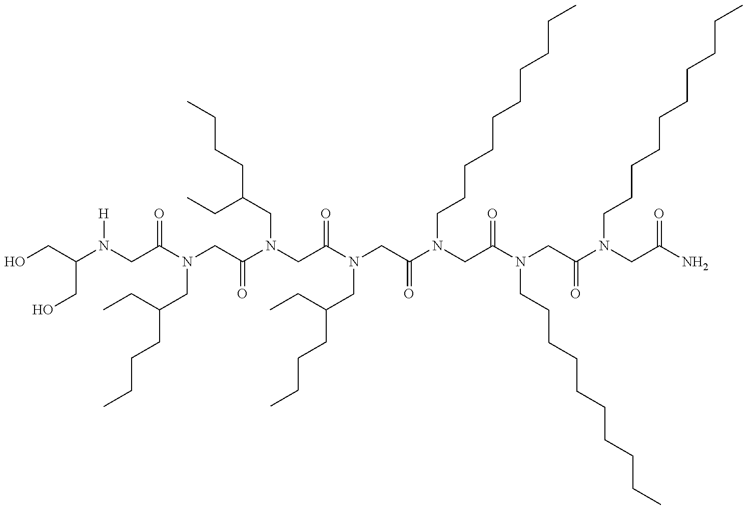 [SMILES: CCCCCCCCCCN(CC(N)=O)C(=O)CN(CCCCCCCCCC)C(=O)CN(CCCCCCCCCC)C(=O)CN(CC(CC)CCCC)C(=O)CN(CC(CC)CCCC)C(=O)CN(CC(CC)CCCC)C(=O)CNC(CO)CO] |
| 56 | 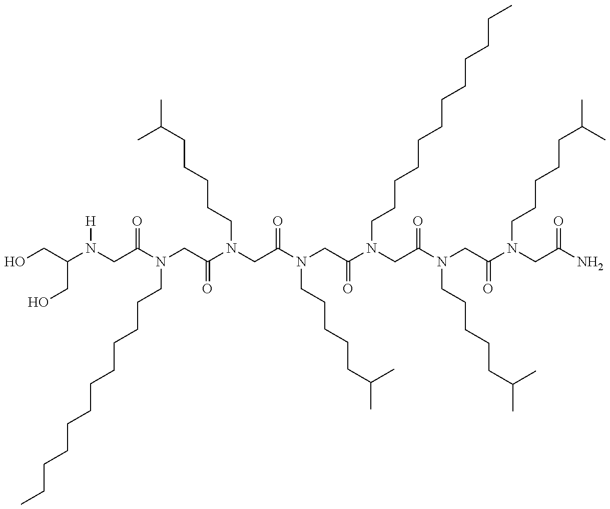 [SMILES: CCCCCCCCCCCCN(CC(=O)N(CCCCCC(C)C)CC(=O)N(CCCCCC(C)C)CC(=O)N(CCCCCCCCCCCC)CC(=O)N(CCCCCC(C)C)CC(=O)N(CCCCCC(C)C)CC(N)=O)C(=O)CNC(CO)CO] |
| 57 | 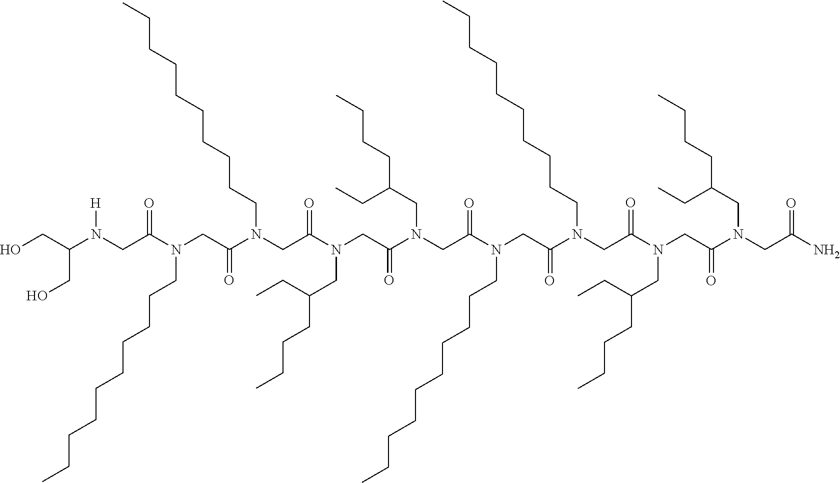 [SMILES: CCCCCCCCCCN(CC(=O)N(CCCCCCCCCC)CC(=O)N(CC(=O)N(CC(=O)N(CCCCCCCCCC)CC(=O)N(CCCCCCCCCC)CC(=O)N(CC(=O)N(CC(N)=O)CC(CC)CCCC)CC(CC)CCCC)CC(CC)CCCC)CC(CC)CCCC)C(=O)CNC(CO)CO] |
| 58 | 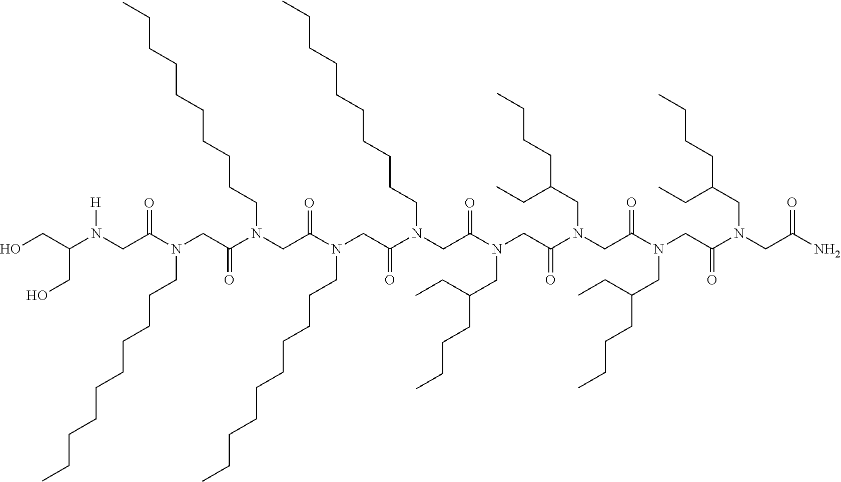 [SMILES: CCCCCCCCCCN(CC(=O)N(CCCCCCCCCC)CC(=O)N(CCCCCCCCCC)CC(=O)N(CCCCCCCCCC)CC(=O)N(CC(=O)N(CC(=O)N(CC(=O)N(CC(N)=O)CC(CC)CCCC)CC(CC)CCCC)CC(CC)CCCC)CC(CC)CCCC)C(=O)CNC(CO)CO] |
| 59 | 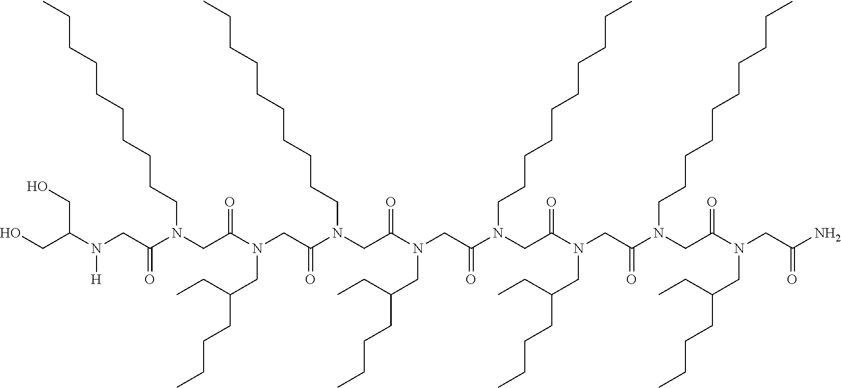 [SMILES: CCCCCCCCCCN(CC(=O)N(CC(=O)N(CCCCCCCCCC)CC(=O)N(CC(=O)N(CCCCCCCCCC)CC(=O)N(CC(=O)N(CCCCCCCCCC)CC(=O)N(CC(N)=O)CC(CC)CCCC)CC(CC)CCCC)CC(CC)CCCC)CC(CC)CCCC)C(=O)CNC(CO)CO] |
| 60 | 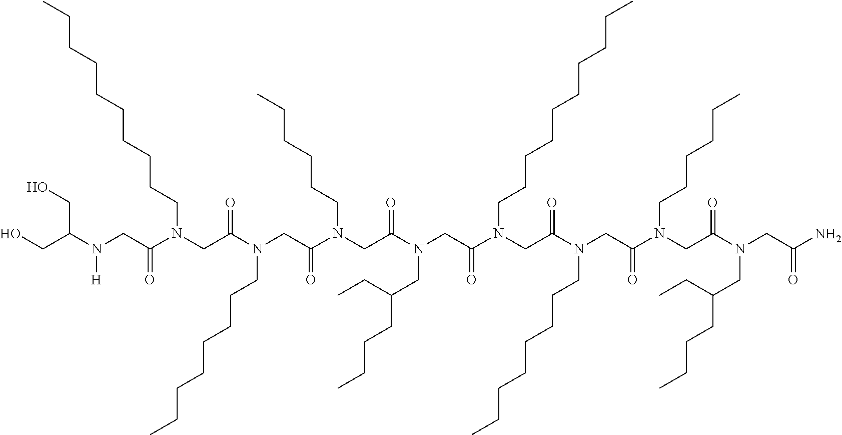 [SMILES: CCCCCCCCCCN(CC(=O)N(CCCCCCCC)CC(=O)N(CCCCCC)CC(=O)N(CC(=O)N(CCCCCCCCCC)CC(=O)N(CCCCCCCC)CC(=O)N(CCCCCC)CC(=O)N(CC(N)=O)CC(CC)CCCC)CC(CC)CCCC)C(=O)CNC(CO)CO] |
| 61 | 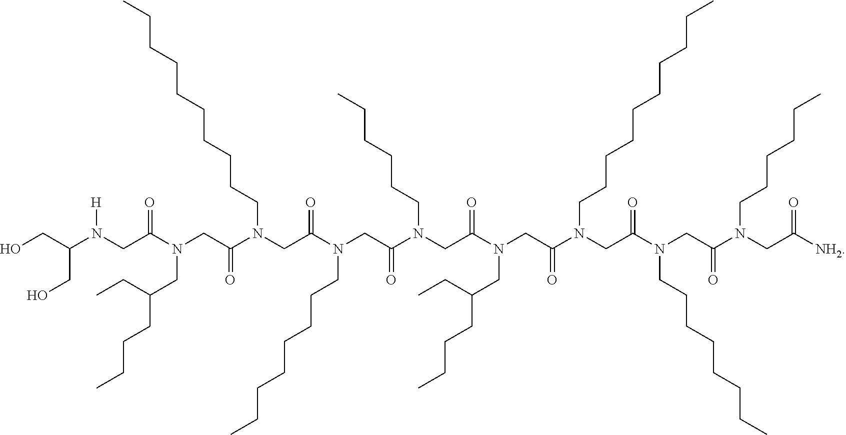 [SMILES: CCCCCCCCCCN(CC(=O)N(CCCCCCCC)CC(=O)N(CCCCCC)CC(=O)N(CC(=O)N(CCCCCCCCCC)CC(=O)N(CCCCCCCC)CC(=O)N(CCCCCC)CC(N)=O)CC(CC)CCCC)C(=O)CN(CC(CC)CCCC)C(=O)CNC(CO)CO] |

18. The compound of claim 17, having the structure of Compound 1, 6, 12, 21, 30, 34, 35, or 41.

19.-25. (canceled)

26. A pharmaceutically acceptable salt of the compound of claim 1.

27. A delivery vehicle composition comprising the compound of claim 1.

28.-83. (canceled)

84. A delivery vehicle complex comprising the delivery vehicle composition of claim 27, and a polyanionic compound.

85-162. (canceled)

163. A pharmaceutical composition comprising the delivery vehicle complex of claim 84, and a pharmaceutically acceptable excipient.

164. (canceled)

165. A method of inducing an immune response in a subject in need thereof, comprising administering to the subject an effective amount of the delivery vehicle complex of claim 84, thereby inducing an immune response in the subject.

166. A method of treating a viral infection in a subject in need thereof, comprising administering to the subject an effective amount of the delivery vehicle complex of claim 84, thereby treating the viral infection in the subject.

167.-169. (canceled)

170. A method of delivering a polyanionic compound to a cell comprising contacting the cell with the delivery vehicle complex of claim 84.

171.-172. (canceled)

173. A method of forming the delivery vehicle complex of claim 84, comprising contacting the compound or salt of Formula (I) with the polyanionic compound.

174. (canceled)

175. A vaccine comprising the delivery vehicle complex of claim 84.

176. (canceled)

177. A method of treating or preventing cancer in a patient, comprising administering to the patient the vaccine of claim 175.

178. (canceled)
